(19) United States
(12) Patent Application Publication
(10) Pub. No.: US 2023/0357320 A1
(43) Pub. Date: Nov. 9, 2023

(54) HELICAL STAPLED PEPTIDES AND USES THEREOF

(71) Applicant: FOG PHARMACEUTICALS, INC., Cambridge, MA (US)

(72) Inventors: Gregory L. Verdine, Boston, MA (US); John Hanney McGee, Somerville, MA (US); Santosh Kumar Choudary, Medford, MA (US); Yue-Mei Zhang, Wellesley, MA (US); Martin Robert Tremblay, Melrose, MA (US)

(21) Appl. No.: 17/912,029

(22) PCT Filed: Mar. 17, 2021
(86) PCT No.: PCT/US21/22744

§ 371 Date: Sep. 15, 2022

Related U.S. Application Data
(60) Provisional application No. 62/991,032, filed on Mar. 17, 2020.

(51) Int. Cl. C07K 7/06

(52) U.S. Cl. CPC C07K 7/06; A61K 38/00

(57) ABSTRACT

In some embodiments, the present disclosure provides stapled peptides and compositions thereof. In some embodiments, provided peptides can bind to and modulate functions of estrogen receptor. In some embodiments, the present disclosure provides technologies for treating various conditions, disorders or diseases

### CROSS-REFERENCE TO RELATED APPLICATIONS

This application claims priority to U.S. Provisional Application No. 62/991,032, filed Mar. 17, 2020, the entirety of which is incorporated herein by reference.

### BACKGROUND

The present disclosure relates to the fields of biology and chemistry, particularly cellular biology and organic chemistry.

Recent advances in identifying human disease targets have not been matched by accompanying advances in the ability to drug them. This is largely a consequence of the shortcomings of the two main classes of approved therapeutics, biologics and small molecules (see, e.g., Verdine and Walensky, Clinical Cancer Research 2007, 13 (24), 7264). Biologics, despite an impressive ability to engage diverse target proteins, are largely restricted to an extracellular operating theatre, as their size and polarity renders them unable to cross biological membranes (Carter and Lazar, Nature Reviews Drug Discovery 2018, 17 (3), 197-223; Nature Reviews Cancer 2012, 12 (4), 278-287). Small molecules, in contrast, are capable of accessing the intracellular space, but cannot bind with high affinity to the vast majority of proteins that are found there (Jin et al., Annual Review of Pharmacology and Toxicology 2014, 54 (1), 435-456; Ran and Gestwicki, Current Opinion in Chemical Biology 2018, 44, 75-86.

Thus, there is a need to connect the ability to identify disease targets with the ability to drug them with a new class of drugs that can cross the cell membrane and bind with high affinity to intracellular targets.

### SUMMARY

Among other things, the present disclosure provides molecules that are able to cross biological membranes and bind intracellular targets with high affinity. In some embodiments, such molecules are or comprise peptides. In some embodiments, such molecules are or comprise stapled peptides.

In some embodiments, the present disclosure provides various compounds which are peptides. In some embodiments, provided peptides are useful for preparing stapled peptides, e.g., through metathesis. In some embodiments, the present disclosure provides stapled peptides that demonstrate various advantages, e.g., adjusted membrane permeability, reduced number of N-terminus unmasked amide NH, etc.

In some embodiments, a provided peptide comprises an amino acid residue B¹ as described herein. In some embodiments, a peptide comprises a residue having the structure of P-I or a salt form thereof. In some embodiments, a peptide comprises a residue having the structure of P-II or a salt form thereof. In some embodiments, a peptide comprises a residue having the structure of P-III or a salt form thereof.

In some embodiments, a provided peptide is or comprises B-X²-Z-J-X⁵-X⁶-Z-X⁸-X⁹-X¹⁰-X¹¹-X¹²-X¹³, or a salt thereof, wherein each variable is independently as described herein.

In some embodiments, a provided peptide is or comprises B′-X²-Z-J′-X⁵-X⁶-Z-X⁸-X⁹-X¹⁰-X¹¹-X¹²-X¹³, or a salt thereof, wherein each variable is independently as described herein.

In some embodiments, a provided peptide is or comprises B-Z-X³-J-X⁵-Z-X⁷-X⁸-X⁹-X¹⁰-X¹¹-X¹²-X¹³, or a salt thereof, wherein each variable is independently as described herein.

In some embodiments, a provided peptide is or comprises B′-Z-X³-J′-X⁵-Z-X⁷-X⁸-X⁹-X¹⁰-X¹¹-X¹²-X¹³, or a salt thereof, wherein each variable is independently as described herein.

In some embodiments, a provided peptide is or comprises B-X²-X³-J-X⁵-X⁶-X⁷-O-X⁹-X¹⁰-X¹¹-X¹²-X¹³, or a salt thereof, wherein each variable is independently as described herein.

In some embodiments, a provided peptide is or comprises B′-X²-X³-J″-X⁵-X⁶-X⁷-O′-X⁹-X¹⁰-X¹¹-X¹²-X¹³, or a salt thereof, wherein each variable is independently as described herein.

In some embodiments, a provided peptide is or comprises B-X²-X³-J-X⁵-X⁶-X⁷-X⁸-X⁹-X¹⁰-O-X¹²-X¹³-X¹⁴, or a salt thereof, wherein each variable is independently as described herein.

In some embodiments, a provided peptide is or comprises B′-X²-X³-J″-X⁵-X⁶-X⁷-X⁸-X⁹-X¹⁰-O′-X¹²-X¹³-X¹⁴, or a salt thereof, wherein each variable is independently as described herein.

In some embodiments, a provided peptide has the structure of:

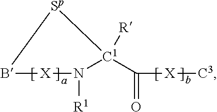

or a salt thereof, wherein each variable is independently as described herein.

In some embodiments, a provided peptide has the structure of.

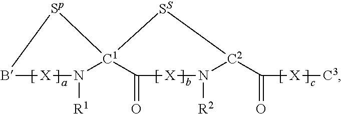

or a salt thereof, wherein each variable is independently as described herein.

In some embodiments, the present disclosure provides a pharmaceutical composition which comprises or delivers a peptide, or a pharmaceutically acceptable salt thereof, and a pharmaceutically acceptable carrier and/or a pharmaceutically acceptable excipient.

In some embodiments, the present disclosure provides methods for modulating one or more functions and/or properties of an estrogen receptor, comprising administering to a system comprising the estrogen receptor a provided peptide or composition. In some embodiments, the present disclosure provides methods for modulating one or more functions and/or properties of an estrogen receptor, comprising contacting the estrogen receptor a provided peptide or composition.

Provided technologies, among other things, are useful for preventing or treating various conditions, disorders or diseases. In some embodiments, the present disclosure provides methods for treating conditions, disorders or diseases associated with estrogen receptor. In some embodiments, the present disclosure provides methods for treating or preventing a condition, disorder or disease, comprising administering to a subject suffering therefrom or susceptible thereto an effective amount of a provided peptide or composition. In some embodiments, the present disclosure provides methods for treating a condition, disorder or disease, comprising administering to a subject suffering therefrom a therapeutically effective amount of a provided peptide or composition. In some embodiments, a condition, disorder or disease is cancer.

### BRIEF DESCRIPTION OF THE DRAWINGS

FIGS. 1A and 1B are schematic representations of an α-helical peptide. In FIG. 1A, the schematic shows the N-acetyl capped helical peptides having four unsatisfied N-terminal amide NH bonds (shown as grey spheres) while the rest of the amide NH bonds are optionally and independently part of the internal hydrogen bonding network intrinsic to helical peptides. FIG. 1B provides an expanded view of the five N-terminal amide NH bonds in helical peptides of FIG. 1A.

FIGS. 1C and 1D are schematic of non-limiting stapled peptides of the present disclosure. FIG. 1C shows how ProLock stapled peptides are designed to possess only one unsatisfied amide N-terminal NH bond. FIG. 1D shows an expanded view of the five N-terminal amide NH bonds in ProLock stapled peptides in FIG. 1C. Only the amide NH of the i+1 residue remains easily accessible to solvent waters

FIG. 2 is a schematic drawing showing a synthesis scheme of N-acetyl-PL3-OH (“PL3”). L-proline formed oxazolidinone-1 with chloral hydrate under reflux conditions.

FIGS. 3A and 3B are an X-ray crystal structure of the Estrogen Receptor Binding Domain (ER LBD) (FIG. 3A) and a table showing the sequences of the ligands that bind to the ERLBD (FIG. 3B).

FIG. 4 is a schematic drawing showing the PAMP assay plate system. The PAMPA plate system is composed of two compartments, donor and acceptor, which are separated by a phospholipid-infused membrane.

FIG. 5 is a line graph showing the direct fluorescence polarization (FP) values of an FITC-ERL4 probe with ERβ LBD. The K_{D} of the probe was determined to be 160 nM with a 1:1 binding model with Hill slope. The FP values are mean±S.E.M of two independent replicates.

FIG. 6 is a line graph showing the Competition FP data of first generation ProLock stapled peptides. The high and low polarization data from each assay plate was used to normalize the data, which were fit to a 1:1 binding model with Hill slope to determine EC₅₀. The normalized FP values are mean±S.E.M of at least four independent replicates.

FIG. 7 is a line graph showing a calibration curve of ten standard compounds that was used to determine CHI Log D. The retention time values are mean±S.E.M of two independent replicates.

FIG. 8 is a schematic showing the structures of some non-limiting amino acid residue analogs as described herein.

FIG. 9A is a schematic showing the stapling reaction for various non-limiting stapled peptides as described herein.

FIG. 9B are a series of line graphs showing the percent product conversion of six peptides at two ring closing metathesis (RCM) temperatures (room temperature and 37° C.) in two catalyst concentrations (15 mol % Grubbs-I and 25 mol % Grubbs-I) at four different timepoints (30 min, 60 min, 120 min, and 240 min).

FIG. 9C is a bar graph showing the percent product conversion and isomer distribution of three peptides with S3, S4 and S5 stapling amino acids at position 4, at two RCM temperatures, and two catalyst concentrations, all after 240 minutes. The percent product conversion bar graphs are split based on the isomer distribution with each staple type.

FIG. 9D is a line graph showing the CD spectra of all five ProLock stapled products from FIG. 9C. Based on the CD analysis, PL3-S5 stapled ProLock peptide isomer 2 has a canonical α-helical structure.

FIG. 9E is a line graph showing the CD spectra of first-generation ProLock stapled peptides. All of the first-generation peptides except PLL4-4 adopt a helical conformation, as determined by the presence of two minima in the spectra at 208 and 222 nm.

FIG. 10A is a line graph showing the results of NMR solution analysis of the PLL4-5 ProLock stapled peptide, a non-limiting stapled peptide of the present disclosure. The peaks corresponding to two olefin protons of PLL4-5 are used to determine the geometry of the alkenyl bond. The J-coupling values of 10.1 and 11.7 Hz implicated that the olefin of isomer-2 stapled product of PLL4-5 peptide adopts a cis-conformation.

FIG. 10B is a schematic diagram showing an ensemble of five lowest energy structures of PLL4-5. The overlaid structures indicate that the peptide adopts an α-helical fold through the first 1.5 turns. Backbone RMSD of this overlay was 0.49 Å (RMSD was calculated using Protein Consensus tool in MOE software).

FIG. 11 is a schematic diagram showing five design variants of six ProLock stapled peptides were tested in the PAMPA assay to assess the importance of the ProLock design components. The design features examined were the N-terminal acetyl cap (capped vs. no-cap peptide), the remaining solvent-exposed amide NH bond of the i+1 residue (normal vs. depsi-linked peptide), the C-terminal methyl amide cap (methyl amide vs. amide cap), and a ProLock staple (ProLock stapled vs. unstapled vs S5-S5 stapled peptide).

### DETAILED DESCRIPTION OF CERTAIN EMBODIMENTS

The aberrant expression and/or function of certain intracellular molecules is known to be associated with diseases such as cancer (Sever and Brugge, Cold Spring Harb Perspect Med 2015; 5:a006098). For example, members of the myc oncogene family are dysregulated in over 50% of human cancers (Chen et al., Signal Transduction and Targeted Therapy (2018) 3: 5); however, to date, there is no FDA approved therapeutic targeting myc for treating human cancer. Other intracellular targets are known to drive human diseases, and yet are unable to be drugged-the so-called “undruggable” targets. At least part of reason that these intracellular targets are undruggable is because they lack a deep hydrophobic pocket normally required for small molecule binding. In other words, small molecule therapeutics, while able to cross the cell membrane and thus access the intracellular target, are unable to bind to the target with high enough affinity to effectively modulate that target and thus modulate the growth and/or behavior of the cell bearing that target.

In nature, such “undruggable” targets (e.g., proteins) are often bound by macrocyclic molecules, frequently peptidic in structure, whose large size compared to small molecules enables them to bind with high affinity and specificity to the surfaces of such targets (Dang and Süssmuth, Accounts of Chemical Research 2017, 50 (7), 1566-1576; Naylor et al., Current Opinion in Chemical Biology 2017, 38, 141-147). Significant efforts have been made to elucidate the mechanisms of cell entry for these natural products, which often possess molecular weights of 700-1200 Da or higher, well beyond the typical range for cell penetration in small molecule drug discovery (Bockus et al., Journal of Medicinal Chemistry 2015, 58 (18), 7409-7418; Andrew et al., Current Topics in Medicinal Chemistry 2013, 13 (7), 821-836; Whitty et al., Drug Discovery Today 2016, 21 (5), 712-717; Over et al., Nature Chemical Biology 2016, 12 (12), 1065-1074; Wang and Craik, Peptide Science 2016, 106 (6), 901-909; Matsson and Kihlberg, Journal of Medicinal Chemistry 2017, 60 (5), 1662-1664).

While the mechanisms of cell entry are complex and vary from molecule to molecule, a substantial body of research on peptidic macrocycles has highlighted one key trend: the importance of amide proton cloaking in permitting passive membrane permeability (Rader et al, Bioorganic & Medicinal Chemistry 2018, 26 (10), 2766-2773; Bockus et al., Journal of Medicinal Chemistry 2015, 58 (11), 4581-4589; Hickey et al., Journal of Medicinal Chemistry 2016, 59 (11), 5368-5376). The amide proton, present between every residue in a polypeptide chain, is highly electropositive and forms a strong interaction with water. This strong interaction poses a substantial hurdle for passive membrane permeability, since waters must be shed prior to entering the lipid bilayer. Exposed amide groups incur a further energetic penalty upon membrane entry due to their unfavorable electrostatic interaction low-dielectric environment of the membrane interior (Ahlbach et al., Future Med Chem 2015, 7 (16), 2121-2130). Consequently, most peptides and proteins are unable to passively cross membranes (Yang and Hinner, Methods Mol Biol 2015, 1266, 29-53).

In peptide macrocycles that exhibit passive membrane permeability, these problematic amide protons are typically removed either by replacement of the amide-NH with O (so-called depsipeptide linkage), replacement of the amide proton itself with a methyl group, or cloaking of the amide proton from solvent water through the formation of intramolecular hydrogen bonds between the amide proton groups and a hydrogen bond-accepting group elsewhere in the molecule (often a carbonyl) (Rader and Reichart, Bioorganic & Medicinal Chemistry 2018, 26 (10), 2766-2773: Ahlbach et al., Future Med Chem 2015, 7 (16), 2121-2130; Wang et al. The Journal of Physical Chemistry B 2018, 122 (8), 2261-2276; Rezai et al., Journal of the American Chemical Society 2006, 128 (43), 14073-14080; Rezai et al., Journal of the American Chemical Society 2006, 128 (8), 2510-2511; Biron et al., Angewandte Chemie International Edition 2008, 47 (14), 2595-2599). Indeed, the paradigmatic example of a peptide macrocycle that exhibits robust cytoplasmic exposure, cyclosporine A (CsA), employs both N-methylation and cloaking through transannular hydrogen bonding (Ahlbach et al., Future Med Chem 2015, 7 (16), 2121-2130). Extensive work by several research groups has shown that these strategies can be applied as design principles to endow artificial macrocycles with the ability to passively cross membranes (Rader et al, Bioorganic & Medicinal Chemistry 2018, 26 (10), 2766-2773; Hickey et al., Journal of Medicinal Chemistry 2016, 59 (11), 5368-5376; Biron et al., Angewandte Chemie International Edition 2008, 47 (14), 2595-2599; Hewitt et al., Journal of the American Chemical Society 2015, 137 (2), 715-721; Beck et al., Journal of the American Chemical Society 2012, 134 (29), 12125-12133; Thansandote et al., Bioorganic & Medicinal Chemistry 2015, 23 (2), 322-327).

In the context of folded proteins, nature has offered an alternative structural solution to the problem of amide proton cloaking: the α-helix, a protein secondary structure that is defined by repeating intramolecular hydrogen bonds between the amide proton group of one residue and the carbonyl of the amino acid located 3 residues N-terminal to it. The intrinsic ability of α-helices to cloak their own amide protons explains their widespread prevalence in natural transmembrane (TM) proteins (White and Wimley, Annu. Rev. Biophys. Biomolec. Struct. 1999, 28, 319-365: Heyden et al., Soft Matter 2012, 8 (30), 7742-7752). Nuclear-encoded TM proteins in eukaryotes are almost exclusively α-helical, and the only alternative TM fold found in nature is the bacterially-derived beta-barrel, which also cloaks amide protons with an intramolecular hydrogen bonding network, albeit in an significantly larger structure than single α-helices that is impractical for the development of synthetic drugs (Vinothkumar et al., Q Rev Biophys 2010, 43 (1), 65-158).

Just as CsA has served as the inspiration for design of mimetic heat-to-tail cyclized peptide ligands, so have proteinaceous α-helices inspired efforts to recapitulate nature's design features in small, synthetic peptides having an α-helical conformation hyperstabilized through the incorporation of a structural brace, also known as a “staple” (Walensky and Bird, Journal of Medicinal Chemistry 2014, 57 (15), 6275-6288; Verdine and Hilinski, “Stapled peptides for intracellular drug targets”. In Methods in Enzymology: Protein Engineering for Therapeutics, Vol 203, Pt B, Wittrup, K. D.; Verdine, G. L., Eds. Elsevier Academic Press Inc: San Diego, 2012; Vol. 503, pp 3-33; Schafmeister et al., Journal of the American Chemical Society 2000, 122 (24), 5891-5892). One of these, the all-hydrocarbon staple, first discovered in these laboratories, has been extensively studied and is the basis for a drug candidate that targets the challenging nucleocytoplasmic proteins HDM2 and HDMX, is currently undergoing Phase II clinical trails (Chang et al., Proceedings of the National Academy of Sciences 2013, 110 (36), E3445-E3454).

However, obtaining robust, passive cytoplasmic exposure in existing α-helical stapling systems remains a formidable challenge (Sawyer et al., Bioorganic & Medicinal Chemistry 2018, 26 (10), 2807-2815. Among other things, the present disclosure encompasses the recognition that while these prior systems may do an effective job of cloaking the amide protons within the body of the α-helix, they fail to address exposure of amide protons at the N-terminal end of the α-helix. The present disclosure stems from the insight that reducing the number of “uncloaked” N-terminal amide protons, while also maintaining conformational stabilization of the α-helix, facilitate passive membrane permeation. In some embodiments, the present disclosure provides stapled peptides with reduced number of uncloaked N-terminal amide protons. In some embodiments, the present disclosure provides stapled peptides with a reduced number of “free” N-terminal amide protons which form hydrogen-bonds with water when exposed to water. In some embodiments, a reduction is to no more than one. As demonstrated herein, in many embodiments, provided stapled peptides can possess helical structures and provide various advantages.

In some embodiments, a provided technology (e.g., a peptide, composition, method, etc.) stems from the development of a novel stapling system, ProLock™, that stabilizes peptides in an α-helical conformation while also reducing the number of solvent-exposed amide protons at the peptide N-terminus. Incorporation of a ProLock™ staple into biologically relevant sequences can endow them with the ability to passively cross membranes at levels comparable to some orally bioavailable drugs, while retaining the ability to bind their protein target with low- or sub-micromolar affinity. Surprisingly, as demonstrated herein, even ProLock™ stapled peptides with multiple polar and charged sidechains were found to exhibit robust levels of passive membrane permeability. These results allow the targeting of proteins that require polar or charged functionality for effective ligand binding.

In some embodiments, the present disclosure is directed to an α-helix stapling system, a ProLock™ stapling system (trademarked by Fog Pharmaceuticals, Inc.) which is designed to enable the passive permeability of α-helical peptides by removing or cloaking one or more (e.g., in some embodiments, three of their four) unsatisfied N-terminal amide protons, and by nucleating and stabilizing helix formation. In some embodiments, the present disclosure provides peptides comprising such a stapling system, and compositions and methods thereof.

In some embodiments, the present disclosure is directed to a stapling system comprising two staples or more staples in the same peptide. In some embodiments, a single amino acid is attached to two staples. In some embodiments, at least one of the two or more staples in the same peptide is a ProLock™ staple.

In various embodiments, the present disclosure provides compounds, e.g., stapled peptides, that are able to cross biological membranes (e.g., cell membranes) and also bind with high affinity to intracellular targets.

The published patents, patent applications, websites, company names, and scientific literature referred to herein establish the knowledge that is available to those with skill in the art and are hereby incorporated by reference in their entirety to the same extent as if each was specifically and individually indicated to be incorporated by reference. Any conflict between any reference cited herein and the specific teachings of this specification shall be resolved in favor of the latter.

Terms defined or used in the description and the claims shall have the meanings indicated, unless context otherwise requires. Technical and scientific terms used herein have the meaning commonly understood by one of skill in the art to which the present disclosure pertains, unless otherwise defined. Any conflict between an art-understood definition of a word or phrase and a definition of the word or phrase as specifically taught in this specification shall be resolved in favor of the latter. As used herein, the following terms have the meanings indicated. As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds, amino acids, and polypeptides described herein can comprise one or more asymmetric centers, and thus can exist in various isomeric forms, e.g., enantiomers and/or diastereomers. For example, the compounds, amino acids, and polypeptides described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H. Tables of Resolving Agents and Optical Resolutions p. 268 (E. L. Eliel, Ed., Univ. of Notre Dame Press, Notre Dame, IN 1972). The present disclosure additionally encompasses compounds, amino acids, and polypeptides described herein as individual isomers substantially free of other isomers, and alternatively, as mixtures of various isomers.

As used throughout the present disclosure, when a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl. Likewise, the phrase “aliphatic groups containing 1-6 aliphatic carbon atoms” is intended to encompass aliphatic groups contain 1 aliphatic carbon atom, 2 aliphatic carbon atoms, 3 aliphatic carbon atoms, 4 aliphatic carbon atoms, 5 aliphatic carbon atoms, or 6 aliphatic carbon atoms.

The term “aliphatic,” as used herein, means a straight-chain (i.e., unbranched) or branched, substituted or unsubstituted hydrocarbon chain that is completely saturated or that contains one or more units of unsaturation, or a substituted or unsubstituted monocyclic, bicyclic, or polycyclic hydrocarbon ring that is completely saturated or that contains one or more units of unsaturation, or combinations thereof. Unless otherwise specified, aliphatic groups contain 1-100 aliphatic carbon atoms. In some embodiments, aliphatic groups contain 1-30 aliphatic carbon atoms. In some embodiments, aliphatic groups contain 1-20 aliphatic carbon atoms. In other embodiments, aliphatic groups contain 1-10 aliphatic carbon atoms. In other embodiments, aliphatic groups contain 1-9 aliphatic carbon atoms. In other embodiments, aliphatic groups contain 1-8 aliphatic carbon atoms. In other embodiments, aliphatic groups contain 1-7 aliphatic carbon atoms. In other embodiments, aliphatic groups contain 1-6 aliphatic carbon atoms. In still other embodiments, aliphatic groups contain 1-5 aliphatic carbon atoms, and in yet other embodiments, aliphatic groups contain 1, 2, 3, or 4 aliphatic carbon atoms. Suitable aliphatic groups include, but are not limited to, linear or branched, substituted or unsubstituted alkyl, alkenyl, alkynyl groups and hybrids thereof, as well as cycloalkyl and cycloalkenyl groups. In some embodiments, an aliphatic group is optionally substituted with one or more functional groups. As will be appreciated by one of ordinary skill in the art, “aliphatic” is intended herein to include alkyl, alkenyl, alkynyl, cycloalkyl, and cycloalkenyl moieties.

The term “heteroaliphatic”, as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; heteroarylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓₐ; —OCO₂Rₓₐ; —OCON(Rₓₐ)₂; —N(Rₓₐ)₂; —S(O)₂Rₓₐ; —NRₓₐ(CO)Rₓₐ, wherein each occurrence of Rₓₐ independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substitutents are illustrated by the specific embodiments shown in the Examples that are described herein.

As used herein, “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls). In the absence of any numerical designation, “alkyl” is a chain (straight or branched) having 1 to 20 (inclusive) carbon atoms in it.

In some embodiments, alkyl has 1-100 carbon atoms. In certain embodiments, a straight chain or branched chain alkyl has about 1-20 carbon atoms in its backbone (e.g., C₁-C₂₀ for straight chain, C₂-C₂₀ for branched chain). In some embodiments, an alkyl group has 1 to 20 carbon atoms (“C₁₋₂₀alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted C₁₋₁₀ alkyl (e.g., —CH₃). In certain embodiments, the alkyl group is a substituted C₁₋₁₀ alkyl.

“Perhaloalkyl” is a substituted alkyl group as defined herein wherein all of the hydrogen atoms are independently replaced by a halogen, e.g., fluoro, bromo, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁₋₄ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₂ perhaloalkyl”). In some embodiments, all of the hydrogen atoms are replaced with fluoro. In some embodiments, all of the hydrogen atoms are replaced with chloro. Examples of perhaloalkyl groups include —CF₃, —CF₂CF₃, —CF₂CF₂CF₃, —CCl₃, —CFCl₂, —CF₂Cl, and the like.

As used herein, “heteroalkyl” refers to a radical of a straight-chain or branched saturated hydrocarbon group having from 1 to 30 carbon atoms, and which further comprises 1-10 heteroatoms independently selected from oxygen, nitrogen, and sulfur included within the parent chain (“C₁₋₃₀ heteroalkyl”). In some embodiments, a heteroalkyl group has 1 to 20 carbon atoms and 1-10 heteroatoms, inclusive (“C₁₋₂₀ heteroalkyl”). In some embodiments, a heteroalkyl group has 1 to 20 carbon atoms and 1-10 heteroatoms, inclusive (“C₁₋₁₀ heteroalkyl”). In some embodiments, a heteroalkyl group has 1 to 9 carbon atoms and 1-6 heteroatoms, inclusive (“C₁₋₉ heteroalkyl”). In some embodiments, a heteroalkyl group has 1 to 8 carbon atoms and 1-5 heteroatoms, inclusive (“C₁₋₈ heteroalkyl”). In some embodiments, a heteroalkyl group has 1 to 7 carbon atoms, and 1-4 heteroatoms, inclusive (“C₁₋₇ heteroalkyl”). In some embodiments, a heteroalkyl group has 1 to 6 carbon atoms and 1-3 heteroatoms, inclusive (“C₁₋₆ heteroalkyl”). In some embodiments, a heteroalkyl group has 1 to 5 carbon atoms and 1-2 heteroatoms, inclusive (“C₁₋₅ heteroalkyl”). In some embodiments, a heteroalkyl group has 1 to 4 carbon atoms and 1-2 heteroatoms, inclusive (“C₁₋₄ heteroalkyl”). In some embodiments, a heteroalkyl group has 1 to 3 carbon atoms and 1-2 heteroatoms, inclusive (“C₁₋₃ heteroalkyl”). In some embodiments, a heteroalkyl group has 1 to 2 carbon atoms and 1 heteroatom, inclusive (“C₁₋₂ heteroalkyl”). In some embodiments, a heteroalkyl group has 1 carbon atom and 1 heteroatom, inclusive (“C₁ heteroalkyl”). In some embodiments, a heteroalkyl group has 2 to 6 carbon atoms and 1-3 heteroatoms, inclusive (“C₂₋₆ heteroalkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted C₁₋₁₀ alkyl. In certain embodiments, the heteroalkyl group is a substituted C₁₋₁₀ heteroalkyl.

As used herein, “alkynyl” refers to a radical of a straight-chain or branched hydrocarbon group having from 2 to 30 carbon atoms, one or more carbon-carbon triple bonds, and optionally one or more double bonds (“C₂₋₃₀ alkynyl”). In some embodiments, an alkynyl group has 2 to 20 carbon atoms (“C₂₋₂₀alkynyl”). In some embodiments, an alkynyl group has 2 to 10 carbon atoms (“C₂₋₁₀alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀alkynyl. In certain embodiments, the alkynyl group is a substituted C₂₋₁₀ alkynyl.

As used herein, “heteroalkynyl” refers to a radical of a straight-chain or branched hydrocarbon group having from 2 to 30 carbon atoms, one or more carbon-carbon triple bonds, optionally one or more double bonds, and which further comprises 1-10 heteroatoms independently selected from oxygen, nitrogen, and sulfur included within the parent chain (“C₂₋₃₀ heteroalkynyl”). In some embodiments, a heteroalkynyl group has 2 to 20 carbon atoms and 1-10 heteroatoms, inclusive (“C₂₋₂₀ heteroalkynyl”). In some embodiments, a heteroalkenyl group has 2 to 10 carbon atoms and 1-10 heteroatoms, inclusive (“C₂₋₁₀ heteroalkynyl”). In some embodiments, a heteroalkynyl group has 2 to 9 carbon atoms and 1-6 heteroatoms, inclusive (“C₂₋₉ heteroalkynyl”). In some embodiments, a heteroalkynyl group has 2 to 8 carbon atoms and 1-5 heteroatoms, inclusive (“C₂₋₈ heteroalkynyl”). In some embodiments, a heteroalkynyl group has 2 to 7 carbon atoms, and 1-4 heteroatoms, inclusive (“C₂₋₇ heteroalkynyl”). In some embodiments, a heteroalkynyl group has 2 to 6 carbon atoms and 1-3 heteroatoms, inclusive (“C₂₋₆ heteroalkynyl”). In some embodiments, a heteroalkynyl group has 2 to 5 carbon atoms and 1-2 heteroatoms, inclusive (“C₂₋₅ heteroalkynyl”). In some embodiments, a heteroalkynyl group has 2 to 4 carbon atoms and 1-2 heteroatoms, inclusive (“C₂₋₄ heteroalkynyl”). In some embodiments, a heteroalkynyl group has 2 to 3 carbon atoms and 1-2 heteroatoms, inclusive (“C₂₋₃ heteroalkynyl”). In some embodiments, a heteroalkynyl group has 2 carbon atoms and 1 heteroatom, inclusive (“C₂ heteroalkynyl”). In some embodiments, a heteroalkynyl group has 2 to 6 carbon atoms and 1-3 heteroatoms, inclusive (“C₂₋₆ heteroalkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted C₂₋₁₀ heteroalkynyl. In certain embodiments, the heteroalkynyl group is a substituted C₂₋₁₀ heteroalkynyl.

Generally, as used herein, substituent names which end in the suffix “-ene” refer to a biradical derived from the removal of an additional hydrogen atom from monoradical group as defined herein. Thus, for example, the monoradical alkyl is the biradical alkylene upon removal of an additional hydrogen atom from the alkyl. Likewise, alkenyl is alkenylene; alkynyl is alkynylene; heteroalkyl is heteroalkylene; heteroalkenyl is heteroalkenylene; heteroalkynyl is heteroalkynylene; carbocyclyl is carbocyclylene; heterocyclyl is heterocyclylene; aryl is arylene; and heteroaryl is heteroarylene.

Thus, as used herein, “alkylene” refers to a bivalent alkyl group.

Likewise, as used herein, “alkenylene” refers to a bivalent alkenyl group.

As used herein, “carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₀ cycloalkyl. In certain embodiments, the cycloalkyl group is a substituted C₃₋₁₀ cycloalkyl.

Note that in some embodiments, cycloalkyl rings have from about 3-10 carbon atoms in their ring structure where such rings are monocyclic, bicyclic, or polycyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

As used herein, “heterocyclyl”, “heterocyclic”, “heterocyclyl,” “heterocyclic radical,” and “heterocyclic ring” are used interchangeably and refer to a monocyclic, bicyclic or polycyclic ring moiety (e.g., 3-30 membered) that is saturated or partially unsaturated and has one or more heteroatom ring atoms. In some embodiments, a heteroatom is boron, nitrogen, oxygen, silicon, sulfur, or phosphorus. In some embodiments, a heteroatom is nitrogen, oxygen, silicon, sulfur, or phosphorus. In some embodiments, a heteroatom is nitrogen, oxygen, sulfur, or phosphorus. In some embodiments, a heteroatom is nitrogen, oxygen or sulfur. In some embodiments, a heterocyclyl group is a stable 5- to 7-membered monocyclic or 7- to 10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothienyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where a radical or point of attachment is on a heteroaliphatic ring. A heterocyclyl group may be monocyclic, bicyclic or polycyclic. The term “heterocyclylalkyl” refers to an alkyl group substituted by a heterocyclyl, wherein the alkyl and heterocyclyl portions independently are optionally substituted.

Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, and thiorenyl. Exemplary 4-membered heterocyclyl groups containing 1 heteroatom include, without limitation, azetidinyl, oxetanyl, and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include, without limitation, dioxolanyl, oxathiolanyl, and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, and dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl, and thiepanyl. Exemplary 8-membered heterocyclyl groups containing 1 heteroatom include, without limitation, azocanyl, oxecanyl, and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

As used herein, the term “aryl” used alone or as part of a larger moiety as in “aralkyl,” “aralkoxy,” “aryloxyalkyl,” etc. refers to monocyclic, bicyclic or polycyclic ring systems having a total of five to thirty ring members, wherein at least one ring in the system is aromatic. In some embodiments, an aryl group is a monocyclic, bicyclic or polycyclic ring system having a total of five to fourteen ring members, wherein at least one ring in the system is aromatic, and wherein each ring in the system contains 3 to 7 ring members. In some embodiments, an aryl group is a biaryl group. The term “aryl” may be used interchangeably with the term “aryl ring.” In certain embodiments of the present disclosure, “aryl” refers to an aromatic ring system which includes, but not limited to, phenyl, biphenyl, naphthyl, binaphthyl, anthracyl and the like. In some embodiments, 0, 1, 2, 3, or 4 atoms of each ring of an aryl ring are substituted by a substituent. In some embodiments, there are zero heteroatoms provided in the aromatic ring system (for example, “C₆₋₁₄ aryl”).

In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl group. In certain embodiments, the aryl group is a substituted C₆₋₁₄ aryl group. In some embodiments, also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like, where a radical or point of attachment is on an aryl ring.

“Aralkyl” is a subset of “alkyl” and refers to an alkyl group, as defined herein, substituted by an aryl group, as defined herein, wherein the point of attachment is on the alkyl moiety. The term “arylalkoxy” refers to an alkoxy subcultured with aryl.

The terms “heteroaryl” and “heteroar-,” used alone or as part of a larger moiety, e.g., “heteroaralkyl,” or “heteroaralkoxy,” refer to monocyclic, bicyclic or polycyclic ring systems having, for example, a total of five to thirty, e.g., 5, 6, 9, 10, 14, etc., ring members, wherein at least one ring in the system is aromatic and at least one aromatic ring atom is a heteroatom. In some embodiments, a heteroatom is nitrogen, oxygen or sulfur. In some embodiments, a heteroaryl group is a group having 5 to 10 ring atoms (i.e., monocyclic, bicyclic or polycyclic), in some embodiments 5, 6, 9, or 10 ring atoms. In some embodiments, a heteroaryl group has 6, 10, or 14 π electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. In some embodiments, a heteroaryl is a heterobiaryl group, such as bipyridyl and the like. The terms “heteroaryl” and “heteroar-”, as used herein, also include groups in which a heteroaromatic ring is fused to one or more aryl, cycloaliphatic, or heterocyclyl rings, where a radical or point of attachment is on a heteroaromatic ring. Non-limiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be monocyclic, bicyclic or polycyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl group, wherein the alkyl and heteroaryl portions independently are optionally substituted.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

The term “heteroalkyl” is given its ordinary meaning in the art and refers to an alkyl group in which one or more carbon atoms is replaced with a heteroatom (e.g., oxygen, nitrogen, sulfur, silicon, phosphorus, and the like), or substituted by a heteroaryl group, wherein the point of attachment is on the alkyl moiety. Examples of heteroalkyl groups include, but are not limited to, alkoxy, poly(ethylene glycol)-, alkyl-substituted amino, tetrahydrofuranyl, piperidinyl, morpholinyl, etc

The term “heteroatom” means an atom that is not carbon and is not hydrogen. In some embodiments, a heteroatom is oxygen, sulfur, nitrogen, phosphorus, boron or silicon (including any oxidized form of nitrogen, sulfur, phosphorus, or silicon; the quaternized form of any basic nitrogen or a substitutable nitrogen of a heterocyclic ring (for example, N as in 3,4-dihydro-2H-pyrrolyl), NH (as in pyrrolidinyl) or NR⁺ (as in N-substituted pyrrolidinyl); etc.). In some embodiments, a heteroatom is boron, nitrogen, oxygen, silicon, sulfur, or phosphorus. In some embodiments, a heteroatom is nitrogen, oxygen, silicon, sulfur, or phosphorus. In some embodiments, a heteroatom is nitrogen, oxygen, sulfur, or phosphorus. In some embodiments, a heteroatom is nitrogen, oxygen or sulfur.

As used herein, the term “partially unsaturated” refers to a group that includes at least one double or triple bond. The term “partially unsaturated” is intended to encompass rings having multiple sites of unsaturation, but is not intended to include aromatic groups (e.g., aryl or heteroaryl moieties) as herein defined.

As used herein, the term “saturated” refers to a group that does not contain a double or triple bond, i.e., contains all single bonds.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present disclosure contemplates any and all such combinations in order to arrive at a stable compound. For purposes of the present disclosure, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, C(═O)N(R^{bb})₂, —OC(═O)N(R^{bb})₂, —NR^{bb}C(═O)R^{aa}, —NR^{bb}CO₂R^{aa}, —NP^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —N^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃, —C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂—NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀alkynyl, C₃₋₁₄ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, —NNR^{bb}C(═O)R^{aa}, —NNR^{bb}C(═O)OR^{aa}, ═NNR^{bb}S(═O)₂R^{aa}, ═NR^{bb}, or ═NOR^{cc};
each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{cc}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S;
each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups;
each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and
each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S; wherein X⁻ is a counterion.

As used herein, the term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein.

As used herein, the term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═O)SR^{aa}, —SC(═O)OR^{aa}, and —SC(═O)R^{aa}, wherein R^{aa}, and R^{cc} are as defined herein.

As used herein, the term, “amino” refers to the group —NH₂. The term “substituted amino,” by extension, refers to a monosubstituted amino, a disubstituted amino, or a trisubstituted amino, as defined herein.

As used herein, the term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(NR^{bb})₂, wherein R^{aa}, R^{bb}, and R^{cc} are as defined herein, and wherein R^{bb} of the group —NH(R^{bb}) is not hydrogen.

As used herein, the term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(NR^{bb})₂ wherein R^{aa}, R^{bb}, and R^{cc} are as defined herein, with the proviso that the nitrogen atom directly attached to the parent molecule is not substituted with hydrogen.

As used herein, the term “trisubstituted amino” or a “quaternary amino salt” or a “quaternary salt” refers to a nitrogen atom covalently attached to four groups such that the nitrogen is cationic, wherein the cationic nitrogen atom is further complexed with an anionic counterion, e.g., such as groups of the Formula —N(R^{bb})₃⁺X⁻ and —N(R^{bb})₂—⁺X⁻, wherein R^{bb} and X⁻ are as defined herein.

As used herein, a “counterion” or “anionic counterion” is a negatively charged group associated with a cationic quaternary amino group in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

As used herein, the term “sulfonyl” refers to a group selected from —SO₂N(R^{bb})₂, —SO₂R^{aa}, and —SO₂OR^{aa}, wherein R^{aa} and R^{bb} are as defined herein.

As used herein, the term “sulfinyl” refers to the group —S(═O)R^{aa}, wherein R^{aa} is as defined herein.

As used herein, the term “acyl” refers a group wherein the carbon directly attached to the parent molecule is sp² hybridized, and is substituted with an oxygen, nitrogen or sulfur atom. In some embodiments, the acyl group thus contains a double bonded oxygen atom and an alkyl group, and has the general formula —C(═O)-alkyl (where the alkyl can be, for example, a unsubstituted or a substituted alkyl (e.g., C₁₋₁₀ alkyl such as an acetyl)). Non-limiting examples of acyl groups include ketones (—C(═O)-alkyl, such as —C(═OR)C), carboxylic acids (—CO₂H), aldehydes (—CHO), esters (—CO₂-alkyl, such as CO₂C, thioesters (—C(═O)S-alkyl, —C(═S)S-alkyl), amides, thioamides, and imines.

As used herein, the term “azido” refers to a group of the formula: —N₃.

As used herein, the term “cyano” refers to a group of the formula: —CN.

As used herein, the term “isocyano” refers to a group of the formula: —NC.

As used herein, the term “nitro” refers to a group of the formula: —NO₂.

As used herein, the term “halo” or “halogen” refers to fluorine (fluoro, —F), chlorine (chloro, —Cl), bromine (bromo, —Br), or iodine (iodo, —I).

As used herein, the term “oxo” refers to a group of the formula: ═O.

As used herein, the term “thiooxo” refers to a group of the formula: ═S.

As used herein, the term “imino” refers to a group of the formula: ═N(R^{b}).

As used herein, the term “silyl” refers to the group —Si(R^{aa})₃, wherein R^{aa} is as defined herein.

Nitrogen atoms can be substituted or unsubstituted as valency permits, and include primary, secondary, tertiary, and quarternary nitrogen atoms. Exemplary nitrogen atom substitutents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an amino protecting group (also referred to herein as a “nitrogen protecting group”). Amino protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 Rad groups, and wherein R^{aa}, R^{bb}, R^{cc}, and R^{dd} are as defined herein. Amino protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, amino protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide and o-(benzoyloxymethyl)benzamide.

Amino protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Amino protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pme), methanesulfonamide (Ms), β-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other amino protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is a hydroxyl protecting group (also referred to herein as an “oxygen protecting group”). Hydroxyl protecting groups include, but are not limited to, —R^{aa}, —N(R^{bb})₂, —C(═O)SR^{aa}, —C(═O)R^{aa}, —CO₂R^{aa}, C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Hydroxyl protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMB13M), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), isobutyl carbonate, vinyl carbonate, allyl carbonate, t-butyl carbonate (BOC), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

A “thiol protecting group” is well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of protected thiol groups further include, but are not limited to, thioesters, carbonates, sulfonates allyl thioethers, thioethers, silyl thioethers, alkyl thioethers, arylalkyl thioethers, and alkyloxyalkyl thioethers. Examples of ester groups include formates, acetates, proprionates, pentanoates, crotonates, and benzoates. Specific examples of ester groups include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetate), crotonate, 4-methoxy-crotonate, benzoate, p-benylbenzoate, 2,4,6-trimethylbenzoate. Examples of carbonates include 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl carbonate. Examples of silyl groups include trimethylsilyl, triethylsilyl, t-butyldimethylsilyl, t-butyldiphenylsilyl, triisopropylsilyl ether, and other trialkylsilyl ethers. Examples of alkyl groups include methyl, benzyl, p-methoxybenzyl, 3,4-dimethoxybenzyl, trityl, t-butyl, and allyl ether, or derivatives thereof. Examples of arylalkyl groups include benzyl, p-methoxybenzyl (MPM), 3,4-dimethoxybenzyl, 0-nitrobenzyl, p-nitrobenzyl, p-halobenzyl, 2,6-dichlorobenzyl, p-cyanobenzyl, 2- and 4-picolyl ethers.

The term “amino acid” is used interchangeably with “amino acid residue” and “amino acid residue analog”, and refers to a molecule containing both an amino group and a carboxyl group. Amino acids include alpha-amino acids and beta-amino acids, the structures of which are depicted below. In certain embodiments, the amino acid is an alpha-amino acid. In certain embodiments, the amino acid is an unnatural amino acid. In certain embodiments, the amino acid is a natural amino acid. In certain embodiments, the amino acid is an unnatural amino acid.

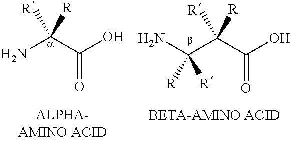

Exemplary amino acids include, without limitation, natural alpha-amino acids such as D- and L-isomers of the 20 common naturally occurring alpha amino acids found in peptides, unnatural alpha-amino acids, natural beta-amino acids (e.g., beta-alanine), and unnnatural beta-amino acids. Amino acids used in the construction of peptides of the present disclosure may be prepared by organic synthesis, or obtained by other routes, such as, for example, degradation of or isolation from a natural source. Amino acids may be commercially available or may be synthesized.

In certain embodiments, each instance of an amino acid (or an amino acid residue analog) is, independently, a natural L-amino acid as provided in Table A, or an unnatural amino acid as provided in Tables B, C, D, and/or E.

TABLE A

| Exemplary natural | | |
|---|---|---|
| alpha-amino acids | R | R′ |
| L-Alanine (A) | —CH₃ | —H |
| L-Arginine (R) | —CH₂CH₂CH₂—NHC(═NH)NH₂ | —H |
| L-Asparagine (N) | —CH₂C(═O)NH₂ | —H |
| L-Aspartic acid (D) | —CH₂CO₂H | —H |
| L-Cysteine (C) | —CH₂SH | —H |
| L-Glutamic acid (E) | —CH₂CH₂CO₂H | —H |
| L-Glutamine (Q) | —CH₂CH₂C(═O)NH₂ | —H |
| Glycine (G) | —H | —H |
| L-Histidine (H) | CH₂-2-(1H-imidazole) | —H |
| L-Isoleucine (I) | -sec-butyl | —H |
| L-Leucine (L) | -iso-butyl | —H |
| L-Lysine (K) | —CH₂CH₂CH₂CH₂NH₂ | —H |
| L-Methionine (M) | —CH₂CH₂SCH₃ | —H |
| L-Phenylalanine (F) | —CH₂Ph | —H |
| L-Proline (P) | -2-(pyrrolidine) | —H |
| L-Serine (S) | —CH₂OH | —H |
| L-Threonine (T) | —CH₂CH(OH)(CH₃) | —H |
| L-Tryptophan (W) | —CH₂-3-(1H-indole) | —H |
| L-Tyrosine (Y) | —CH₂-(p-hydroxyphenyl) | —H |
| L-Valine (V) | -isopropyl | —H |

TABLE B

| Exemplary unnatural | | |
|---|---|---|
| alpha-amino acids | R | R′ |
| D-Alanine | —H | —CH₃ |
| D-Arginine | —H | —CH₂CH₂CH₂—NHC(═NH)NH₂ |
| D-Asparagine | —H | —CH₂C(═O)NH₂ |
| D-Aspartic acid | —H | —CH₂CO₂H |
| D-Cysteine | —H | —CH₂SH |
| D-Glutamic acid | —H | —CH₂CH₂CO₂H |
| D-Glutamine | —H | —CH₂CH₂C(═O)NH₂ |
| D-Histidine | —H | —CH₂-2-(1H-imidazole) |
| D-Isoleucine | —H | -sec-butyl |
| D-Leucine | —H | -iso-butyl |
| D-Lysine | —H | —CH₂CH₂CH₂CH₂NH₂ |
| D-Methionine | —H | —CH₂CH₂SCH₃ |
| D-Phenylalanine | —H | —CH₂Ph |
| D-Proline | —H | -2-(pyrrolidine) |
| D-Serine | —H | —CH₂OH |
| D-Threonine | —H | —CH₂CH(OH)(CH₃) |
| D-Tryptophan | —H | —CH₂-3-(1H-indole) |
| D-Tyrosine | —H | —CH₂-(p-hydroxyphenyl) |
| D-Valine | —H | -isopropyl |
| Di-vinyl | —CH═CH₂ | —CH=CH₂ |

TABLE C

| Exemplary unnatural | | |
|---|---|---|
| alpha-amino acids | R and R′ are equal to: | |
| α-methyl-Alanine (Aib, 2- | —CH₃ | —CH₃ |
| amino-2-methylpropanoic acid) | | |
| α-methyl-Arginine | —CH₃ | —CH₂CH₂CH₂—NHC(═NH)NH₂ |
| α-methyl-Asparagine | —CH₃ | —CH₂C(═O)NH₂ |
| α-methyl-Aspartic acid | —CH₃ | —CH₂CO₂H |
| α-methyl-Cysteine | —CH₃ | —CH₂SH |
| α-methyl-Glutamic acid | —CH₃ | —CH₂CH₂CO₂H |
| α-methyl-Glutamine | —CH₃ | —CH₂CH₂C(═O)NH₂ |
| α-methyl-Histidine | —CH₃ | —CH₂-2-(1H-imidazole) |
| α-methyl-Isoleucine | —CH₃ | -sec-butyl |
| α-methyl-Leucine | —CH₃ | -iso-butyl |
| α-methyl-Lysine | —CH₃ | —CH₂CH₂CH₂CH₂NH₂ |
| α-methyl-Methionine | —CH₃ | —CH₂CH₂SCH₃ |
| α-methyl-Phenylalanine | —CH₃ | —CH₂Ph |
| α-methyl-Proline | —CH₃ | -2-(pyrrolidine) |
| α-methyl-Serine | —CH₃ | —CH₂OH |
| α-methyl-Threonine | —CH₃ | —CH₂CH(OH)(CH₃) |
| α-methyl-Tryptophan | —CH₃ | —CH₂-3-(1H-indole) |
| α-methyl-Tyrosine | —CH₃ | —CH₂-(p-hydroxyphenyl) |
| α-methyl-Valine | —CH₃ | -isopropyl |
| Di-vinyl | —CH═CH₂ | —CH=CH₂ |
| Norleucine | —H | —CH₂CH₂CH₂CH₃ |

TABLE D

| | R and R′ is independently equal to |
|---|---|
| Exemplary unnatural alpha-amino acids | hydrogen or —CH₃, and the group: |
| Terminally unsaturated alpha-amino | —(CH₂)_{g}—S—(CH₂)_{g}CH═CH₂, |
| acids and bis alpha-amino acids (e.g., | —(CH₂)_{g}—O—(CH₂)_{g}CH═CH₂, |
| modified cysteine, modified lysine, | —(CH₂)_{g}—NH—(CH₂)_{g}CH═CH₂, |
| modified tryptophan, modified serine, | —(CH₂)_{g}—(C═O)—S—(CH₂)_{g}CH═CH₂, |
| modified threonine, modified proline, | —(CH₂)_{g}—(C═O)—O—(CH₂)_{g}CH═CH₂, |
| modified histidine, modified alanine, | —(CH₂)_{g}—(C═O)—NH—(CH₂)_{g}CH═CH₂, |
| and the like). | —CH₂CH₂CH₂CH₂—NH—(CH₂)_{g}CH═CH₂, |
| | —(C₆H₅)-p-O—(CH₂)_{g}CH═CH₂, |
| | —CH(CH₃)—O—(CH₂)_{g}CH═CH₂, |
| | —CH₂CH(—O—CH═CH₂)(CH₃), |
| | -histidine-N((CH₂)_{g}CH═CH₂), |
| | -tryptophan-N((CH₂)_{g}CH═CH₂), and |
| | —(CH₂)_{g}(CH═CH₂), |
| | wherein: |
| | each instance of g is, independently, 0 to 10. |

TABLE E

| Other unnatural alpha-amino acids | R and R′ are equal to: | |
|---|---|---|
| 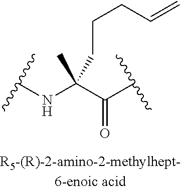 [SMILES: *N[C@](C)(CCCC=C)C(*)=O] | —CH₃ | —(CH₂)₃CH═CH₂ |
| 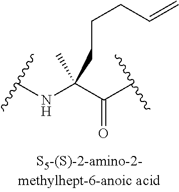 [SMILES: *N[C@@](C)(CCCC=C)C(*)=O] | | |
| 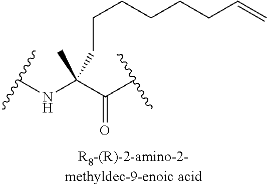 [SMILES: *N[C@](C)(CCCCCCC=C)C(*)=O] | —CH₃ | —(CH₂)₆CH═CH₂ |
| 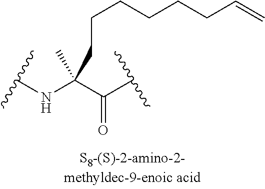 [SMILES: *N[C@@](C)(CCCCCCC=C)C(*)=O] | | |
| 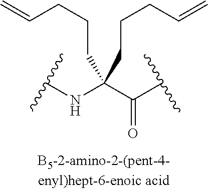 | —(CH₂)₃CH═CH₂ | —(CH₂)₃CH═CH₂ |

In some embodiments, an amino acid residue is suitable for stapling, e.g., via olefin metathesis:

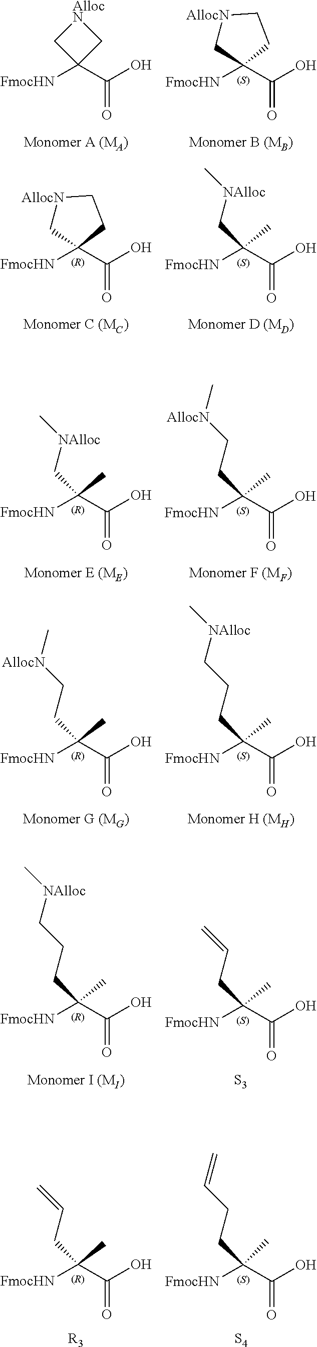

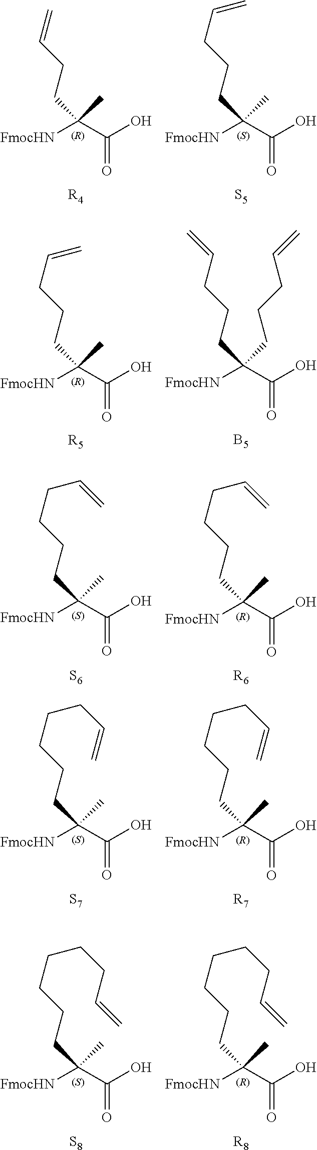

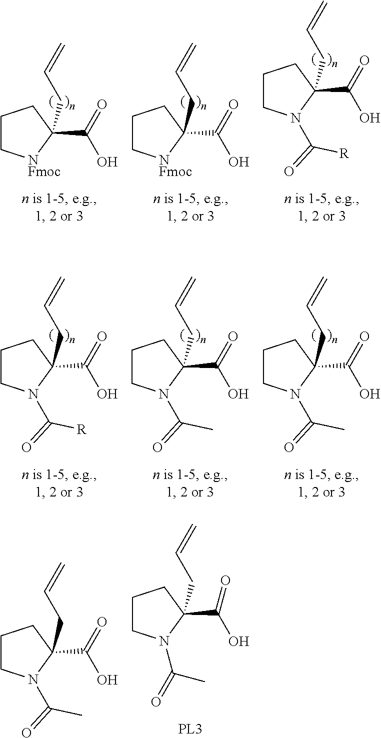

There are many known unnatural amino acids any of which may be included in the peptides of the present disclosure. See, for example, S. Hunt, The Non-Protein Amino Acids: In Chemistry and Biochemistry of the Amino Acids, edited by G. C. Barrett, Chapman and Hall, 1985. Some additional examples of unnatural amino acids are 4-hydroxyproline, desmosine, gamma-aminobutyric acid, beta-cyanoalanine, norvaline, 4-(E)-butenyl-4(R)-methyl-N-methyl-L-threonine, N-methyl-L-leucine, 1-amino-cyclopropanecarboxylic acid, 1-amino-2-phenyl-cyclopropanecarboxylic acid, 1-amino-cyclobutanecarboxylic acid, 4-amino-cyclopentenecarboxylic acid, 3-amino-cyclohexanecarboxylic acid, 4-piperidylacetic acid, 4-amino-1-methylpyrrole-2-carboxylic acid, 2,4-diaminobutyric acid, 2,3-diaminopropionic acid, 2,4-diaminobutyric acid, 2-aminoheptanedioic acid, 4-(aminomethyl)benzoic acid, 4-aminobenzoic acid, ortho-, meta- and para-substituted phenylalanines (e.g., substituted with —C(═O)C₆H₅; —CF₃; —CN; -halo; —NO₂; —CH₃), disubstituted phenylalanines, substituted tyrosines (e.g., further substituted with —C(═O)C₆H₅; —CF₃; —CN; -halo; —NO₂; —CH₃), and statine. Furthermore, the amino acids for use in the present disclosure may be derivatized to include amino acid residues that are hydroxylated, phosphorylated, sulfonated, acylated, alkylated, farnesylated, geryanylated, and/or glycosylated.

A “peptide” or “polypeptide” comprises a polymer of amino acid residues linked together by peptide (amide) bonds. The term(s), as used herein, refers to proteins, polypeptides, and peptide of any size, structure, or function. The term(s), as used herein, include stapled, unstapled, stitched, and unstitched polypeptides. Typically, a peptide or polypeptide will be at least three amino acids long. A peptide or polypeptide may refer to an individual protein or a collection of proteins. One or more of the amino acids in a peptide or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification. A peptide or polypeptide may also be a single molecule or may be a multi-molecular complex, such as a protein. A peptide or polypeptide may be just a fragment of a naturally occurring protein or peptide. A peptide or polypeptide may be naturally occurring, recombinant, or synthetic, or any combination thereof.

In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, a polypeptide may comprise or consist of natural amino acids, non-natural amino acids, or both. In some embodiments, a polypeptide may comprise or consist of only natural amino acids or only non-natural amino acids. In some embodiments, a polypeptide may comprise D-amino acids, L-amino acids, or both. In some embodiments, a polypeptide may comprise only D-amino acids. In some embodiments, a polypeptide may comprise only L-amino acids. In some embodiments, a polypeptide may include one or more pendant groups or other modifications, e.g., modifying or attached to one or more amino acid side chains, at the polypeptide's N-terminus, at the polypeptide's C-terminus, or any combination thereof. In some embodiments, such pendant groups or modifications may be selected from the group consisting of acetylation, amidation, lipidation, methylation, pegylation, etc., including combinations thereof. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may be or comprise a stapled polypeptide. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments be or comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a relevant polypeptide may comprise or consist of a fragment of a parent polypeptide. In some embodiments, a useful polypeptide as may comprise or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide.

### DETAILED DESCRIPTION OF THE CERTAIN EMBODIMENTS

In some embodiments, the present disclosure provides a peptide comprising an amino acid residue having the structure of P-I:

—N(R^{a1})CH(R^{a2})—C(O)—, (P-I)

or a salt form thereof, wherein:

R^{a1} and R^{a2} are taken together with their intervening atoms to form Ring A;
Ring A is a substituted 3-10 membered saturated or partially unsaturated ring having 0-3 heteroatoms in addition to the nitrogen to which R^{a1} is attached, wherein at least one substituent of the ring is —K—R^{a3}, or —K—, wherein K is connected to the side chain or backbone carbon of a second amino acid residue optionally through a linker S^{p};
each K is independently a covalent bond, or an optionally substituted C₁₋₂₀ aliphatic or heteroaliphatic chain having 1-6 heteroatoms, wherein one or more methylene unit is optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each R^{a3} is independently an optionally substituted group selected from —CH═CH₂ and —C≡CH;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
S^{p} is —S^{p1}—S^{p2}—S^{p3}—, wherein S^{p1} is bonded to K and S^{p3} is bonded to a side chain or backbone carbon of a second amino acid residue;
each of S^{p1}, S^{p2}, and S^{p3} is independently S^{L};
each S^{L} is independently a bond, a substituted or unsubstituted C₁₋₁₀ alkane, a substituted or unsubstituted C₁₋₁₀ alkylene, or an optionally substituted, bivalent C₁-C₂₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each R′ is independently —R, —C(O)R, —CO₂R, or —SO₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.
   In some embodiments, each heteroatom in a structure of the present disclosure is independently selected from nitrogen, oxygen and sulfur. In some embodiments, Ring A is a monocyclic. In some embodiments, Ring A is saturated. In some embodiments, Ring A is partially unsaturated. In some embodiments, —K— is bonded to —CH— to which R^{a2} is attached to (replacing the H). In some embodiments, R^{a3} is —CH═CH₂ and —C≡CH. In some embodiments, R^{a3} is optionally substituted —CH═CH₂. In some embodiments, R^{a3} is —CH═CH₂. In some embodiments, R^{a3} is optionally substituted —C≡CH. In some embodiments, R^{a3} is —C≡CH. In some embodiments, K is optionally substituted bivalent C₁₋₁₀ alphatic. In some embodiments, K is optionally substituted bivalent C₁₋₁₀ alkylene. In some embodiments, K is linear bivalent C₁₋₁₀ alphatic. In some embodiments, K is linear bivalent C₁₋₁₀ alkylene. In some embodiments, K is optionally substituted bivalent C₁₋₁₀ heteroalphatic having 1-4 heteroatoms. In some embodiments, K is optionally substituted bivalent C₁₋₁₀ heteroalkylene having 1-4 heteroatoms. In some embodiments, K is linear bivalent C₁₋₁₀ heteroalphatic having 1-4 heteroatoms. In some embodiments, K is linear bivalent C₁₋₁₀ heteroalkylene having 1-4 heteroatoms. In some embodiments, a provided peptide comprises a residue having the structure of formula P-I:

—C(O)—N(R^{a1})CH(R^{a2})—C(O)— (P-II)

or a salt form thereof, wherein each variable is independently as described herein. In some embodiments, a provided peptide comprises a residue having the structure of formula P-III:

R^{a}—N(R^{a1})CH(R^{a2})—C(O)— (P-III)

or a salt form thereof, wherein R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group selected from, or one or more combinations of, substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; and substituted or unsubstituted heteroarylene; or R^{a} is or comprises a peptide moiety; and each other variable is independently as described herein. In some embodiments, R^{a} is optionally substituted acyl. In some embodiments, R^{a} is R—C(O)—. In some embodiments, R^{a} is acetyl. In some embodiments, an amino acid has the structure of R^{a}—N(R^{a1})CH(R^{a2})—C(O)OH or a salt thereof. In some embodiments, R^{a} is or comprises a peptide moiety. In some embodiments, R^{a3} forms a metathesis product connection, e.g., —CH═CH—, with another double or triple bond of a peptide to form a staple. In some embodiments, Ring A is substituted with —K—R^{a3} (e.g., in certain unstapled peptides, amino acids, etc.). In some embodiments, Ring A is substituted with K, —K—, wherein K is connected to the side chain or backbone carbon of a second amino acid residue optionally through a linker S^{p}.

Various provided compound in the present disclosure may have R^{a}. In some embodiments, R^{a} is H. In some embodiments, R^{a} is optionally substituted acyl. In some embodiments, R^{a} is a suitable amino protecting group. In some embodiments, R^{a} is Fmoc. In some embodiments, R^{a} is t-Boc. As described above, in certain embodiments R^{a} is R—C(O)—. In some embodiments, R^{a} is acetyl.

In some embodiments, a residue of formula P-I has the structure of

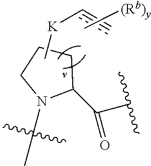

or salt form thereof, wherein each variable is independently as described herein.

In some embodiments, the present disclosure provides a peptide comprising an amino acid residue B¹, wherein:
B¹ is B or B′;
B is

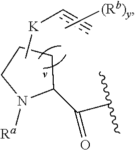

or a salt form thereof,
B′ is

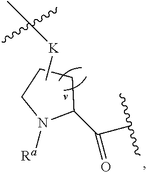

or a salt form thereof,
wherein:
v is 1 or 2;
K is a covalent bond, or an substituted or unsubstituted bivalent group selected from a bivalent aliphatic group, alkylene, alkenylene, alkynylene, a bivalent heteroaliphatic group, heteroalkylene, heteroalkenylene, heteroalkynylene, heterocyclene, carbocyclene, arylene, and heteroarylene, and when B¹ is B′, K is connected to the side chain or backbone carbon of a second amino acid residue optionally through a linker S^{p};
R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group selected from, or one or more combinations of, substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; and substituted or unsubstituted heteroarylene;
each instance of R^{b}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
y is 0, 1, 2, or 3; [symbol]
each instance of [symbol] independently represents a single bond, a double bond or a triple bond;
S^{p} is —S^{p1}—S^{p2}—S^{p3}—, wherein S^{p1} is bonded to K and S^{p3} is bonded to a side chain or backbone carbon of a second amino acid residue;
each of S^{p1}, S^{p2}, and S^{p3} is independently S^{L};
each S^{L} is independently a bond, a substituted or unsubstituted C₁₋₁₀ alkane, a substituted or unsubstituted C₁₋₁₀ alkylene, or an optionally substituted, bivalent C₁-C₂₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In some embodiments, the present disclosure provides a peptide comprising an amino acid residue B¹, wherein R^{a} is or comprises a peptide moiety, and each other variable is independently as described herein. In some embodiments, R^{a} is R′—[X]d-, wherein d is 1, 2, 3, 4, 5, 6, 7, 8, 9 10, 11, 12, 13, 14, 15, 16, 17, 18, 19, or 20. In some embodiments, R′ is R—C(O)—. In some embodiments, R is optionally substituted C₁₋₁₀ aliphatic. In some embodiments, R is optionally substituted C₁₋₁₀ alkyl. In some embodiments, R is methyl.

In some embodiments, B¹ is B as described herein. In some embodiments, B¹ is B, and a provided peptide comprises a second amino acid residue whose side chain comprises a double bond or a triple bond. In some embodiments, a second amino acid residue comprises a double bond. In some embodiments, a second amino acid residue comprises a terminal double bond. Among other things, under suitable conditions B¹ and a second amino acid residue may form a staple, e.g., via metathesis. In some embodiments, B¹ is B′ as described herein. In some embodiments, a second amino acid is J as described herein. In some embodiments, a provided peptide comprises one or more Z as described herein. Suitable positions for J and/or one or more Z residues include those described in sequences herein. In some embodiments, a second amino acid residue is J′ as described herein (with S^{p} included). In some embodiments, a staple is between two residues at position i and i+3, wherein i is the position of B¹. In some embodiments, J or J′ is at position i+3 while B¹ is at position i. In some embodiments, i is 1.

In some embodiments, a group, e.g., a monovalent group such as aliphatic, alkyl, alkenyl, alkynyl, heteroaliphatic, heteroalkyl, aryl, heteroaryl, acyl, etc. (e.g., those recited in R^{a}, R^{b}, R^{a1}, R^{a2}, R^{a3}, etc.), or a bivalent group such as a bivalent alphatic or heteroaliphatic group, alkylene, alkenylene, alkynylene, heteroalkylene, heteroalkenylene, heteroalkynylene, heterocyclene, carbocyclene, arylene, and heteroarylene, etc. (e.g., those recited in K, S^{p}, S^{p1}, S^{p2}, S^{p3}, S^{L}, etc.) is a C₁₋₂, C₁₋₄, C₁₋₆, C₁₋₈, C₁₋₁₀, C₁₋₁₅, or C₁₋₂₀ and having 0-10 heteroatoms and is further optionally substituted. In some embodiments, each heteroatom is independently selected from nitrogen, oxygen and sulfur.

In some embodiments, S^{p} is bonded to a backbone carbon (e.g., alpha-carbon) of a second amino acid residue. In some embodiments, S^{p} is bonded to a side chain of a second amino acid residue (e.g., to L₁). In some embodiments, K is —CH₂—. In some embodiments, K is —CH₂CH₂—. In some embodiments, K is —CH₂CH₂CH₂—. In some embodiments, S^{p} is —CH═CH—CH₂—. In some embodiments, S^{p} is —CH═CH—CH₂CH₂—. In some embodiments, S^{p} is —CH═CH—CH₂CH₂CH₂—. In some embodiments, K is —CH₂—, S^{p} is —CH═CH—CH₂CH₂CH₂—, and S is bonded to an alpha carbon of a second amino acid residue. In some embodiments, the double bond in S^{p} is cis. In some embodiments, the double bond in S^{p} is trans.

In a first aspect, the present disclosure provides a peptide, wherein the peptide is or comprises:

(SEQ ID NO: 1)
B-X²-Z-J-X⁵-X⁶-Z-X⁸-X⁹-X¹⁰-X¹¹-X¹²-X¹³,

or a salt thereof, wherein:
B is

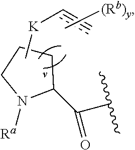

or a salt or a stereoisomeric form thereof, wherein:
v is 1 or 2;
K is a covalent bond, or an substituted or unsubstituted bivalent group selected from a bivalent aliphatic group, alkylene, alkenylene, alkynylene, a bivalent heteroaliphatic group, heteroalkylene, heteroalkenylene, heteroalkynylene, heterocyclene, carbocyclene, arylene, and heteroarylene;
R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group selected from, or one or more combinations of, substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; and substituted or unsubstituted heteroarylene;
each instance of R^{b}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
y is 0, 1, 2, or 3; and
each instance of [symbol] independently represents a single bond, a double bond or a triple bond;
J is

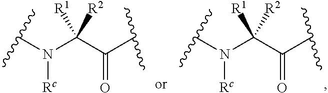

or a salt or a stereoisomeric form thereof, wherein:
each instance of R¹ and R² is independently hydrogen; substituted or unsubstituted aliphatic; substituted or unsubstituted alkylene; substituted or unsubstituted alkynylene; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; or halo; and
each instance of R^{c}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
each instance of Z is independently an amino acid residue which comprises an optionally substituted C₄₋₆ (e.g., C₄, C₅, or C₆) aliphatic side chain, or a leucine amino acid residue or a homolog thereof, such as, for example, a residue selected from the group consisting of a leucine amino acid residue, an isoleucine amino acid residue, a homoleucine amino acid residue, an alloisoleucine amino acid residue, a norleucine amino acid residue, and a tert-leucine amino acid residue, wherein the homolog may be a D stereoisomer or an L stereoisomer;
each of X², X⁵, X⁶, X⁸, X⁹, X¹⁰, X¹¹, X¹², and X¹³ is independently an amino acid residue which may be a D stereoisomer or an L stereoisomer; and
each of X⁹, X¹⁰, X¹¹, X¹², and X¹³ is optionally present.

In some embodiments, X⁶ is Z.

In some embodiments, the present disclosure provides a peptide, wherein the peptide is or comprises:

(SEQ ID NO: 1)
B-X²-Z-J-X⁵-X⁶-Z-X⁸-X⁹-X¹⁰-X¹¹-X¹²-X¹³,

or a salt thereof, wherein R^{a} is or comprises a peptide moiety, and each other variable is independently as described herein. In some embodiments, R^{a} is R′—[X]d-. In some embodiments, R′ is R—C(O)—. In some embodiments, R is optionally substituted C₁₋₁₀ aliphatic. In some embodiments, R is optionally substituted C₁₋₁₀ alkyl. In some embodiments, R is methyl.

In a first aspect, the present disclosure provides a peptide, wherein the peptide is or comprises:

B′-X²-Z-J′-X⁵-X⁶-Z-X⁸-X⁹-X¹⁰-X¹¹-X¹²-X¹³

or a salt thereof, wherein:
B′ is

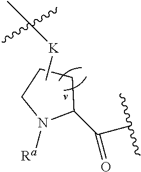

or a salt or a stereoisomeric form thereof, wherein:
v is 1 or 2;
K is a covalent bond, or an substituted or unsubstituted bivalent group selected from a bivalent aliphatic group, alkylene, alkenylene, alkynylene, a bivalent heteroaliphatic group, heteroalkylene, heteroalkenylene, heteroalkynylene, heterocyclene, carbocyclene, arylene, and heteroarylene;
R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group selected from, or one or more combinations of, substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; and substituted or unsubstituted heteroarylene;
J′ is

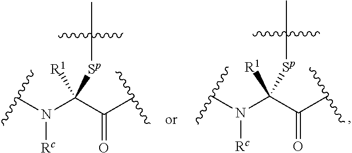

or a salt or a stereoisomeric form thereof,
each S^{p} is independently —S^{p1}—S^{p2}—S^{p3}—, wherein S^{p1} is bonded to K;
each of S^{p1}, S^{p2}, and S^{p3} is independently S^{L};
each S^{L} is independently a bond, a substituted or unsubstituted C₁₋₁₀ alkane, a substituted or unsubstituted C₁₋₁₀ alkylene, or an optionally substituted, bivalent C₁-C₂₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or two R groups are optionally and independently taken together to form a covalent bond, or: two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon;
each instance of R¹ is independently hydrogen; substituted or unsubstituted aliphatic; substituted or unsubstituted alkylene; substituted or unsubstituted alkynylene; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; or halo;
each instance of R^{c}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
each instance of Z is independently an amino acid residue which comprises an optionally substituted C₄₋₆ (e.g., C₄, C₅, or C₆) aliphatic side chain, or a leucine amino acid residue or a homolog thereof, such as, for example, a residue selected from the group consisting of a leucine amino acid residue, an isoleucine amino acid residue, a homoleucine amino acid residue, an alloisoleucine amino acid residue, a norleucine amino acid residue, and a tert-leucine amino acid residue, wherein the homolog may be a D stereoisomer or an L stereoisomer;
each of X², X⁵, X⁶, X⁸, X⁹, X¹⁰, X¹¹, X², and X¹³ is independently an amino acid residue which may be a D stereoisomer or an L stereoisomer; and
each of X⁹, X¹⁰, X¹¹, X¹², and X¹³ is optionally present.

In some embodiments, X⁶ is Z.

In some embodiments, the present disclosure provides a peptide, wherein the peptide is or comprises:

B′-X²-Z-J′-X⁵-X⁶-Z-X⁸-X⁹-X¹⁰-X¹¹-X¹²-X¹³,

or a salt thereof, wherein R^{a} is or comprises a peptide moiety, and each other variable is independently as described herein. In some embodiments, R^{a} is R′—[X]d-. In some embodiments, R′ is R—C(O)—. In some embodiments, R is optionally substituted C₁₋₁₀ aliphatic. In some embodiments, R is optionally substituted C₁₋₁₀ alkyl. In some embodiments, R is methyl.

In another aspect, the present disclosure provides a peptide, wherein the peptide is or comprises:

(SEQ ID NO: 2)
B-Z-X³-J-X⁵-Z-X⁷-X⁸-X⁹-X¹⁰-X¹¹-X¹²-X¹³,

or a salt thereof, wherein:
B is

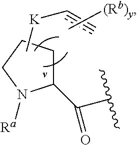

or a salt or a stereoisomeric form thereof, wherein:
v is 1 or 2;
K is a covalent bond, or an substituted or unsubstituted bivalent group selected from a bivalent aliphatic group, alkylene, alkenylene, alkynylene, a bivalent heteroaliphatic group, heteroalkylene, heteroalkenylene, heteroalkynylene, heterocyclene, carbocyclene, arylene, and heteroarylene;
R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group selected from, or one or more combinations of, substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; and substituted or unsubstituted heteroarylene;
each instance of R^{b}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
y is 0, 1, 2, or 3; and
each instance of [symbol] independently represents a single bond, a double bond or a triple bond;
J is

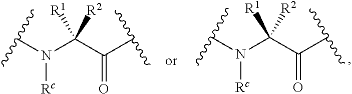

or a salt or a stereoisomeric form thereof, wherein:
each instance of R¹ and R² is independently hydrogen; substituted or unsubstituted aliphatic; substituted or unsubstituted alkylene; substituted or unsubstituted alkynylene; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; or halo; and
each instance of R^{c}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
each instance of Z is independently an amino acid residue which comprises an optionally substituted C₄₋₆ (e.g., C₄, C₅, or C₆) aliphatic side chain, or a leucine amino acid residue or a homolog thereof, such as, for example, a residue selected from the group consisting of a leucine amino acid residue, an isoleucine amino acid residue, a homoleucine amino acid residue, an alloisoleucine amino acid residue, a norleucine amino acid residue, and a tert-leucine amino acid residue, wherein the homolog may be a D stereoisomer or an L stereoisomer;
each of X³, X⁵, X⁷, X⁸, X⁹, X¹⁰, X¹¹, X¹², and X¹³ is independently an amino acid residue which may be a D stereoisomer or an L stereoisomer; and
each of X⁹, X¹⁰, X¹¹, X¹², and X¹³ is optionally present.

In some embodiments, X⁵ is Z.

In some embodiments, the present disclosure provides a peptide, wherein the peptide is or comprises:

(SEQ ID NO: 2)
B-Z-X³-J-X⁵-Z-X⁷-X⁸-X⁹-X¹⁰-X¹¹-X¹²-X¹³,

or a salt thereof, wherein R^{a} is or comprises a peptide moiety, and each other variable is independently as described herein. In some embodiments, R^{a} is R′—[X]d-. In some embodiments, R′ is R—C(O)—. In some embodiments, R is optionally substituted C₁₋₁₀ aliphatic. In some embodiments, R is optionally substituted C₁₋₁₀ alkyl. In some embodiments, R is methyl.

In another aspect, the present disclosure provides a peptide, wherein the peptide is or comprises:

B′-Z-X³-J′-X⁵-Z-X⁷-X⁸-X⁹-X¹⁰-X¹¹-X¹²-X¹³,

or a salt thereof, wherein each variable is independently as described herein.

In some embodiments, the present disclosure provides a peptide, wherein the peptide is or comprises:

B′-Z-X³-J′-X⁵-Z-X⁷-X⁸-X⁹-X¹⁰-X¹¹-X¹²-X¹³,

or a salt thereof, wherein R^{a} is or comprises a peptide moiety, and each other variable is independently as described herein. In some embodiments, R^{a} is R′—[X]d-. In some embodiments, R′ is R—C(O)—. In some embodiments, R is optionally substituted C₁₋₁₀ aliphatic. In some embodiments, R is optionally substituted C₁₋₁₀ alkyl. In some embodiments, R is methyl.

In some embodiments, each amino acid residue (e.g., X³, X⁵, X⁷, X⁸, X⁹, X¹⁰, X¹¹, X¹², and X¹³, X, Z, etc.) is independently a natural amino acid residue. In some embodiments, one or more amino acid residues are independently a homolog of a natural amino acid residue.

In various embodiments, J is:

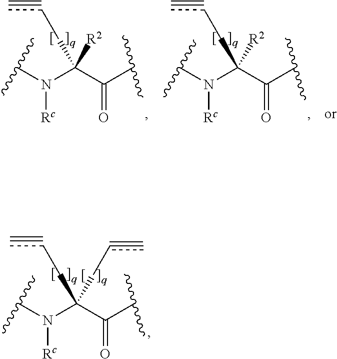

or a salt or a stereoisomeric form thereof, wherein:
each instance of q is independently 1, 2, or 3; and
each instance of [symbol] independently represents a single bond, a double bond or a triple bond.

In some embodiments [symbol] is a single bond. In some embodiments, [symbol] is a double bond. In some embodiments, [symbol] is a triple bond.

In some embodiments, R¹ is —H. In some embodiments, R¹ is optionally substituted C₁₋₆ alkyl. In some embodiments, R¹ is methyl.

In some embodiments, J is S3 residue. In some embodiments, J is a R3 residue. In some embodiments, J is a S4 residue. In some embodiments, J is a R4 residue. In some embodiments, J is a S5 residue. In some embodiments, J is an R5 residue.

In some embodiments, R^{a} is optionally substituted acyl. In some embodiments, R^{a} is —C(O)R, wherein R is as described herein. In some embodiments, R is optionally substituted C₁₋₆ aliphatic. In some embodiments, R is optionally substituted C₁₋₆ alkyl. In some embodiments, R is methyl. In various embodiments, R^{a} is substituted or unsubstituted acetyl. In some embodiments, R^{a} is CH₃C(O)—.

In some embodiments, B¹ is B as described herein. In some embodiments, B¹ is B′ as described herein. In some embodiments, B is

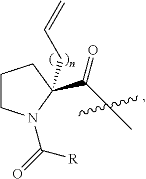

wherein n is 1-10, and R is as described herein. In some embodiments, B is

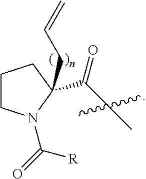

In some embodiments, R is methyl. In some embodiments, B is

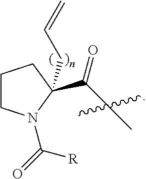

In some embodiments, B is

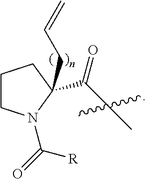

In some embodiments, B′ is

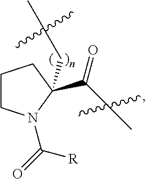

wherein n is 1-10, and R is as described herein. In some embodiments, B′ is

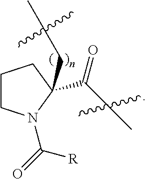

In some embodiments, R is methyl. In some embodiments, B′ is

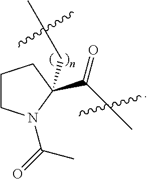

In some embodiments, B′ is

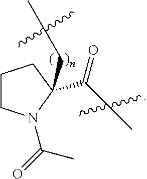

In some embodiments, n is 1, 2, 3, 4, 5, 6, 7, 8, 9, or 10. In some embodiments, n is 1. In some embodiments, n is 2. In some embodiments, n is 3. In some embodiments, n is 4. In some embodiments, n is 5. In some embodiments, B is

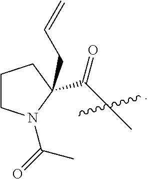 [SMILES: *C(=O)[C@@]1(CC=C)CCCN1C(C)=O]

In some embodiments, B is

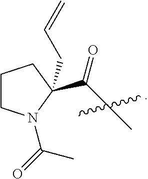 [SMILES: *C(=O)[C@]1(CC=C)CCCN1C(C)=O]

In some embodiments, B′ is

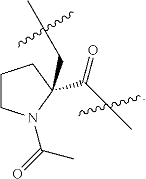 [SMILES: *C[C@]1(C(*)=O)CCCN1C(C)=O]

In some embodiments, B′ is

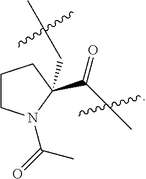 [SMILES: *C[C@@]1(C(*)=O)CCCN1C(C)=O]

In various embodiments, B is selected from the group consisting of:

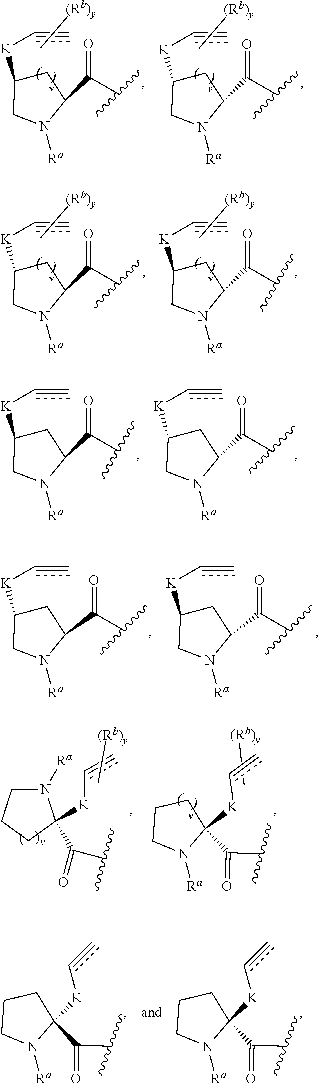

or a salt or a stereoisomeric form thereof. In some embodiments, [symbol] is a single bond. In some embodiments, [symbol] a double bond. In some embodiments, [symbol] is a triple bond. In various embodiments, B′ is selected from the group consisting of

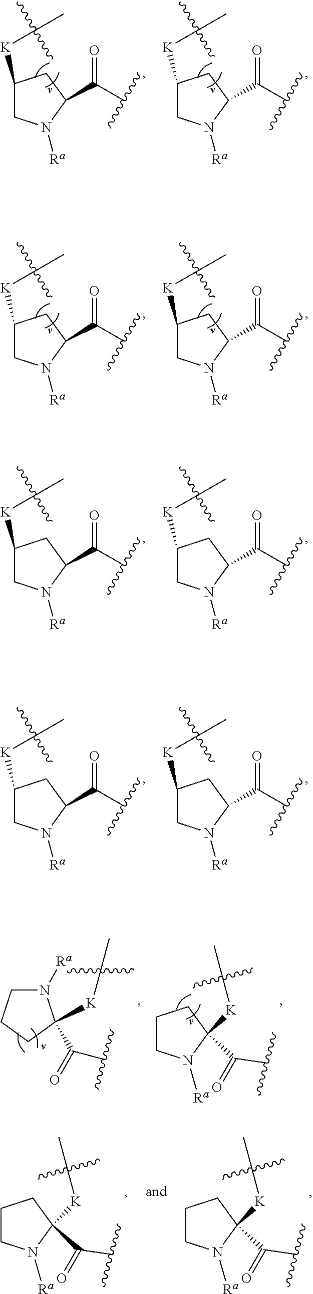

or a salt or a stereoisomeric form thereof. In some embodiments, K is optionally substituted C₁₋₁₀ alkylene. In some embodiments, K is —CH₂—. In some embodiments, K is —CH₂CH₂—. In some embodiments, K is —CH₂CH₂CH₂—. In some embodiments, B is a N-acetyl-PL3 residue.

In some embodiments, v is 1. In some embodiments, v is 2.

In some embodiments, K is bonded to alpha-carbon of B or B′. In some embodiments, K is an optionally substituted bivalent aliphatic. In some embodiments, K is an optionally substituted bivalent heteroaliphatic. In some embodiments, K is optionally substituted C₁₋₁₀ alkylene. In some embodiments, K is —CH₂—. In some embodiments, K is —CH₂CH₂—. In some embodiments, K is —CH₂CH₂CH₂—. In some embodiments, K is —CH₂CH₂CH₂CH₂—. In some embodiments, K is —CH₂CH₂CH₂CH₂CH₂—.

In some embodiments, R^{b} is —H.

In some embodiments, J′ is

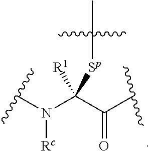

In some embodiments, J′ is

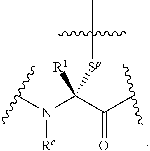

In some embodiments, R¹ is —H. In some embodiments, R¹ is optionally substituted C₁₋₆ alkyl. In some embodiments, R¹ is methyl.

In some embodiments, R^{c} is —H. In some embodiments, R^{c} is optionally substituted C₁₋₆ alkyl. In some embodiments, R^{c} is methyl.

In some embodiments, S^{L} is optionally substituted —CH═CH—. In some embodiments, S^{p1} is —CH═CH—. In some embodiments, S^{p} is —CH═CH—S^{p2}_S^{p3}. In some embodiments, S^{L} is a covalent bond. In some embodiments, S^{p1} is a covalent bond. In some embodiments, S^{p2} is a covalent bond. In some embodiments, S^{p3} is a covalent bond. In some embodiments, S^{L} is optionally substituted C₁₋₆ alkyl. In some embodiments, S^{p2} is optionally substituted C₁₋₆ alkyl. In some embodiments, S^{p3} is optionally substituted C₁₋₆ alkyl. In some embodiments, an optionally substituted C₁₋₆ alkyl is —CH₂—. In some embodiments, it is —(CH₂)₂—. In some embodiments, it is —(CH₂)₃—. In some embodiments, it is —(CH₂)₄—. In some embodiments, it is —(CH₂)₅—. In some embodiments, S^{p} is —CH═CH—CH₂—. In some embodiments, S^{p} is —CH═CH—CH₂CH₂—. In some embodiments, S^{p} is —CH═CH—CH₂CH₂CH₂—. In some embodiments, K is —CH₂—, S^{p} is —CH═CH—CH₂CH₂CH₂—. In some embodiments, —CH═CH— is cis. In some embodiments, —CH═CH— is trans.

In some embodiments, X² is an amino acid residue or a homolog thereof selected from a leucine amino acid residue, an isoleucine amino acid residue, an alanine amino acid residue, a cyclopropyl alanine amino acid residue, a lysine amino acid residue, and a threonine amino acid residue, wherein the homolog may be a D stereoisomer or an L stereoisomer. In some embodiments, X² is a leucine amino acid residue or a homolog thereof, wherein the homolog may be a D stereoisomer or an L stereoisomer. In some embodiments, X² is an alanine amino acid residue or a homolog thereof, wherein the homolog may be a D stereoisomer or an L stereoisomer.

In some embodiments, X³ is an amino acid residue or a homolog thereof selected from a histidine amino acid residue, a norleucine amino acid residue, a leucine amino acid residue, and an arginine amino acid residue, wherein the homolog may be a D stereoisomer or an L stereoisomer. In some embodiments, X³ is a leucine amino acid residue or a homolog thereof, wherein the homolog may be a D stereoisomer or an L stereoisomer.

In some embodiments, X⁵ is an amino acid residue or a homolog thereof selected from an arginine amino acid residue, an asparagine amino acid residue, a leucine amino acid residue, a tyrosine amino acid residue, a norleucine amino acid residue, a cyclopropyl alanine amino acid residue, and a histidine amino acid residue, wherein the homolog may be a D stereoisomer or an L stereoisomer. In some embodiments, X⁵ is an arginine amino acid residue or a homolog thereof, wherein the homolog may be a D stereoisomer or an L stereoisomer. In some embodiments, X⁵ is a tyrosine amino acid residue or a homolog thereof, wherein the homolog may be a D stereoisomer or an L stereoisomer.

In some embodiments, X⁶ is an amino acid residue or a homolog thereof selected from a leucine amino acid residue, a histidine amino acid residue, a tyrosine amino acid residue, and a norleucine amino acid residue, wherein the homolog may be a D stereoisomer or an L stereoisomer. In some embodiments, X⁶ is a leucine amino acid residue or a homolog thereof, wherein the homolog may be a D stereoisomer or an L stereoisomer.

In some embodiments, X⁷ is an amino acid residue or a homolog thereof selected from a leucine amino acid residue, a glutamine amino acid residue, a histidine amino acid residue, and an alanine amino acid residue, wherein the homolog may be a D stereoisomer or an L stereoisomer. In some embodiments, X⁷ is a leucine amino acid residue or a homolog thereof, wherein the homolog may be a D stereoisomer or an L stereoisomer.

In some embodiments, X⁸ is an amino acid residue or a homolog thereof selected from an glutamine amino acid residue, a leucine amino acid residue, a histidine amino acid residue, a threonine amino acid residue, an alanine amino acid residue, a tyrosine amino acid residue, an aspartic acid amino acid residue, and an asparagine amino acid residue, wherein the homolog may be a D stereoisomer or an L stereoisomer. In some embodiments, X⁸ is a glutamine amino acid residue or a homolog thereof, wherein the homolog may be a D stereoisomer or an L stereoisomer. In some embodiments, X⁸ is an aspartic acid amino acid residue or a homolog thereof, wherein the homolog may be a D stereoisomer or an L stereoisomer. In some embodiments, X⁸ is a tyrosine amino acid residue or a homolog thereof, wherein the homolog may be a D stereoisomer or an L stereoisomer.

In some embodiments, X⁹ is an amino acid residue or a homolog thereof selected from a tyrosine amino acid residue, an aspartic acid amino acid residue, and an asparagine amino acid residue, wherein the homolog may be a D stereoisomer or an L stereoisomer. In some embodiments, X⁹ is an aspartic acid amino acid residue, wherein the homolog may be a D stereoisomer or an L stereoisomer. In some embodiments, X⁹ is a tyrosine amino acid residue, wherein the homolog may be a D stereoisomer or an L stereoisomer.

In some embodiments, the present disclosure provides a peptide selected from

| | |
|---|---|
| PL3-S5 unstapled | Ac-PL3-Lys-Leu-S5-Asn-Leu-Leu-Thr-Tyr-NHMe |
| PL3-S5 stapled | Ac-PL3-Lys-Leu-S5-Asn-Leu-Leu-Thr-Tyr-NHMe |
| PL3-S4 unstapled | Ac-PL3-Lys-Leu-S4-Asn-Leu-Leu-Thr-Tyr-NHMe |
| PL3-S4 stapled | Ac-PL3-Lys-Leu-S4-Asn-Leu-Leu-Thr-Tyr-NHMe |
| PL3-S3 unstapled | Ac-PL3-Lys-Leu-S3-Asn-Leu-Leu-Thr-Tyr-NHMe |
| PL3-S3 stapled | Ac-PL3-Lys-Leu-S3-Asn-Leu-Leu-Thr-Tyr-NHMe |
| PL3-R5 unstapled | Ac-PL3-Lys-Leu-R5-Asn-Leu-Leu-Thr-Tyr-NHMe |
| PL3-R5 stapled | Ac-PL3-Lys-Leu-R5-Asn-Leu-Leu-Thr-Tyr-NHMe |
| PL3-R4 unstapled | Ac-PL3-Lys-Leu-R4-Asn-Leu-Leu-Thr-Tyr-NHMe |
| PL3-R4 stapled | Ac-PL3-Lys-Leu-R4-Asn-Leu-Leu-Thr-Tyr-NHMe |
| PL3-R3 unstapled | Ac-PL3-Lys-Leu-R3-Asn-Leu-Leu-Thr-Tyr-NHMe |
| PL3-R3 stapled | Ac-PL3-Lys-Leu-R3-Asn-Leu-Leu-Thr-Tyr-NHMe |
| PLL4-1 | Ac-PL3-Ile-Leu-S5-Arg-Leu-Leu-Gln-Tyr-NHMe |
| PLL4-2 | Ac-PL3-Leu-Leu-S5-Arg-His-Leu-Leu-Tyr-NHMe |
| PLL4-3 | Ac-PL3-Leu-Leu-S5-Arg-Leu-Leu-His-Tyr-NHMe |
| PLL4-4 | Ac-PL3-Ala-Leu-S5-Arg-Tyr-Leu-Leu-Asp-NHMe |
| PLL4-5 | Ac-PL3-Lys-Leu-S5-Asn-Leu-Leu-Thr-Tyr-NHMe |
| PLL4-6 | Ac-PL3-Ala-Leu-S5-Leu-Nle-Leu-Ala-Tyr-NHMe |
| PLL4-7 | Ac-PL3-Leu-His-S5-Leu-Leu-Gln-Tyr-NHMe |
| PLL4-9 | Ac-PL3-Leu-Nle-S5-Leu-Leu-His-Tyr-NHMe |
| PLL4-10 | Ac-PL3-Leu-Leu-S5-Tyr-Leu-Leu-Asp-NHMe |
| PLL4-12 | Ac-PL3-Leu-Arg-S5-Nle-Leu-Ala-Tyr-NHMe |
| PLL5-1 | Ac-PL3-Leu-Leu-S5-Tyr-Leu-Leu-Asp-Tyr-NHMe |
| PLL5-2 | Ac-PL3-Leu-Leu-S5-Tyr-Leu-Leu-Asp-Asp-NHMe |
| PLL5-3 | Ac-PL3-Leu-Leu-S5-Tyr-Leu-Leu-Gln-Tyr-NHMe |
| PLL5-4 | Ac-PL3-Ile-Leu-S5-Tyr-Leu-Leu-Asp-NHMe |
| PLL5-5 | Ac-PL3-Thr-Leu-S5-Tyr-Leu-Leu-Asp-NHMe |
| PLL5-6 | Ac-PL3-Lys-Leu-S5-Tyr-Leu-Leu-Asp-NHMe |
| PLL5-8 | Ac-PL3-Leu-Leu-S5-Arg-Leu-Leu-Asp-NHMe |
| PLL5-10 | Ac-PL3-Leu-Leu-S5-Tyr-Leu-Leu-Thr-NHMe |
| PLL5-11 | Ac-PL3-Leu-Leu-S5-Tyr-Leu-Leu-His-NHMe |
| PLL5-12 | Ac-PL3-Leu-Leu-S5-Tyr-Leu-Leu-Gln-NHMe |
| PLL7-1 | Ac-PL3-Thr-Leu-S5-Tyr-Leu-Leu-Asp-Asp-NHMe |
| PLL7-5 | Ac-PL3-Cpa-Leu-S5-Arg-Leu-Leu-Gln-Asp-NHMe |
| PLL7-7 | Ac-PL3-Cpa-Leu-S5-Tyr-Leu-Leu-Gln-Asp-NHMe |
| PLL7-8 | Ac-PL3-Ala-Leu-S5-Tyr-Leu-Leu-Gln-Asp-NHMe |
| PLL7-9 | Ac-PL3-Leu-Leu-S5-Arg-Leu-Leu-His-Asp-NHMe |
| PLL7-11 | Ac-PL3-Ala-Leu-S5-Cpa-Leu-Leu-Asn-Asn-NHMe |
| PLL7-20 | Ac-PL3-Ala-Leu-S5-His-Leu-Leu-Asn-NHMe |
| PLL7-23 | Ac-PL3-Thr-Leu-S5-Cpa-Leu-Leu-Thr-NHMe |
| PLL4-5 variant-1 | H-PL3-Lys-Leu-S5-Asn-Leu-Leu-Thr-Tyr-NHMe |
| PLL4-5 variant-3 | Ac-PL3-Lys-Leu-S5-Asn-Leu-Leu-Thr-Tyr-NH2 |
| PLL4-5 variant-4 | Ac-S5-Ala-Lys-Leu-S5-Asn-Leu-Leu-Thr-Tyr-NHMe |
| PLL4-5 variant-5 | Ac-PL3-Lys-Leu-S5-Asn-Leu-Leu-Thr-Tyr-NHMe |
| PLL4-10 variant-1 | H-PL3-Leu-Leu-S5-Tyr-Leu-Leu-Asp-NHMe |
| PLL4-10 variant-2 | Ac-PL3-Lec-Leu-S5-Tyr-Leu-Leu-Asp-NHMe |
| PLL4-10 variant-3 | Ac-PL3-Leu-Leu-S5-Tyr-Leu-Leu-Asp-NH2 |
| PLL4-10 variant-4 | Ac-S5-Ala-Leu-Leu-S5-Tyr-Leu-Leu-Asp-NHMe |
| PLL4-10 variant-5 | Ac-PL3-Leu-Leu-S5-Tyr-Leu-Leu-Asp-NHMe |
| PLL5-2 variant-1 | H-PL3-Leu-Leu-S5-Tyr-Leu-Leu-Asp-Asp-NHMe |
| PLL5-2 variant-2 | Ac-PL3-Lec-Leu-S5-Tyr-Leu-Leu-Asp-Asp-NHMe |
| PLL5-2 variant-5 | Ac-PL3-Leu-Leu-S5-Tyr-Leu-Leu-Asp-Asp-NHMe |
| PLL7-7 variant-1 | H-PL3-Cpa-Leu-S5-Tyr-Leu-Leu-Gln-Asp-NHMe |
| PLL7-7 variant-2 | Ac-PL3-Lec-Leu-S5-Tyr-Leu-Leu-Gln-Asp-NHMe |
| PLL7-7 variant-3 | Ac-PL3-Cpa-Leu-S5-Tyr-Leu-Leu-Gln-Asp-NH2 |
| PLL7-7 variant-4 | Ac-S5-Ala-Cpa-Leu-S5-Tyr-Leu-Leu-Gln-Asp-NHMe |
| PLL7-7 variant-5 | Ac-PL3-Cpa-Leu-S5-Tyr-Leu-Leu-Gln-Asp-NHMe |
| PLL7-9 variant-1 | H-PL3-Leu-Leu-S5-Arg-Leu-Leu-His-Asp-NHMe |
| PLL7-9 variant-2 | Ac-PL3-Lec-Leu-S5-Arg-Leu-Leu-His-Asp-NHMe |
| PLL7-9 variant-3 | Ac-PL3-Leu-Leu-S5-Arg-Leu-Leu-His-Asp-NH2 |
| PLL7-9 variant-4 | Ac-S5-Ala-Leu-Leu-S5-Arg-Leu-Leu-His-Asp-NHMe |
| PLL7-9 variant-5 | Ac-PL3-Leu-Leu-S5-Arg-Leu-Leu-His-Asp-NHMe |
| PLL7-20 variant-3 | Ac-PL3-Ala-Leu-S5-His-Leu-Leu-Asn-NH2 |
| PLL7-20 variant-4 | Ac-S5-Ala-Ala-Leu-S5-His-Leu-Leu-Asn-NHMe |
| PLL7-20 variant-5 | Ac-PL3-Ala-Leu-S5-His-Leu-Leu-Asn-NHMe |

As is known in the art, “affinity” is a measure of the tightness with a particular ligand (e.g., an agent) binds to its partner (e.g., the estrogen receptor or a portion thereof such as the ligand-binding domain of the estrogen receptor). In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

Affinity can be measured in different ways. In some embodiments, affinity is represented as a half-maximal effective concentration (EC₅₀) of the indicated stapled peptide for its target. In some embodiments, the EC₅₀ is the concentration of the indicated peptide that gives a half-maximal response. In some embodiments, affinity is represented as the half-maximal inhibitory concentration (IC₅₀) of the indicated stapled peptide in inhibiting its target. In some embodiments, the IC₅₀ is the concentration of the indicated peptide where the binding to its target is reduced by half. For both EC₅₀ and IC₅₀, the lower the number, the higher the affinity. In some embodiments, a high affinity is achieved with an IC₅₀ that is less than 1.3 uM, or less than 1.0 uM, or less than 0.75 uM (750 nM), or less than 0.5 uM (500 nM), or less than 0.25 uM (250 uM), or less than 0.1 uM (100 nM), or less than 75 nM or less than 50 nM, or less than 25 nM, or less than 10 nM, or less than 5 nM. In some embodiments, a high affinity is achieved with an EC₅₀ that is less than 1.3 uM, or less than 1.0 uM, or less than 0.75 uM (750 nM), or less than 0.5 uM (500 nM), or less than 0.25 uM (250 uM), or less than 0.1 uM (100 nM), or less than 75 nM or less than 50 nM, or less than 25 nM, or less than 10 nM, or less than 5 nM.

In various embodiments, a peptide described herein binds to its target with high affinity.

In some embodiments, the peptide binds to the estrogen receptor with high affinity. In some embodiments, a peptide binds to an estrogen receptor with a half maximal effective concentration (EC50) of less than about 3.0 uM. In some embodiments, the peptide binds to the estrogen receptor with an EC50 of less than about 1.0 uM.

Based the discovery, as described herein, that N-terminal amide proton cloaking of helices via the introduction of an N-terminal proline cap that is conformationally stabilized by a hydrocarbon stapling system results in a peptide that is able to penetrate a cell membrane (as assessed, for example, by the PAMPA assay) as well as have high affinity for a target (e.g., an intracellular target such as the estrogen receptor ligand binding domain), the addition of a second staple to hold the C-terminal portion of the peptide in helical formation is predicted to result in additional improvements in the cell membrane penetration properties and/or affinity of the peptide.

Accordingly, in some embodiments, the peptides described herein further comprise at least one additional staple, where the additional staple is not at the N-terminus of the peptide. Staples for peptides are known and described in, for example, PCT Publication Nos. WO2014/159969 and WO2019/051327, and in U.S. Pat. No. 10,487,110.

In some embodiments, the peptide comprises an amino acid residue analog that is attached to two staples.

Accordingly, in some embodiments of the peptides described herein, J is:

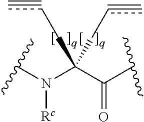

or a salt or stereoisomeric form thereof, and
X⁸ is.

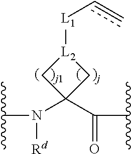

or a salt or stereoisomeric form thereof; wherein:
R^{d} is hydrogen; acyl; substituted or unsubstituted C₁₋₆ alkyl; or an amino protecting group;
L₁ is independently, a bond, a substituted or unsubstituted bivalent C₁₋₁₀ aliphatic or heteroaliphatic, a substituted or unsubstituted C₁₋₁₀ alkylene, —C(O)O—, or —C(═O)R³—;
L₂ is independently a bond, N, optionally substituted CH, or C(R⁴);
R⁵ is, independently, hydrogen; acyl; substituted or unsubstituted C₁₋₆ alkyl; or an amino protecting group;
each of R³ and R⁴ is independently hydrogen, halogen, —NO₂, —OH, —CN, or C₁₋₆ alkyl;
each of j and j1 is independently 0, 1, 2, 3, 4, 5, 6, 7, 8, 9, or 10; and
each instance of [symbol] independently represents a single bond, a double bond or a triple bond.

In some embodiments, J is:

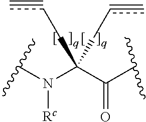

or a salt or stereoisomeric form thereof;
each of X⁹, X¹⁰, and X¹¹ is present; and
X¹¹ is:

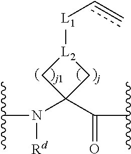

or a salt or stereoisomeric form thereof; wherein:
R^{d} is hydrogen; acyl; substituted or unsubstituted C₁₋₆ alkyl; or an amino protecting group; each instance of [symbol] independently represents a single bond, a double bond or a triple bond;
L₁ is independently, a bond, a substituted or unsubstituted bivalent C₁₋₁₀ aliphatic or heteroaliphatic, a substituted or unsubstituted C₁₋₁₀ alkylene, —C(O)O—, or —C(═O)OR³—;
L₂ is independently a bond, N, optionally substituted CH, or C(R⁴);
R⁵ is, independently, hydrogen; acyl; substituted or unsubstituted C₁₋₆ alkyl; or an amino protecting group;
each of R³ and R⁴ is independently hydrogen, halogen, —NO₂, —OH, —CN, or C₁₋₆ alkyl; and
each of j and j1 is independently 0, 1, 2, 3, 4, 5, 6, 7, 8, 9, or 10; and
each instance of [symbol] independently represents a single bond, a double bond or a triple bond.

In another aspect, the present disclosure provides a peptide, wherein the peptide is or comprises:

B-X²-X³-J-X⁵-X⁶-X⁷-O-X⁹-X¹⁰-X¹¹-X¹²-X¹³

or a salt thereof, wherein:
B is

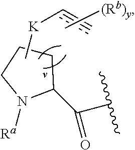

or a salt or a stereoisomeric form thereof, wherein:
v is 1 or 2;
K is a covalent bond, or an substituted or unsubstituted bivalent group selected from a bivalent aliphatic group, alkylene, alkenylene, alkynylene, a bivalent heteroaliphatic group, heteroalkylene, heteroalkenylene, heteroalkynylene, heterocyclene, carbocyclene, arylene, and heteroarylene;
R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; or substituted or unsubstituted heteroarylene;
each instance of R^{b}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
y is 0, 1, 2, or 3; and
each instance of [symbol] independently represents a single bond, a double bond or a triple bond;

J is

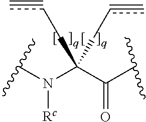

or a salt or a stereoisomeric form thereof, wherein:
each instance of q is independently 1, 2, or 3;
each instance of [symbol] independently represents a single bond, a double bond or a triple bond;
R^{c}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
and
O is of formula

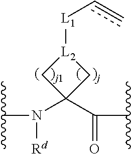

or a salt or stereoisomeric form thereof, wherein:
R^{d} is hydrogen; acyl; substituted or unsubstituted C₁₋₆ alkyl; or an amino protecting group;
each instance of [symbol] independently represents a single bond, a double bond or a triple bond;
L₁ is independently, a bond, a substituted or unsubstituted bivalent C₁₋₁₀ aliphatic or heteroaliphatic, a substituted or unsubstituted C₁₋₁₀ alkylene, —C(O)O—, or —C(═O)OR³—;
L₂ is independently a bond, N, optionally substituted CH, or C(R⁴);
R⁵ is, independently, hydrogen; acyl; substituted or unsubstituted C₁₋₆ alkyl; or an amino protecting group;
each of R³ and R⁴ is independently hydrogen, halogen, —NO₂, —OH, —CN, or C₁₋₆ alkyl;
each of j and j1 is independently 0, 1, 2, 3, 4, 5, 6, 7, 8, 9, or 10; and
each of X², X³, X⁵, X⁶, X⁷, X⁹, X¹⁰, X¹¹, X¹², and X¹³ is independently an amino acid residue which may be a D stereoisomer or an L stereoisomer; and
   each of X⁹, X¹⁰, X¹¹, X¹², and X¹³ is optionally present.

In some embodiments, the present disclosure provides a peptide, wherein the peptide is or comprises:

B-X²-X³-J-X⁵-X⁶-X⁷-O-X⁹-x¹⁰-X¹¹-x¹²-X¹³

or a salt thereof, wherein R^{a} is or comprises a peptide moiety, and each other variable is independently as described herein. In some embodiments, R^{a} is R′—[X]d-. In some embodiments, R′ is R—C(O)—. In some embodiments, R is optionally substituted C₁₋₁₀ aliphatic. In some embodiments, R is optionally substituted C₁₋₁₀ alkyl. In some embodiments, R is methyl.

In another aspect, the present disclosure provides a peptide, wherein the peptide is or comprises:

B′-X²-X³-J″-X⁵-X⁶-X⁷-O′-X⁹-X¹⁰-X¹¹-X¹²-X¹³

or a salt thereof, wherein:
J″ is

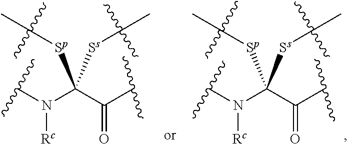

or a salt or a stereoisomeric form thereof, wherein:
S^{p} is —S^{p1}—S^{p2}—S^{p3}—, wherein S^{p1} is bonded to K;
S^{s} is —S^{s1}—S^{s2}—S^{s3}—, wherein S^{s3} is bonded to L₁;
O′ is of formula:

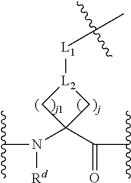

or a salt or a stereoisomeric form thereof;
each of S^{p1}, S^{p2}, S^{p3}, S^{s1}, S^{s2}, and S^{s3} is independently S^{L}; and wherein each other variable is independently as described herein.

In some embodiments, the present disclosure provides a peptide, wherein the peptide is or comprises:

B′-X²-X³-J″-X⁵-X⁶-X⁷-O′-X⁹-X¹⁰-X¹¹-x¹²-X¹³

or a salt thereof, wherein R^{a} is or comprises a peptide moiety, and each other variable is independently as described herein. In some embodiments, R^{a} is R′—[X]d-. In some embodiments, R′ is R—C(O)—. In some embodiments, R is optionally substituted C₁₋₁₀ aliphatic. In some embodiments, R is optionally substituted C₁₋₁₀ alkyl. In some embodiments, R is methyl.

In another aspect, the present disclosure provides a peptide, wherein the peptide is or comprises:

B-X²-X³-J-X⁵-X⁶-X⁷-X⁸-X⁹-X¹⁰-O-X¹²-X¹³-X¹⁴,

or a salt form thereof, wherein:
each of X², X³, X⁵, X⁶, X⁸, X⁹, X¹⁰, X¹², X¹³, and X¹⁴ is independently an amino acid residue which may be a D stereoisomer or an L stereoisomer;
each of X⁸, X⁹, X¹⁰, X¹², X¹³, and X¹⁴ is optionally present;
In some embodiments, the present disclosure provides a peptide, wherein the peptide is or comprises:

B-X²-X³-J-X⁵-X⁶-X⁷-X⁸-X⁹-X¹⁰-O-X¹²-X¹³-X¹⁴,

or a salt thereof, wherein R^{a} is or comprises a peptide moiety, and each other variable is independently as described herein. In some embodiments, R^{a} is R′—[X]d-. In some embodiments, R′ is R—C(O)—. In some embodiments, R is optionally substituted C₁₋₁₀ aliphatic. In some embodiments, R is optionally substituted C₁₋₁₀ alkyl. In some embodiments, R is methyl.

In some embodiments, the present disclosure provides a peptide, wherein the peptide is or comprises:

B′-X²-X³-J″-X⁵-X⁶-X⁷-X⁸-X⁹-X¹⁰-O-X¹²-X¹³-X¹⁴

or a salt form thereof, wherein each variable is independently as described herein.

In some embodiments, the present disclosure provides a peptide, wherein the peptide is or comprises:

B′-X²-X³-J″-X⁵-X⁶-X⁷-X⁸-X⁹-X¹⁰-O-X¹²-X¹³-X¹⁴

or a salt thereof, wherein R^{a} is or comprises a peptide moiety, and each other variable is independently as described herein. In some embodiments, R^{a} is R′—[X]d-. In some embodiments, R′ is R—C(O)—. In some embodiments, R is optionally substituted C₁₋₁₀ aliphatic. In some embodiments, R is optionally substituted C₁₋₁₀ alkyl. In some embodiments, R is methyl.

In some embodiments, S^{L} is optionally substituted —CH═CH—. In some embodiments, S^{s2} is —CH═CH—. In some embodiments, S^{s} is —S^{p1}—CH═CH—S^{p3}—. In some embodiments, S^{L} is a covalent bond. In some embodiments, S^{s1} is a covalent bond. In some embodiments, S^{s2} is a covalent bond. In some embodiments, S^{s3} is a covalent bond. In some embodiments, S^{L} is optionally substituted C₁₋₆ alkyl. In some embodiments, S^{s1} is optionally substituted C₁₋₆ alkyl. In some embodiments, S^{s3} is optionally substituted C₁₋₆ alkyl. In some embodiments, an optionally substituted C₁₋₆ alkyl is —CH₂—. In some embodiments, it is —(CH₂)₂—. In some embodiments, it is —(CH₂)₃—. In some embodiments, it is —(CH₂)₄—. In some embodiments, it is —(CH₂)₅—. In some embodiments, S^{p} is —(CH₂)ₘ—CH═CH—(CH₂)ₙ—, wherein each m and n is independently 1-10. In some embodiments, —CH═CH— is cis. In some embodiments, —CH═CH— is trans.

In various embodiments of the peptides described herein, B is selected from:

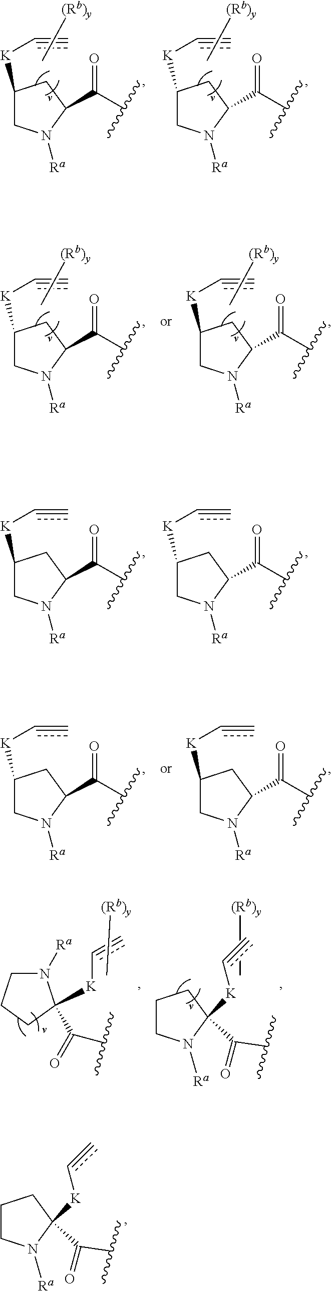

or a salt or stereoisomeric form thereof

In yet another aspect, the present disclosure provides a peptide having the structure of:

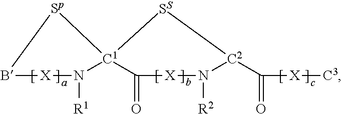

or a salt or a stereoisomer thereof, wherein:
B′ is

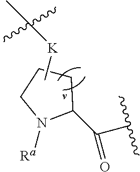

or a salt or stereoisomeric form thereof; wherein:
v is 1 or 2;
K is a hydrogen; a substituted or unsubstituted aliphatic; a substituted or unsubstituted alkylene; a substituted or unsubstituted alkynylene; a substituted or unsubstituted heteroaliphatic; a substituted or unsubstituted aryl; a substituted or unsubstituted heteroaryl; a substituted or unsubstituted acyl; a substituted or unsubstituted hydroxyl; a substituted or unsubstituted thiol; a substituted or unsubstituted amino; or a halo;
R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; or substituted or unsubstituted heteroarylene;
each of R¹ and R² is independently R′;
C³ is R′, —OR′ or —N(R′)₂;
each of X is independently an amino acid residue which may be a D stereoisomer or an L stereoisomer;
each of a, b, and c is independently 1, 2, 3, 4, 5, 6, 7, 8, 9 10, 11, 12, 13, 14, 15, 16, 17, 18, 19, or 20;
C¹ is a carbon atom;
C² is of the formula:

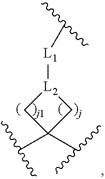

or a salt or stereoisomeric form thereof, wherein:
L₁ is independently, a bond, a substituted or unsubstituted bivalent C₁₋₁₀ aliphatic or heteroaliphatic, a substituted or unsubstituted C₁₋₁₀ alkylene, —C(O)O—, or —C(═O)OR³—;
L₂ is independently a bond, N, optionally substituted CH, or C(R⁴);
R⁵ is, independently, hydrogen; acyl; substituted or unsubstituted C₁₋₆ alkyl; or an amino protecting group;
each of R³ and R⁴ is independently hydrogen, halogen, —NO₂, —OH, —CN, or C₁₋₆ alkyl;
each of j and j1 is independently 0, 1, 2, 3, 4, 5, 6, 7, 8, 9, or 10;
S^{p} is —S^{p1}—S^{p2}—S^{p3}—, wherein S^{p1} is bonded to K and S^{p3} is bonded to C¹;
S^{s} is —S^{s1}—S^{s2}—S^{s3}—, wherein S^{s1} is bonded to C¹ and S^{s3} is bonded to L₁;
each of S^{p1}, S^{p2}, S^{p3}, S^{s1}, S^{s2}, and S^{s3} is independently S^{L};
each S^{L} is independently a bond, a substituted or unsubstituted C₁₋₁₀ alkane, a substituted or unsubstituted C₁₋₁₀ alkylene, or an optionally substituted, bivalent C₁-C₂₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

In various embodiments, b is 6, or b is 3, or a is 2. In some embodiments, a is 2. In some embodiments, b is 3. In some embodiments, b is 6. In some embodiments, a is 2 and b is 3. In some embodiments, a is 2 and b is 6.

As described herein, in various embodiments, B′ is

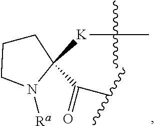

or a salt or a stereoisomeric form thereof. In some embodiments, K is —CH₂—. In some embodiments, R^{a} is optionally substituted acyl. In some embodiments, R^{a} is —C(O)R. In some embodiments, R is optionally substituted C₁₋₆ aliphatic. In some embodiments, R is optionally substituted C₁₋₆ alkyl. In some embodiments, R is methyl.

In some embodiments, Z is an amino acid residue which comprises an optionally substituted C₄₋₆ (e.g., C₄, C₅, or C₆) aliphatic side chain. In some embodiments, Z is an amino acid residue which comprises an optionally substituted C₄₋₆ (e.g., C₄, C₅, or C₆) alkyl side chain. In some embodiments, Z is an amino acid residue which comprises an C₄₋₆ (e.g., C₄, C₅, or C₆) alkyl side chain. In some embodiments, Z is a leucine amino acid residue or a homolog thereof, such as, for example, a residue selected from the group consisting of a leucine amino acid residue, an isoleucine amino acid residue, a homoleucine amino acid residue, an alloisoleucine amino acid residue, a norleucine amino acid residue, and a tert-leucine amino acid residue, wherein the homolog may be a D stereoisomer or an L stereoisomer. In some embodiments, Z is a leucine residue.

In some embodiments, the peptide can form a helix structure.

In some embodiments, the peptide can traverse a phospholipid-infused membrane (e.g., a membrane in a PAMPA assay or a cell membrane).

The present disclosure further provides a method of altering a biological pathway in a cell comprising treating the cell with one or more of the various non-limiting stapled peptides described herein, or salt thereof. Such a method comprises in vitro or in vivo methods (e.g., the cell may be in a subject, such as a cancer cell in a human subject). Such a peptide may be useful as a research tool, e.g., for cellular assays.

The present disclosure provides pharmaceutical compositions comprising one or more of the stapled peptides described herein, or a salt thereof, and a pharmaceutically acceptable excipient. Pharmaceutical compositions comprise compositions for therapeutic use as well as cosmetic compositions. Such compositions may optionally comprise one or more additional therapeutically active agents. In accordance with some embodiments, a method of administering a pharmaceutical composition comprising an inventive composition to a subject in need thereof is provided. In some embodiments, the inventive composition is administered to humans. For the purposes of the present disclosure, the “active ingredient” generally refers to one or more of the various stapled peptides described herein.

Although the descriptions of pharmaceutical compositions provided herein are principally directed to pharmaceutical compositions for administration to humans, it will be understood by the skilled artisan that such compositions are generally suitable for administration to animals of all sorts. Modification of pharmaceutical compositions for administration to various animals is well understood, and the ordinarily skilled veterinary pharmacologist can design and/or perform such modification with merely ordinary, if any, experimentation.

Pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, shaping and/or packaging the product into a desired single- or multi-dose unit.

A pharmaceutical composition of the present disclosure may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

The relative amounts of the active ingredient, the pharmaceutically acceptable excipient, and/or any additional ingredients in a pharmaceutical composition of the present disclosure will vary, depending upon the identity, size, and/or disorder of the subject treated and further depending upon the route by which the composition is to be administered. By way of example, the composition may comprise between 0.1% and 100% (w/w) active ingredient.

As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, a pharmaceutically acceptable excipient includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro, (Lippincott, Williams & Wilkins, Baltimore, M D, 2006) discloses various excipients used in formulating pharmaceutical compositions and known techniques for the preparation thereof. Except insofar as any conventional carrier medium is incompatible with a substance or its derivatives, such as by producing any undesirable biological effect or otherwise interacting in a deleterious manner with any other component(s) of the pharmaceutical composition, its use is contemplated to be within the scope of the present disclosure.

In some embodiments, the pharmaceutically acceptable excipient is at least 95%, 96%, 97%, 98%, 99%, or 100% pure. In some embodiments, the excipient is approved for use in humans and for veterinary use. In some embodiments, the excipient is approved by the United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known. For example, S. M. Berge, et al. describes pharmaceutically acceptable salts in detail in J. Pharmaceutical Sciences, 66: 1-19 (1977). In some embodiments, pharmaceutically acceptable salts include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other known methods such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, pharmaceutically acceptable salts include, but are not limited to, nontoxic base addition salts, such as those formed by acidic groups of provided compounds (e.g., phosphate linkage groups of oligonucleotides, phosphorothioate linkage groups of oligonucleotides, etc.) with bases. Representative alkali or alkaline earth metal salts include salts of sodium, lithium, potassium, calcium, magnesium, and the like. In some embodiments, pharmaceutically acceptable salts are ammonium salts (e.g., —N(R)₃⁺). In some embodiments, pharmaceutically acceptable salts are sodium salts. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the Formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and combinations thereof

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol,); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; and combinations thereof.

Exemplary preservatives may include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate. Exemplary antimicrobial preservatives include, but are not limited to, benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal. Exemplary antifungal preservatives include, but are not limited to, butyl paraben, methyl paraben, ethyl paraben, propyl paraben, benzoic acid, hydroxybenzoic acid, potassium benzoate, potassium sorbate, sodium benzoate, sodium propionate, and sorbic acid. Exemplary alcohol preservatives include, but are not limited to, ethanol, polyethylene glycol, phenol, phenolic compounds, bisphenol, chlorobutanol, hydroxybenzoate, and phenylethyl alcohol. Exemplary acidic preservatives include, but are not limited to, vitamin A, vitamin C, vitamin E, beta-carotene, citric acid, acetic acid, dehydroacetic acid, ascorbic acid, sorbic acid, and phytic acid. Other preservatives include, but are not limited to, tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and combinations thereof

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and combinations thereof.

Exemplary oils include, but are not limited to, almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Liquid dosage forms for oral and parenteral administration include, but are not limited to, pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the present disclosure are mixed with solubilizing agents such as Cremophor, alcohols, oils, modified oils, glycols, polysorbates, cyclodextrins, polymers, and combinations thereof.

Injectable preparations, for example, sterile injectable aqueous or oleaginous suspensions may be formulated according to the known art using dispersing or wetting agents and suspending agents. The sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are typically suppositories which can be prepared by mixing the conjugates of this present disclosure with non-irritating excipients or carriers such as cocoa butter, polyethylene glycol or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Solid compositions of a similar type may be employed as fillers in soft and hard-filled gelatin capsules using such excipients as lactose or milk sugar as well as high molecular weight polyethylene glycols and the like. The solid dosage forms of tablets, capsules, pills, and granules can be prepared with coatings and shells such as enteric coatings and other coatings well known in the pharmaceutical formulating art. They may optionally comprise opacifying agents and can be of a composition that they release the active ingredient(s) only, or preferentially, in a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

The active ingredients (e.g., the peptides described herein) can be in micro-encapsulated form with one or more excipients as noted above. The solid dosage forms of tablets, capsules, pills, and granules can be prepared with coatings and shells such as enteric coatings, release controlling coatings and other coatings well known in the pharmaceutical formulating art. In such solid dosage forms the active ingredient may be admixed with at least one inert diluent such as sucrose, lactose or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner, Examples of embedding compositions which can be used include polymeric substances and waxes.

Dosage forms for topical and/or transdermal administration of a conjugate of this disclosure may include ointments, pastes, creams, lotions, gels, powders, solutions, sprays, inhalants and/or patches. Generally, the active component is admixed under sterile disorders with a pharmaceutically acceptable carrier and/or any needed preservatives and/or buffers as may be required. Additionally, the present disclosure contemplates the use of transdermal patches, which often have the added advantage of providing controlled delivery of an active ingredient to the body. Such dosage forms may be prepared, for example, by dissolving and/or dispensing the active ingredient in the proper medium. Alternatively or additionally, the rate may be controlled by either providing a rate controlling membrane and/or by dispersing the active ingredient in a polymer matrix and/or gel.

Suitable devices for use in delivering intradermal pharmaceutical compositions described herein include short needle devices such as those described in U.S. Pat. Nos. 4,886,499; 5,190,521; 5,328,483; 5,527,288; 4,270,537; 5,015,235; 5,141,496; and 5,417,662, Intradermal compositions may be administered by devices which limit the effective penetration length of a needle into the skin, such as those described in PCT publication WO 99/34850 and functional equivalents thereof. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Alternatively or additionally, conventional syringes may be used in the classical mantoux method of intradermal administration.

Formulations for topical administration include, but are not limited to, liquid and/or semi liquid preparations such as liniments, lotions, oil in water and/or water in oil emulsions such as creams, ointments and/or pastes, and/or solutions and/or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

A pharmaceutical composition of the present disclosure may be prepared, packaged, and/or sold in a formulation for pulmonary administration via the buccal cavity. Such a formulation may comprise dry particles which comprise the active ingredient and which have a diameter in the range from about 0.5 to about 7 nanometers or from about 1 to about 6 nanometers. Such compositions are conveniently in the form of dry powders for administration using a device comprising a dry powder reservoir to which a stream of propellant may be directed to disperse the powder and/or using a self propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions of the present disclosure formulated for pulmonary delivery may provide the active ingredient in the form of droplets of a solution and/or suspension. Such formulations may be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

The formulations described herein as being useful for pulmonary delivery are useful for intranasal delivery of a pharmaceutical composition of the present disclosure. Another formulation for intranasal administration is a coarse powder comprising the active ingredient and having an average particle from about 0.2 to 500 micrometers. Such a formulation is administered in the manner in which snuff is taken, i.e. by rapid inhalation through the nasal passage from a container of the powder held close to the nares.

Formulations for nasal administration may, for example, comprise from about as little as 0.1% (w/w) and as much as 100% (w/w) of the active ingredient, and may comprise one or more of the additional ingredients described herein. A pharmaceutical composition of the present disclosure may be prepared, packaged, and/or sold in a formulation for buccal administration. Such formulations may, for example, be in the form of tablets and/or lozenges made using conventional methods, and may, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition of the present disclosure may be prepared, packaged, and/or sold in a formulation for ophthalmic administration. Such formulations may, for example, be in the form of eye drops including, for example, a 0.1/1.0% (w/w) solution and/or suspension of the active ingredient in an aqueous or oily liquid carrier. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are contemplated as being within the scope of the present disclosure.

General considerations in the formulation and/or manufacture of pharmaceutical agents may be found, for example, in Remington: The Science and Practice of Pharmacy 21^{st} ed., Lippincott Williams & Wilkins, 2005.

Inventive peptides provided herein are typically formulated in dosage unit form for ease of administration and uniformity of dosage. In some embodiments, the peptide (or pharmaceutically acceptable composition comprising the peptide) is administered to a subject (e.g., a human) in a therapeutically effective amount. As used herein, and unless otherwise specified, a “therapeutically effective amount” of a compound (e.g., a stapled peptide as described herein) is an amount sufficient to provide a therapeutic benefit in the treatment of the disease (or disorder) or to delay or minimize one or more symptoms associated with the disease (or disorder) in the treated subject. The term “therapeutically effective amount” can encompass an amount that improves overall therapy, reduces or avoids symptoms or causes of the disease (or disorder), or enhances the therapeutic efficacy of another therapeutic agent.

It will be understood, however, that the total daily usage of the compositions of the present disclosure will be decided by the attending physician within the scope of sound medical judgment. The specific therapeutically effective amount (e.g., the therapeutically effective dose level) for any particular subject will depend upon a variety of factors including the disease (e.g., a disease involving the estrogen receptor, such as breast, ovarian, colorectal prostate, or endometrial cancer), disorder, or disorder being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

One or more of the various stapled peptides described herein, salt thereof, or pharmaceutical composition thereof, may be administered by any route. In some embodiments, the one or more stapled peptides described herein, salt thereof, or pharmaceutical composition thereof, are administered by a variety of routes, including oral, intravenous, intramuscular, intra-arterial, intramedullary, intrathecal, subcutaneous, intraventricular, transdermal, intradermal, rectal, intravaginal, intraperitoneal, topical (as by powders, ointments, creams, and/or drops), mucosal, nasal, bucal, enteral, sublingual; by intratracheal instillation, bronchial instillation, and/or inhalation; and/or as an oral spray, nasal spray, and/or aerosol. Specifically contemplated routes are systemic intravenous injection, regional administration via blood and/or lymph supply, and/or direct administration to an affected site. In general the most appropriate route of administration will depend upon a variety of factors including the nature of the agent (e.g., its stability in the environment of the gastrointestinal tract), and the disorder of the subject (e.g., whether the subject is able to tolerate oral administration). At present the oral and/or nasal spray and/or aerosol route is most commonly used to deliver therapeutic agents directly to the lungs and/or respiratory system. However, the present disclosure encompasses the delivery of the inventive pharmaceutical composition by any appropriate route taking into consideration likely advances in the sciences of drug delivery.

In certain embodiments, a stapled peptide as described herein, or a salt thereof, or pharmaceutical composition thereof, may be administered at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic effect. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. The exact amount of an inventive polypeptide required to achieve an effective amount will vary from subject to subject, depending, for example, on species, age, and general disorder of a subject, severity of the side effects or disorder, identity of the particular compound(s), mode of administration, and the like.

In some embodiments, the present disclosure encompasses “therapeutic cocktails” comprising inventive polypeptides. In some embodiments, the inventive polypeptide comprises a single species which can bind to multiple targets. In some embodiments, different inventive polypeptides comprise different targeting moiety species, and all of the different targeting moiety species can bind to the same target. In some embodiments, different inventive polypeptides comprise different targeting moiety species, and all of the different targeting moiety species can bind to different targets. In some embodiments, such different targets may be associated with the same cell type. In some embodiments, such different targets may be associated with different cell types.

It will be appreciated that inventive polypeptides and pharmaceutical compositions of the present disclosure can be employed in combination therapies. The particular combination of therapies (therapeutics or procedures) to employ in a combination regimen will take into account compatibility of the desired therapeutics and/or procedures and the desired therapeutic effect to be achieved. It will be appreciated that the therapies employed may achieve a desired effect for the same purpose (for example, an inventive conjugate useful for detecting tumors may be administered concurrently with another agent useful for detecting tumors), or they may achieve different effects (e.g., control of any adverse effects).

Pharmaceutical compositions of the present disclosure may be administered either alone or in combination with one or more therapeutically active agents. By “in combination with,” it is not intended to imply that the agents must be administered at the same time and/or formulated for delivery together, although these methods of delivery are within the scope of the present disclosure. The compositions can be administered concurrently with, prior to, or subsequent to, one or more other desired therapeutics or medical procedures. In general, each agent will be administered at a dose and/or on a time schedule determined for that agent. Additionally, the present disclosure encompasses the delivery of the inventive pharmaceutical compositions in combination with agents that may improve their bioavailability, reduce and/or modify their metabolism, inhibit their excretion, and/or modify their distribution within the body. It will further be appreciated that therapeutically active agent and the inventive polypeptides utilized in this combination may be administered together in a single composition or administered separately in different compositions.

The particular combination employed in a combination regimen will take into account compatibility of the therapeutically active agent and/or procedures with the inventive polypeptide and/or the desired therapeutic effect to be achieved. It will be appreciated that the combination employed may achieve a desired effect for the same disorder (for example, an inventive polypeptide may be administered concurrently with another therapeutically active agent used to treat the same disorder), and/or they may achieve different effects (e.g., control of any adverse effects). As used herein, a “therapeutically active agent” refers to any substance used as a medicine for treatment, prevention, delay, reduction or amelioration of a disorder, and refers to a substance that is useful for therapy, including prophylactic and therapeutic treatment. A therapeutically active agent also includes a compound that increases the effect or effectiveness of another compound, for example, by enhancing potency or reducing adverse effects of the various stapled peptides described herein.

In certain embodiments, a therapeutically active agent is an anti-cancer agent, antibiotic, anti-viral agent, anti-HIV agent, anti-parasite agent, anti-protozoal agent, anesthetic, anticoagulant, inhibitor of an enzyme, steroidal agent, steroidal or non-steroidal anti-inflammatory agent, antihistamine, immunosuppressant agent, anti-neoplastic agent, antigen, vaccine, antibody, decongestant, sedative, opioid, analgesic, anti-pyretic, birth control agent, hormone, prostaglandin, progestational agent, anti-glaucoma agent, ophthalmic agent, anti-cholinergic, analgesic, anti-depressant, anti-psychotic, neurotoxin, hypnotic, tranquilizer, anti-convulsant, muscle relaxant, anti-Parkinson agent, anti-spasmodic, muscle contractant, channel blocker, miotic agent, anti-secretory agent, anti-thrombotic agent, anticoagulant, anti-cholinergic, β-adrenergic blocking agent, diuretic, cardiovascular active agent, vasoactive agent, vasodilating agent, anti-hypertensive agent, angiogenic agent, modulators of cell-extracellular matrix interactions (e.g. cell growth inhibitors and anti-adhesion molecules), or inhibitors/intercalators of DNA, RNA, protein-protein interactions, protein-receptor interactions.

In some embodiments, inventive pharmaceutical compositions may be administered in combination with any therapeutically active agent or procedure (e.g., surgery, radiation therapy) that is useful to treat, alleviate, ameliorate, relieve, delay onset of, inhibit progression of, reduce severity of, and/or reduce incidence of one or more symptoms or features of cancer.

A. Kits

The present disclosure also provides a variety of kits comprising one or more of the polypeptides of the present disclosure. For example, the present disclosure provides a kit comprising an inventive polypeptide and instructions for use. A kit may comprise multiple different polypeptides. A kit may comprise any of a number of additional components or reagents in any combination. All of the various combinations are not set forth explicitly but each combination is included in the scope of the present disclosure.

According to certain embodiments of the present disclosure, a kit may include, for example, (I) one or more inventive polypeptides and, optionally, one or more particular therapeutically active agents to be delivered; (ii) instructions for administration to a subject in need thereof.

Kits typically include instructions which may, for example, comprise protocols and/or describe disorders for production of inventive polypeptides, administration of inventive polypeptides to a subject in need thereof, design of novel inventive polypeptide. Kits will generally include one or more vessels or containers so that some or all of the individual components and reagents may be separately housed. Kits may also include a means for enclosing individual containers in relatively close confinement for commercial sale, e.g., a plastic box, in which instructions, packaging materials such as styrofoam, may be enclosed. An identifier, e.g., a bar code, radio frequency identification (ID) tag, may be present in or on the kit or in or one or more of the vessels or containers included in the kit. An identifier can be used, e.g., to uniquely identify the kit for purposes of quality control, inventory control, tracking, movement between workstations.

In order that the present disclosure described herein may be more fully understood, the following examples are set forth. It should be understood that these examples are for illustrative purposes only and are not to be construed as limiting present disclosure in any manner.

### EXAMPLES

In order that the present disclosure described herein may be more fully understood, the following examples are set forth. It should be understood that these examples are for illustrative purposes only and are not to be construed as limiting present disclosure in any manner.

### Example 1

This example describes the development of a ProLock™ staple system and its use in ProLock™ stapled peptides that target various targets, e.g., an estrogen receptor.

In some embodiments, provided peptides comprise amino acid residues whose amino group is a secondary amine, e.g., proline. It has been reported that among the common natural amino acids, proline is the strongest α-helix-initiating residue in proteins (see Yun et al., Proteins: Structure, Function, and Bioinformatics 1991, 10 (3), 219-228; Richardson and Richardson, Science 1988, 240 (4859), 1648). Since proline is unique among natural amino acids in possessing a secondary rather than primary amine, it uniquely removes one N-terminal proton by N-alkylation; in addition, N-acetylation of proline results in formation of an intrahelical hydrogen bond that cloaks one or two of the exposed N-terminal protons. As those skilled in the art will appreciate, other amino acids comprising secondary amino groups may behave similarly. A ProLock™ stapling system incorporates these design principles, and among other things, can reduce the number of amide protons available for interaction with water by, e.g., i) removing the N-terminal amide proton via incorporation of a N-acylated (e.g., N-acetylated) proline or an analog thereof, and ii) stabilizing the proline or an analog thereof in a conformation that cloaks one or more (e.g., two of the three remaining) unsatisfied N-terminal amide protons e.g., through bifurcated hydrogen bonding with the carbonyl of the N-acetyl group. In some embodiments, an analog is an amino acid whose amino group is a secondary amine. In some embodiments, an analog as a residue in a provided peptide has the structure of

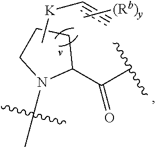

wherein each variable is independently as described herein. In some embodiments, an analog as a residue in a provided peptide has the structure of

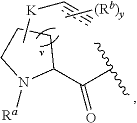

wherein each variable is independently as described herein. In some embodiments, an analog as a residue in a provided peptide has the structure of formula P-I or a salt form thereof. In some embodiments, each heteroatom in a structure of the present disclosure is independently selected from nitrogen, oxygen and sulfur. In some embodiments, Ring A is a monocyclic. In some embodiments, Ring A is saturated. In some embodiments, Ring A is partially unsaturated. In some embodiments, —K— is bonded to —CH— to which R^{a2} is attached to (replacing the H). In some embodiments, R^{a3} is —CH═CH₂ and —C≡CH. In some embodiments, R^{a3} is optionally substituted —CH═CH₂. In some embodiments, R^{a3} is —CH═CH₂. In some embodiments, R^{a3} is optionally substituted —C≡CH. In some embodiments, R^{a3} is —C≡CH. In some embodiments, K is optionally substituted bivalent C₁₋₁₀ alphatic. In some embodiments, K is optionally substituted bivalent C₁₋₁₀ alkylene. In some embodiments, K is linear bivalent C₁₋₁₀ alphatic. In some embodiments, K is linear bivalent C₁₋₁₀ alkylene. In some embodiments, K is optionally substituted bivalent C₁₋₁₀ heteroalphatic having 1-4 heteroatoms. In some embodiments, K is optionally substituted bivalent C₁₋₁₀ heteroalkylene having 1-4 heteroatoms. In some embodiments, K is linear bivalent C₁₋₁₀ heteroalphatic having 1-4 heteroatoms. In some embodiments, K is linear bivalent C₁₋₁₀ heteroalkylene having 1-4 heteroatoms. In some embodiments, a residue has the structure of formula P-II or a salt form thereof, wherein each variable is independently as described herein. In some embodiments, a residue has the structure of formula P-III or a salt form thereof, wherein each variable is independently as described herein. In some embodiments, R^{a} is optionally substituted acyl. In some embodiments, R^{a} is R—C(O)—. In some embodiments, R^{a} is acetyl. In some embodiments, an amino acid has the structure of R^{a}—N(R^{a1})CH(R^{a2})—C(O)OH or a salt thereof. In some embodiments, R^{a3} forms a metathesis product connection, e.g., —CH═CH—, with another double or triple bond of a peptide to form a staple. In some embodiments, Ring A is substituted with —K—R^{a3} (e.g., in certain unstapled peptides, amino acids, etc.). In some embodiments, Ring A is substituted with K, —K—, wherein K is connected to the side chain or backbone carbon of a second amino acid residue optionally through a linker S^{p}.

Various provided compound in the present disclosure may have R^{a}. In some embodiments, R^{a} is H. In some embodiments, R^{a} is optionally substituted acyl. In some embodiments, R^{a} is a suitable amino protecting group. In some embodiments, R^{a} is Fmoc. In some embodiments, R^{a} is t-Boc. As described above, in certain embodiments R^{a} is R—C(O)—. In some embodiments, R^{a} is acetyl.

In some embodiments, a residue of formula P-I has the structure of

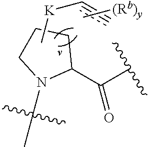

or salt form thereof, wherein each variable is independently as described herein.

In some embodiments, the present disclosure recognizes that carbonyl of an amino acid N-terminal to a proline (or an analog thereof) was sometimes oriented in a conformation that may be capable of forming a bifurcated hydrogen bond with two of the remaining unsatisfied N-terminal amide protons, rather than just one. To confirm the generality of this observation, protein structural data mining was employed. A database containing 42,380 unique protein chain crystal structures was mined to extract all helical peptides with proline at the N-terminus. The extracted helices were those with more than eight amino acids, a proline residue at the N-terminus of helices, and a calculated percent helicity greater than 60%. Briefly, using MOE (see Santiago et al., Current Topics in Medicinal Chemistry 2008, 8 (18), 1555-1572), a total of 31,933 helical peptides were identified with these criteria. Hydrogen-bonding between the acetyl group (i-1) attached to the N-terminus of proline and the amide NH bonds of the i+2 and i+3 residues was determined using MOE modeling software. Hydrogen-bonding energy between an acceptor and a donor is determined in MOE by extended Huckel H-bond model (see Gerber, P. R., Journal of Computer-Aided Molecular Design 1998, 12 (1), 37-51). The model blends Kirchoff's electrostatic theory (for sigma bonds) and conventional p-orbital MO calculations (for pi-systems) to estimate the hydrogen-bonding energy. Based on the hydrogen-bonding energy calculations, 7,680 helices (˜24%) of the helices were determined to exhibit bifurcated H-bond between the acetyl group attached to N-terminus of proline and i+2 and i+3 amide NH bonds. In other words, about 24% of all helices with proline at the N-terminus appear to possess a bifurcated hydrogen bonding pattern between the carbonyl group attached to the nitrogen of proline (which is denoted herein as position i) and the i+2 and i+3 amide protons.

Based on these results, a properly oriented N-acetylated proline analog was recognized by Applicant to be capable of cloaking two of the unsatisfied N-terminal amide protons via a bifurcated hydrogen bond, thereby interfering with their interaction with water molecules. Among other things, the present disclosure provides peptides comprising such residues, e.g., N-acylated proline such as N-acetyl proline, —C(O)—N(R^{a1})CH(R^{a2})—C(O)—, R—C(O)—N(R^{a1})CH(R^{a2})—C(O)—, R^{a}—C(O)—N(R^{a1})CH(R^{a2})—C(O)—, or salt form thereof.

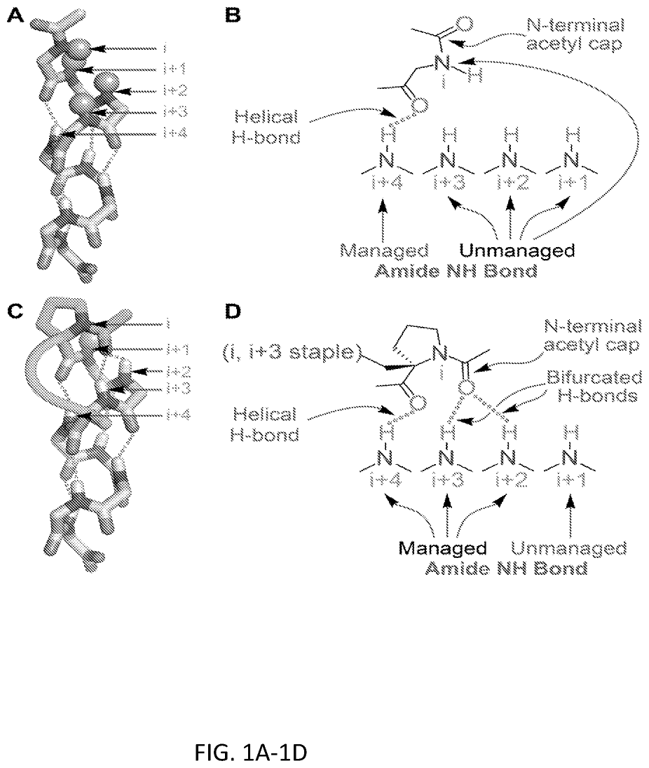

To stabilize an N-acylated proline analog (e.g., N-acylated proline such as N-acetyl proline, —C(O)—N(R^{a1})CH(R^{a2})—C(O)—, R—C(O)—N(R^{a1})CH(R^{a2})—C(O)—, R^{a}—C(O)—N(R^{a1})CH(R^{a2})—C(O)—, or salt form thereof) in such an orientation, a stapling system was incorporated into a peptide. In some embodiments, a staple is a (i, i+3) staple. In some embodiments, an N-acylated proline analog is at position i. In some embodiments, a staple (e.g., a hydrocarbon stapling system) was designed in which a short (i, i-3) staple connects the α-carbon of a proline analog (i) and the α-carbon of an i+3 residue. FIGS. 1A-ID show a ProLock™ stapling system as an example and how it cloaks amide protons at the N-terminus of α-helices. As shown in FIG. 1A, N-acetyl capped helical peptides that are not stapled have four unsatisfied N-terminal amide NH bonds (shown as grey spheres in FIG. 1A). As shown in FIG. 1A, the rest of the amide NH bonds are part of the internal hydrogen bonding network intrinsic to helical peptides. FIG. 1B provides an expanded view of the five N-terminal amide NH bonds in helical peptides of FIG. 1A. As shown in FIG. 1C, various ProLock™ stapled peptides are designed to possess only one unsatisfied amide N-terminal NH bond. As shown in FIG. 1C, the N-terminal amide bond has no attached hydrogen, due to the incorporation of N-acetyl capped proline. The amide NH bonds of the residues at i+2 and i+3 positions are managed by a bifurcated hydrogen bond with the carbonyl oxygen of the N-terminal acetyl cap (see FIG. 1C). The i, i+3 staple originating from the alpha carbon of proline is designed to stabilize the helical fold and propagate helicity towards the C-terminus of the peptide (see FIG. 1C). FIG. 1D provides an expanded view of the five N-terminal amide NH bonds in a ProLock™ stapled peptides of FIG. 1C. Only the amide NH of the i+1 residue remains easily accessible to solvent water (see “unmanaged” i+1 in FIG. 1D).

Thus, as shown schematically in FIGS. 1A-ID, this design is intended to organize the N-terminal amino acids in an α-helical conformation that positions a N-acetyl cap of a proline (or analogs thereof) for optimal hydrogen bonding to the amide protons of the i+2 and i+3 residues. Further, by constraining the N-terminal amino acids in an α-helical conformation, this design is expected to propagate or can be made to form a helical conformation past the stapled i, i+3 region via helix nucleation, as occurs in other α-helical stabilization systems. In sum, a ProLock™ stapling system design comprising an N-terminal proline-based stapling system can reduce the number of unsatisfied N-terminal amide protons from four to one, and can also facilitate the overall peptide in helical conformation.

To construct ProLock™ stapled peptides, a proline amino acid residue analog, PL3, which incorporates an allyl group in the position ordinarily occupied by the hydrogen attached to the proline α-carbon was designed. This PL3 stapling amino acid was designed and synthesized as follows. Those skilled in the art appreciates that other amino acids as described herein may also be utilized, e.g., those having the structure of R^{a}—N(R^{a1})CH(R^{a2})—C(O)OH or a salt thereof.

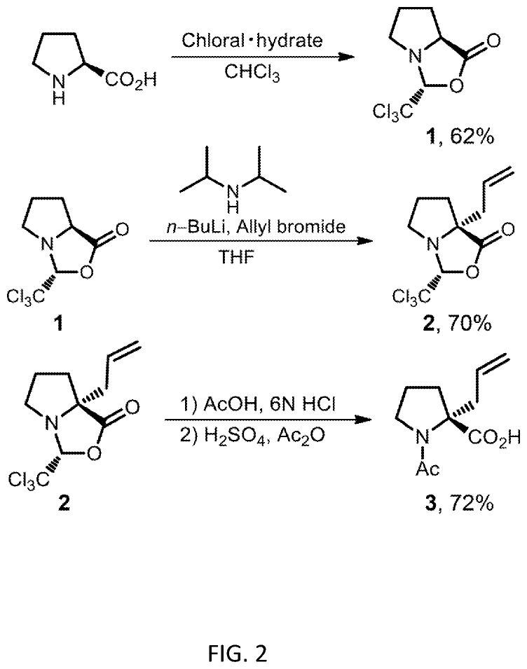

PL3 (Ac-PL3-OH ((R)—N-acetyl-2-(2′-propenyl) proline)) was synthesized in three steps, as has been previously described in PCT Publication No. WO2014/052647 (referred to in that document as “PS3”) and is depicted as compound 3 in the schematic shown in FIG. 2. Briefly (and referring to compounds 1, 2, and 3 in FIG. 2), in the first step, a suspension of L-proline (25.0 g, 217 mmol) and chloral hydrate (54.0 g, 325 mmol) was heated under reflux in chloroform (250 mL) for six hours with a reverse Dean-Stark trap. The solution was washed with water (2×75 mL). The organic layer from the washed solvent was further extracted with chloroform (125 mL). The combined organic layers in chloroform were dried (Na₂SO₄), filtered, and the solvent was removed in vacuo to afford a light brown solid. The crude product was recrystallized from ethanol (200 mL) at 40° C. to afford oxazolidinone (compound 1 in FIG. 2) (32.2 g, 62%) as a white solid. The recrystallized product formed white needles, with the spectroscopic data of compound 1 matched that previously report for similar compounds (see Amedjkouh and Ahlberg, Tetrahedron: Asymmetry 2002, 13 (20), 2229-2234). In the second step, n-Butyl lithium (1.6 M in hexanes, 17.5 mL, 28 mmol) was added dropwise to a stirred solution of diisopropylamine (4.0 mL, 28 mmol) in dry THF (44 mL) at −78° C. under an atmosphere of nitrogen. The solution was stirred for 5 min, warmed to 0° C., and stirred for 15 min. The solution was added dropwise to a solution of oxazolidinone (compound 1) (4.4 g, 17.9 mmol) in dry THF (88 mL) at −78° C. over 20 min, stirred for a further 30 min then allyl bromide (4.9 mL, 56.6 mmol) was added dropwise over 5 min. The solution was warmed to −40° C. and stirred for two hours. Water (66 mL) was added, and the solution was warmed to room temperature and extracted with chloroform (3×175 mL). The combined organic extracts were dried (Na₂SO₄), filtered and evaporated to dryness in vacuo to give a dark brown oil. Purification of the residue by flash column chromatography (5%-10% Ethyl Acetate-Hexanes; gradient elution) afforded oxazolidinone (compound 2) (3.6 g, 70%) as a colorless oil. Spectroscopic data of compound 2 matched that previously reported for similar compounds (see Hoffmann et al., Angewandte Chemie International Edition 2001, 40 (18), 3361-3364). In the final step, a solution of compound 2 (1.50 g, 5.3 mmol) in 65% AcOH-6N HCl (75 mL) was stirred at room temperature for 48 h under a nitrogen atmosphere. The solvent was removed under reduced pressure to give a nearly transparent pale brown oil. The oil was dissolved in acetic anhydride (3.6 mL) and a few drops of concentrated H₂SO₄ was added. The reaction mixture was stirred at room temperature for 24 h, followed by the addition of dichloromethane and water at 0° C. The organic phase was separated, dried by Na₂SO₄, filtered, and evaporated in vacuo. The residue was purified by flash column chromatography (10%-100% Ethyl Acetate-Hexanes+0.1% AcOH; gradient elution) and was recrystallized from Ethyl Acetate-Hexanes to afford compound 3 (0.75 g, 72%) as a white solid. The recrystallized product formed white needles.

Melting Point (Ethyl acetate and hexanes): 107-108° C. ¹H NMR (500 MHz, CDCl₃): 9.41 (br s, 1H), 5.68-5.57 (m, 1H), 5.20-5.12 (m, 2H), 3.65-3.57 (m, 1H), 3.48-3.39 (m, 1H), 2.97 (dd, J=7.0, 7.0 Hz, 1H), 2.76 (dd, J=7.0, 7.0 Hz, 1H), 2.68-2.55 (m, 1H), 2.14 (s, 3H), 2.00-1.84 (m, 3H). ¹³C NMR (125 MHz, CDCl₃): 173.8, 172.9, 131.5, 120.2, 71.6, 50.9, 37.7, 34.3, 23.3, 23.1. FT-IR (neat): 3449, 3014, 2980, 2880, 2360, 2342, 1706, 1604, 1448, 1424, 1245, 1214, 920, 755 cm⁻¹. HRMS (ESI⁺) Calculated for C₁₀H₁₆NO₃: 198.1125. Found: 198.1129.

Fmoc-PL3-LeucicAcid-OH ((S)-2-(((R)-1-(((9H-fluoren-9-yl)methoxy) carbonyl)-2-allylpyrrolidine-2-carbonyl)oxy)-4-methylpentanoic acid) was synthesized in two steps. In the first step, Fmoc-PL3-OH (5 g, 13.25 mmol) was dissolved in 15 mL of dry dichloromethane by stirring at room temperature under an atmosphere of nitrogen. A drop of dimethylformamide (DMF) was added to the solution before adding oxalyl chloride (1.14 mL, 13.25 mmol) in a dropwise manner to the solution. The solution turned yellow immediately after the addition of oxalyl chloride. The reaction was stirred at room temperature for 4 hours. The dichloromethane solvent and unreacted oxalyl chloride were removed in vacuo to afford a pale-yellow oil. In the second step, a solution of (S)-2-hydroxy-4-methylpentanoic acid (1.75 g, 13.25 mmol), 4-dimethylaminopyridine (0.16 g, 1.325 mmol) and N-ethyl-N-isopropylpropan-2-amine (13.85 mL, 79.5 mmol) in 15 mL of dry dichloromethane was added to the crude product from the previous step dissolved in 15 mL of dry dichloromethane by stirring at room temperature under atmosphere of nitrogen. The mixture was stirred for 16 hours at room temperature. The solvent was removed in vacuo before subjecting the mixture to reverse phase purification using Biotage Isolera (40-100% Water-Acetonitrile with 0.1% trifluoroacetic acid gradient was used to purify the product). The final product with trace impurities was lyophilized and used for depsi-linked ProLock stapled peptide synthesis (1.45 g, 29%).

¹H NMR (500 MHz, d6-DMSO): 13.05 (br s, 1H), 7.90 (dd, J=7.5, 1.0 Hz, 2H), 7.68-7.61 (m, 2H), 7.45-7.26 (m, 4H), 5.62 (dddd, J=16.9, 10.1, 7.9, 6.6 Hz, 1H), 5.14 (dd, J=2.5, 1.3 Hz, 1H), 5.11 (dt, J=7.4, 2.6 Hz, 2H), 4.88-4.79 (m, 1H), 4.66 (ddd, J=10.9, 9.6, 4.7 Hz, 1H), 4.18 (t, J=4.9 Hz, 1H), 3.55 (ddd, J=10.1, 7.7, 4.7 Hz, 1H), 3.29 (dd, J=10.2, 7.5 Hz, 1H), 3.06-2.98 (m, 1H), 2.58 (m, 1H), 2.26-1.95 (m, 2H), 1.85-1.75 (m, 1H), 1.78-1.69 (m, 2H), 1.71-1.51 (m, 2H), 0.93-0.82 (m, 6H).

The final PL3 amino acid residue analog (compound 3 in FIG. 2) has the following structure:

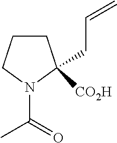

This design permitted the synthesis of staples with varying length and stereochemistry by pairing PL3 with suitable amino acid residues including commercially available hydrocarbon stapling amino acids that bear terminal olefin groups, and cross-linking the two via ring-closing metathesis (RCM). N-acetyl capped PL3 was synthesized from L-proline in three synthetic steps, with an overall yield of 31%.

To evaluate the performance of a ProLock™ stapling system, model peptides based on natural α-helical peptide binders of Estrogen Receptor Ligand Binding Domain (ER LBD) were synthesized (see Warnmark et al., Journal of Biological Chemistry 2002, 277 (24), 21862-21868; McDevitt et al., Bioorganic & Medicinal Chemistry Letters 2005, 15 (12), 3137-3142; Koide et al., Proceedings of the National Academy of Sciences 2002, 99 (3), 1253; Nettles et al., Nature Chemical Biology 2008, 4 (4), 241-247; Fuchs et al., Journal of the American Chemical Society 2013, 135 (11), 4364-4371).

The ER LBD is derived from the estrogen receptor (ER), a member of the steroid hormone nuclear receptor (SHR) family (Evans, R. M. S Science. 1988; 240(4854):889-895). SHRs have an N-terminal activation domain, a central DNA binding domain, and a C-terminal ligand binding domain (LBD) (Tsai and O'Malley, Annu. Rev. Biochem. 1994; 63:451-486). As with other steroid receptors, ER functions as a transcription factor when bound to its cognate agonist by dissociating from chaperone proteins and binding target DNA or other proteins involved in gene transcription (Beato et al., Cell 1995; 83(6):851-857; Brzozowski et al., Nature 1997; 389(6652):753-758).

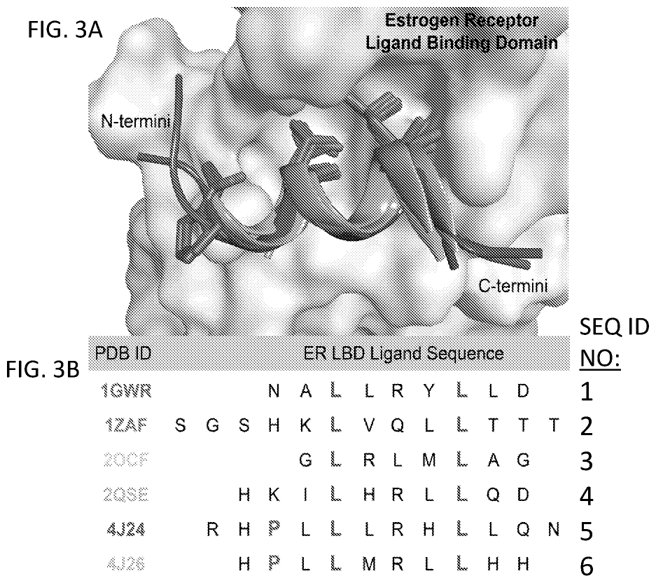

The natural α-helical peptide binders of ER LBD are short, 9-12 amino acid long peptides that have been extensively studied in the context of inhibiting protein-protein interactions (PPIs) of the ER LBD with nuclear receptor coactivator proteins (Speltz et al., Angewandte Chemie International Edition 2016, 55 (13), 4252-4255; Phillips et al., Journal of the American Chemical Society 2011, 133 (25), 9696-9699). These peptides were found to be attractive for assessing a ProLock™ stapling system for two main reasons. First, most of these peptides contain a pair of conserved leucine residues but there is significant diversity in the remaining amino acids within the sequences, resulting in a range of charge and polarity. FIG. 3A provides an overlay of α-helical peptide ligand structures and sequences in complex with the Estrogen Receptor Ligand Binding Domain (ER LBD). The two conserved leucine residues of the six peptide binders are shown with blue sticks in FIG. 3A, and used to align the sequences of the peptides in FIG. 3B. As shown in FIG. 3B, two of the six sequences, namely those with Protein Database ID Nos. 4J24 and 4J26, possess a native proline, shown with dark grey sticks in FIG. 3A, towards the N-terminus of the helix. The variety in the natural α-helical peptide binders of the ER LBD offered the opportunity to assess the generality of ProLock™ stapling system performance in peptides with varying physicochemical properties. Second, a number of known ER LBD binders have an N-terminal proline in their sequences. Inspection of co-crystal structures of these peptides bound to the ER LBD indicated that a hydrocarbon staple connecting the PL3 proline analog to the i+3 position would be largely solvent-exposed. This suggested that incorporation of a ProLock™ staple at those positions could be tolerated in the context of binding to the ER LBD.

Based on the sequences of known ER LBD binders, an initial panel of ten ProLock™ stapled peptides was designed and tested for passive permeability through an artificial membrane (to reflect passage through a cell membrane) using the PAMPA assay to obtain the PAMPA effective permeability (Pₑ), as well as the half maximal effective concentration (EC₅₀) for binding to the ER LBD, and hydrophobicity (CHI Log D).

For peptide synthesis, methyl indole AM resin (EMD Millipore; Burlington, MA) was used as the resin to synthesize peptides with a C-terminal methyl amide cap. ProTide Rink Amide resin (CEM Corporation) was used as the resin to synthesize peptides with a C-terminal amide cap. Peptides were synthesized by standard Fmoc-based solid-phase peptide synthesis (Fmoc-SPPS), according to standard methods (see Verdine and Hilinski, “Stapled peptides for intracellular drug targets”. In Methods in Enzymology. Protein Engineering for Therapeutics, Vol 203, Pt B, Wittrup, K. D.; Verdine, G. L., Eds. Elsevier Academic Press Inc: San Diego, 2012; Vol. 503, pp 3-33). Ring-closing metathesis reaction was performed twice with 25 mol % of Grubb's-I catalyst at 37° C. for 2 hours unless specified otherwise. The peptides were purified by reverse-phase semi-preparatory HPLC (Agilent) with a Zorbax C8 semi-prep column (Agilent) with water and acetonitrile with 0.1% formic acid gradient. The peptides were analyzed by HPLC-MS (Agilent) using a reverse phase C18 column (Phenomenex).

A split-pool ProLock stapled library was designed to test the peptides in library format, as shown in Table 1 below.

TABLE 1
Ac - PL3 - X1 - Leu - S5 - X2 - Leu - Leu - X3 - X4 - NHMe

| | | | |
|---|---|---|---|
| Leu | Arg | Asp | Asp |
| Thr | His | Asn | Asn |
| Ala | Tyr | Gln | Tyr |
| Cpa | Cpa | Thr | Blank |
| KM2 | RM2 | His | |

As shown in Table 1, four positions (X1 through X4) in the base peptide sequence were mutated with the natural and unnatural amino acid residues listed, where Cpa is cyclopropyl alanine; KM2 is N, N′ dimethyl lysine; and RM2: symmetric dimethyl arginine. The library was designed to have peptides which are eight- and nine-residue in length.

Thus, the split-pool ProLock™ stapled peptide libraries were synthesized using Methyl Indole AM resin (EMD Millipore). The split-pool synthesis followed standard Fmoc-SPPS except at the variable positions shown in Table 1. At a variable position, the resin was split into different reaction vessels after Fmoc-deprotection and followed by the coupling of multiple amino acids. The resin was then pooled together following the coupling step before proceeding further. Following cleavage from resin, a ProLock™ stapled peptide library was purified by plate-based C18 cartridge (Sep-Pak) purification before proceeding further analysis and characterization.

The synthesized peptides were first analyzed with a PAMPA assay. PAMPA (Parallel Artificial Membrane Permeability Assay) is an in vitro assay used to measure the passive permeability of compounds (Avdeef, A., Expert Opinion on Drug Metabolism & Toxicology 2005, 1 (2), 325-342). The PAMPA plate system is composed of two compartments, donor and acceptor, which are separated by a phospholipid-infused membrane. The amount of compound transferred from the acceptor well into the donor well over a period of incubation time is used to determine the passive permeability of a compound.

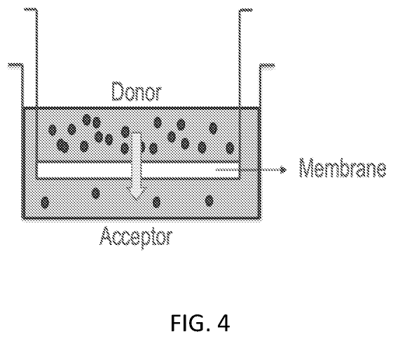

PAMPA assays were typically run in a 96-well format with the membrane-containing donor compartment (donor plate) sitting inside the bottom acceptor compartment (acceptor plate) (see a typical PAMPA well in FIG. 4). The two plates are separated by a membrane infused with phospholipids. The amount of compound transferred from the acceptor well into the donor well over a period of incubation is used to determine the effective permeability (Pe) of a compound (e.g., a stapled peptide). The amount of compound transferred from one well to the other is usually quantified by reverse-phase HPLC-mass spectrometry. The PAMPA assay has been extensively used to determine the passive permeability of small molecules, and the assay has been adapted for measuring the passive permeability of peptide macrocycles (Hewitt et al., Journal of the American Chemical Society 2015, 137 (2), 715-721; Wang et al., Proc Natl Acad Sci USA 2014, 111 (49), 17504-17509; Hickey et al., Journal of Medicinal Chemistry 2016, 59 (11), 5368-5376; Rader et al., Bioorganic & Medicinal Chemistry 2018, 26 (10), 2766-2773).

Cell permeability was one of the properties desired by the stapled peptides described herein, thus, peptides with a higher Pe value were sought. Briefly, all PAMPA assays were performed using the commercially available PAMPA plate system from Corning, which uses a proprietary membrane composed of structured phospholipids. The plastic PAMPA plates themselves are made with polystyrene, which strongly binds to peptides and thus complicates peptide-based PAMPA assays because the retention of peptides at the end of experiments is low. To prevent nonspecific peptide loss to the plates, the acceptor wells of the Corning PAMPA plates were blocked overnight with 5% nonfat milk with 0.1% Triton X-100 in Dulbecco's phosphate buffer saline (DPBS) at pH=7.4, followed by extensive washing prior to assays. The donor wells and membrane itself were not exposed to the milk/Triton solution. Blocking the polystyrene acceptor plates with the milk/Triton solution results in modest-to-high retention of most ProLock peptides in solution, without affecting permeability values themselves, as both CsA and Warfarin afforded Pe values similar to values reported previously (Chen et al., Pharmaceutical Research 2008, 25 (7), 1511-1520; Rezai et al., Journal of the American Chemical Society 2006, 128 (8), 2510-2511). The PAMPA experiments described herein were performed using plates from Corning Lot #9112010, Lot #9112011 and Lot #7317005 (Corning Inc., Corning, New York, USA).

To set up PAMPA assays, the PAMPA plate system was equilibrated at room temperature for at least an hour before setting up the assay. The acceptor plate preincubated with milk blocking solution was washed with water, followed by DPBS buffer. 300 μL of 5% DMSO in DPBS at pH=7.4 was added to the pre-blocked acceptor plate, and 200 μL of 10M compounds made with 5% DMSO in DPBS at pH=7.4 was added to the donor plate. The donor plate with a lid was then carefully inserted into the acceptor plate with no bubbles formed at the interface of donor and acceptor plates. The PAMPA assay plate was covered with aluminum foil and placed in a 37° C. incubator for 5 hours. 10 M input solution of all compounds was diluted 20-fold to a final acetonitrile concentration of 50% before analyzing by reverse-phase HPLC and Q-Exactive plus mass spectrometer. At the end of 5 hours, PAMPA donor and acceptor plates were separated and diluted 20-fold and 2-fold respectively to a final acetonitrile concentration of 50%. The diluted solutions of PAMPA donor and acceptor plates were analyzed by reverse-phase HPLC and Q-Exactive plus mass spectrometer. The ion intensity of compounds from the MS analysis of input sample, donor and acceptor samples at the end of incubation time were used to measure effective permeability (Pe) and % retention of each compound.

Pe measures the passive permeability of each compound tested in the PAMPA assay, whereas % retention measures the amount of compound retained in solution at the end of the assay compared to the input sample. Since plate-to-plate variation can be observed with this assay, two approved orally bioavailable drugs, Warfarin, Cyclosporine A (CsA), and a few ProLock stapled peptides with modest PAMPA permeability were used as controls on each PAMPA plate to ensure the quality of the assay results. All PAMPA data reported herein were from plates in which the average effective permeability (Pₑ) of Warfarin was between 7×10⁻⁶ cm/sec to 11×10⁻⁶ cm/sec and the Pₑ of CsA was between 0.2×10⁻⁶ cm/sec to 0.5×10⁻⁶ cm/sec, which are similar to values previously reported (see, e.g., Chen et al., Pharmaceutical Research 2008, 25 (7), 1511-1520; Rezai et al., Journal of the American Chemical Society 2006, 128 (8), 2510-2511).

PAMPA analysis of the ten first-generation ProLock™ stapled peptides showed that four peptides, PLL4-4, PLL4-5, PLL4-7, and PLL4-10, were capable of crossing PAMPA membrane with Pₑ values of 0.3×10⁻⁶ cm/sec to 0.7×10⁻⁶ cm/sec (see Table 2 below). While these values are relatively low compared to many orally bioavailable drugs, they are comparable to that of CsA under the same experimental conditions.

In addition to the PAMPA assay, to study the affinity of the first-generation ProLock™ stapled peptides to the Estrogen receptor p ligand-binding domain (ERβ LBD), a fluorescence polarization (FP) binding assay in competition format was used in the presence of the endogenous ERβ LBD ligand estradiol. Competition Fluorescence Polarization (FP) assay was performed on the peptides to determine their half-maximal effective concentration (EC₅₀). To do this, FITC labeled ERL4 peptide (FITC-βAla-His-Pro-Leu-Leu-Nle-Arg-Leu-Leu-Leu-Ser-Pro-CONH2) was synthesized to be used as a fluorescence probe based on existing literature (Fuchs et al., Journal of the American Chemical Society 2013, 135 (11), 4364-4371). Direct FP was performed in black 384-well plates (Corning). The final volume of each assay well was 40 μL with constant concentration of FITC labeled ERL4 peptide (10 nM) and variable concentrations of ERβ LBD protein.

ERβ LBD expression and purification was performed as follows. BL21(DE3) pLysS E. coli cells were transformed with a pET28a+ vector containing ERβ LBD (residues from 261 to 500) along with an N-terminal His6-yBBr-TEV tag. An overnight culture in 10 mL of TB media containing 50 μg/mL kanamycin was prepared from a single bacterial colony. The overnight culture was subsequently added to 1 liter of autoclaved TB media containing 50 μg/mL kanamycin and shaken at 37° C. until an OD of 0.6-0.8 was reached. IPTG was added to the culture and left shaking for 6 hours. Bacterial culture was pelleted and resuspended in lysis buffer. Resuspended bacterial culture was incubated at 0° C. for 10 minutes before sonication at 65% amplitude with 10-sec cycles using a Branson Sonifier. The lysate was then cleared by centrifugation at 8000×g for 20 min. ERβ LBD was purified by IMAC affinity (Ni-NTA resin) purification followed by SEC chromatography (Superdex 200 10/300 increase) with a buffer of 35 mM Tris pH 8.0, 150 mM NaCl, 10% glycerol, 1 mM DTT. The expected molecular weight of the ERβ LBD protein is 30.8 KDa, which molecular weight was confirmed by SDS PAGE analysis. Purified ERβ LBD was concentrated to 90 μM and was stored at −80° C. buffer.

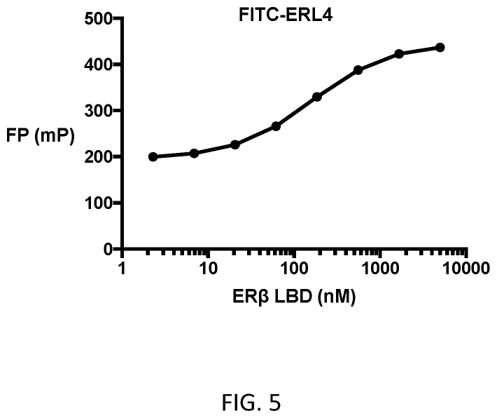

ERβ LBD protein (5 μM) premixed with estradiol (10 μM) was the highest concentration tested in the assay. The subsequent protein concentrations were three-fold dilutions from the highest concentration of 5 μM. Upon addition of samples to each well, the plate was centrifuged for 10 seconds at 3000 rpm and incubated at 4° C. for 1 hour. FP measurements were performed at room temperature with excitation and emission wavelengths at 485 and 535 nm using a Clariostar microplate reader (BMG Labtech). The direct FP results were analyzed by Prism 7 (GraphPad). FIG. 5 shows the direct Fluorescence polarization of FITC-ERL4 probe with ERβ LBD. The K_{D} of the probe in FIG. 5 was determined to be 160 nM with a 1:1 binding model with Hill slope. The FP values shown in FIG. 5 are mean±S.E.M of two independent replicates. Thus, as FIG. 5 shows, the non-linear regression analysis of the direct FP data estimated the K_{d} of FITC labeled ERL4 peptide to be 160 nM with a 1:1 binding model with Hill slope.

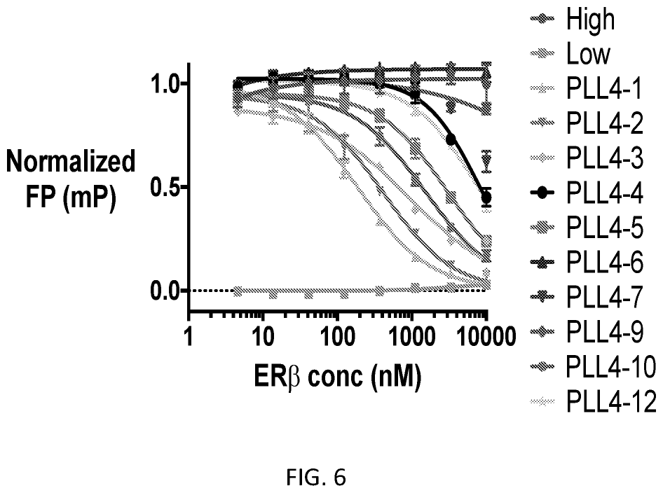

Competition FP assays were also employed. These were performed in black 384-well plates (Corning). The final volume of each assay well was 80 μL containing constant concentrations of estradiol (1 μM) and FITC labeled ERL4 (10 nM). The ERβ LBD concentration was held constant (200 nM) while varying the concentration of ProLock stapled peptides (3-fold dilution from 10 μM). Upon addition of samples to each well, the plate was centrifuged for 10 seconds at 3000 rpm and incubated at 4° C. for 1 hour. FP measurements were performed at room temperature with excitation and emission wavelengths at 485 and 535 nm using a Clariostar microplate reader (BMG Labtech). Each plate had high and low polarization samples, which are used to normalize the assay data. The competition FP results were analyzed by Prism 7 (GraphPad). The non-linear regression analysis of the data was used to determine the half-maximal effective concentration (EC₅₀) of ProLock stapled peptides. Representative competition FP data is shown in FIG. 6. As shown in FIG. 6, the high and low polarization data from each assay plate was used to normalize the data, which were fit to a 1:1 binding model with Hill slope to determine EC₅₀. The normalized FP values shown in FIG. 6 are mean±S.E.M of at least four independent replicates.

As shown in Table 2, three ProLock™ stapled peptides, PLL4-1, PLL4-2, and PLL4-3 showed submicromolar binding towards the ERβ LBD with half-maximal effective concentration (EC₅₀) values of 200 nM, 400 nM, and 700 nM, respectively. As Table 2 shows, the two peptides with detectable PAMPA permeability, PLL4-5 and PLL4-10, showed modest affinity towards the ERβ LBD with EC₅₀ values of 3 μM and 1.5 μM, respectively. These results demonstrate that ProLock stapled peptides can be capable of both target binding and passive membrane permeability.

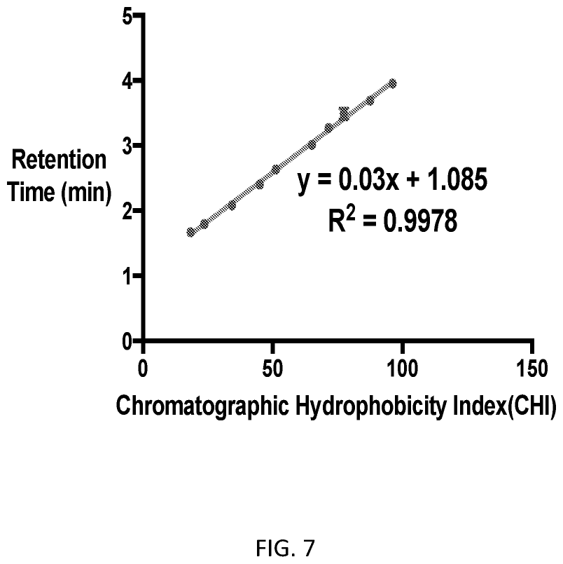

Finally, to measure hydrophobicity, a CHI Log D measurement assay was employed. To do this, a calibration curve to relate chromatographic hydrophobicity index (CHI) based on the retention time(t_{R}) was plotted using ten small molecules used as a standard set (see Valko et al., ADMET & DMPK 2018, 6 (2), 162-175). The curve is shown in FIG. 7, plotting retention time and chromatographic hydrophobicity index (CHI) with ten standard compounds. The retention time measurements were performed using reverse-phase HPLC and Q-Exactive mass spectrometer with Phenomenex Gemini C-18 column (3 uM, 50×3 mm). The reverse-phase chromatography was performed using a gradient of 10 mM ammonium acetate at pH=7.4 and acetonitrile solvents. Using the identical solvent setup, gradient, and flowrate, ProLock stapled peptides were injected onto the column to determine the t_{R} of peptides. Using the calibration curve shown in FIG. 7, t_{R} is converted into CHI. CHI was converted into CHI Log D using the following equation.

CHI Log D=(0.0525×CHI)−1.467

Table 2 below shows the biophysical and biochemical characterization of ten first-generation ProLock™ stapled peptides, having the sequences of SEQ ID Nos: 7 (PLL4-1), 8 (PLL4-2), 9 (PLL4-3), 10 (PLL4-4), 11 (PLL4-5), 12 (PLL4-6), 13 (PLL4-7), 14 (PLL4-9), 15 (PLL4-10), and 16 (PLL4-12), respectively. The sequences were chosen to cover a diversity of aliphatic, aromatic, acidic, and basic amino acids, and to represent all of the commonly used side chain protecting groups for natural amino acids in Fmoc-based solid-phase peptide synthesis (Fmoc SPPS). The stapling amino acid pairs were incorporated at two different registers with respect to the conserved leucine binding residues. PAMPA effective permeability (Pₑ), half maximal effective concentration (EC₅₀) for binding to the ER LBD, and hydrophobicity (CHI Log D) measurements are shown in Table 2. Two orally bioavailable drugs, Warfarin and Cyclosporine A (CsA), were used as controls on each PAMPA plate to ensure assay reproducibility. PAMPA, EC₅₀, and CHI Log D values are mean S.E.M of at least eight, four, and three independent replicates, respectively.

TABLE 2

| | | PAMPA Pₑ | EC₅₀ | CHI LogD |
|---|---|---|---|---|
| Peptide | Sequence | (x10⁻⁶) cm/sec | (μM) | SEM <0.1) |
| PLL4-1 | Ac-PL3-Ile-Leu-S5-Arg-Leu-Leu-Gln- | ≤0.1 | 0.2 ± 0.1 | 2.9 |
| | Tyr-NHMe | | | |
| PLL4-2 | Ac-PL3-Leu-Leu-S5-Arg-His-Leu-Leu- | ≤0.1 | 0.4 ± 0.1 | 2.7 |
| | Tyr-NHMe | | | |
| PLL4-3 | Ac-PL3-Leu-Leu-S5-Arg-Leu-Leu-His- | <0.1 | 0.7 ± 0.7 | 2.7 |
| | Tyr-NHMe | | | |
| PLL4-4 | Ac-PL3-Ala-Leu-S5-Arg-Tyr-Leu-Leu- | 0.5 ± 0.1 | 7.9 ± 0.4 | 2.3 |
| | Asp-NHMe | | | |
| PLL4-5 | Ac-PL3-Lys-Leu-S5-Asn-Leu-Leu-Thr- | 0.7 ± 0.2 | 3.0 ± 0.1 | 1.8 |
| | Tyr-NHMe | | | |
| PLL4-6 | Ac-PL3-Ala-Leu-S5-Leu-Nle-Leu-Ala- | ≤0.1 | NB | 4.0 |
| | Tyr-NHMe | | | |
| PLL4-7 | Ac-PL3-Leu-His-S5-Leu-Leu-Gln-Tyr- | 0.3 ± 0.1 | NB | 2.5 |
| | NHMe | | | |
| PLL4-9 | Ac-PL3-Leu-Nle-S5-Leu-Leu-His-Tyr- | ≤0.1 | NB | 3.3 |
| | NHMe | | | |
| PLL4-10 | Ac-PL3-Leu-Leu-S5-Tyr-Leu-Leu-Asp- | 0.6 ± 0.1 | 1.5 ± 0.1 | 2.4 |
| | NHMe | | | |
| PLL4-12 | Ac-PL3-Leu-Arg-S5-Nle-Leu-Ala-Tyr- | ≤0.1 | 7.5 ± 0.4 | 2.7 |
| | NHMe | | | |
| Warfarin | | 8.6 ± 0.3 | | |
| CsA | | 0.3 ± 0.1 | | 4.8 |

Note that “Ac” in the table above is acetyl group (CH₃C(O)—).

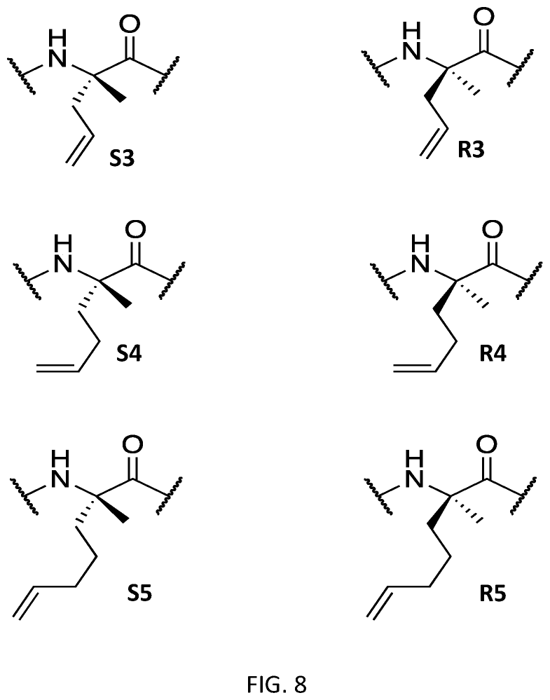

Various ProLock™ stapling amino acid combinations and stapling conditions were further assessed. One of the ten designed peptides, PLL4-5, was used as a model sequence to identify the optimal amino acid at the i-3 position for stapling with PL3, as well as the optimal stapling conditions. Isomer distribution, overall yield, and α-helicity were evaluated. PL3 was fixed as the stapling amino acid at position i, and the length and stereochemistry of stapling amino acid at position i+3 were varied with six different amino acids: S3, S4, S5 and their enantiomers R3, R4, and R5. The structures of the S3, S4, S5, R3, R4, and R5 amino acids are depicted in FIG. 8.

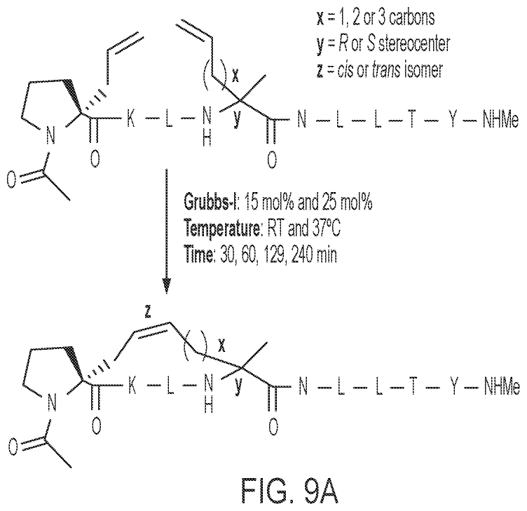

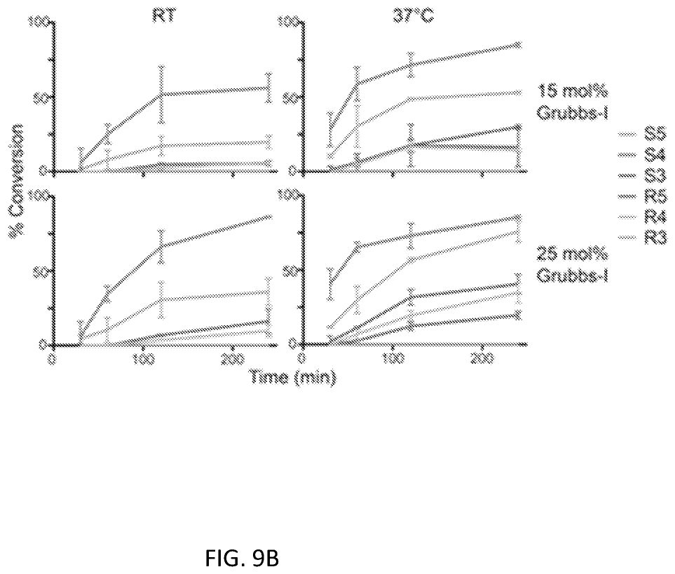

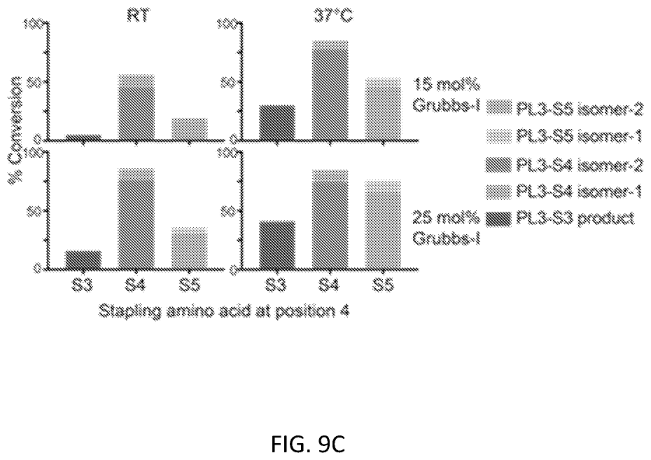

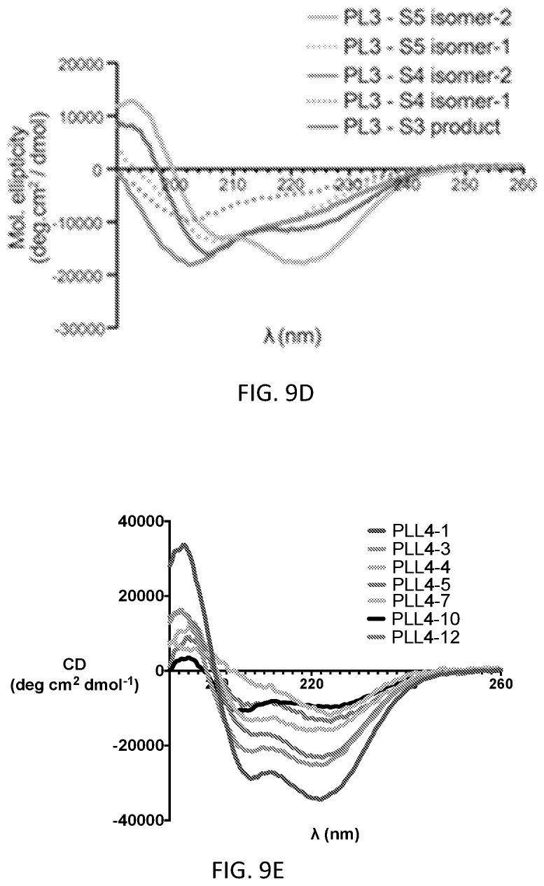

FIG. 9A is a schematic showing how all six peptides were synthesized by Fmoc SPPS and were subjected to ring closing metathesis (RCM) reaction on the solid phase. Briefly, the N-terminal amino acid of a model peptide sequence, PLL4-5, was fixed to N-acetyl PL3, whereas the fourth residue was substituted with six different α-methylated stapling amino acids: S5, S4, and S3 along with their enantiomers R5, R4, and R3. For the reactions in FIG. 9A, the RCM reaction was performed using two different concentrations of Grubbs-I catalyst (15 mol % and 25 mol %), two different temperatures for stapling (room temperature (RT) and 37° C.), and four different reaction times (30, 60, 120, and 240 min). For the 240 min tests, the catalyst was washed off the resin after 120 min and an additional treatment of fresh catalyst was introduced. Following stapling, peptides were cleaved from resin with trifluoroacetic acid (TFA) and analyzed by reverse phase HPLC-MS to assess product conversion yield and isomer distribution.

The results of the reactions are shown in FIG. 9B and suggest that the maximum product was formed at 240 min for most of the staple combinations under all tested conditions. FIG. 9B shows the percent product conversion of all six peptides at two RCM temperatures, two catalyst concentrations and four different timepoints. Treatment with 25% catalyst at 37° C. afforded the highest percent conversion for most of the staple combinations, and in general, a higher product conversion was observed with S-stereoisomers at position i+3 compared to R-stereoisomers (see FIG. 9B). As FIG. 9B shows, the PL3-S4 and PL3-S5 staple combinations showed superior conversion when compared to all other staple combinations. While the PL3-S3 staple combination yielded a single product peak, the PL3-S4 and PL3-S5 combinations generated two different stapled products, as evidenced by two resolvable peaks in HPLC. As the masses of the two products were identical, these peaks were attributed to be the cis- and trans-double-bond isomers, which are commonly observed in other hydrocarbon stapling systems. For each pair of isomers, the stapled product with an earlier retention time was labeled as product isomer 1, whereas the stapled product eluting at a later retention time was labeled as isomer 2. Those skilled in the art appreciate that stapled peptides may be prepared utilizing various suitable methods, including various metathesis catalysts and conditions, in accordance with the present disclosure.

MOE software was used to generate solution NMR structures of PLL4-5 by performing LowMode MD simulations with the distance and dihedral restraints containing structure (Labute, P., Journal of Chemical Information and Modeling 2010, 50 (5), 792-800). The “Protein Consensus” tool in MOE was used to estimate the backbone RMSD of five lowest energy structures.

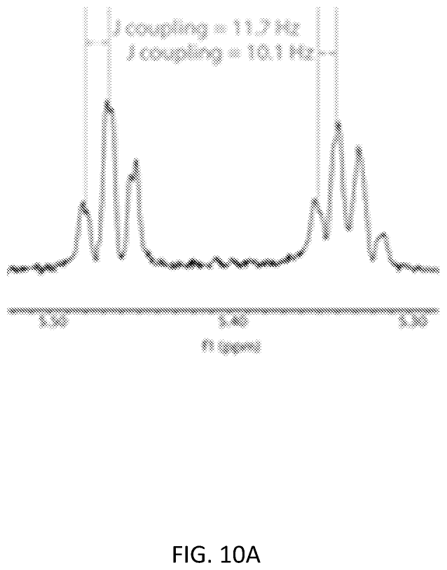

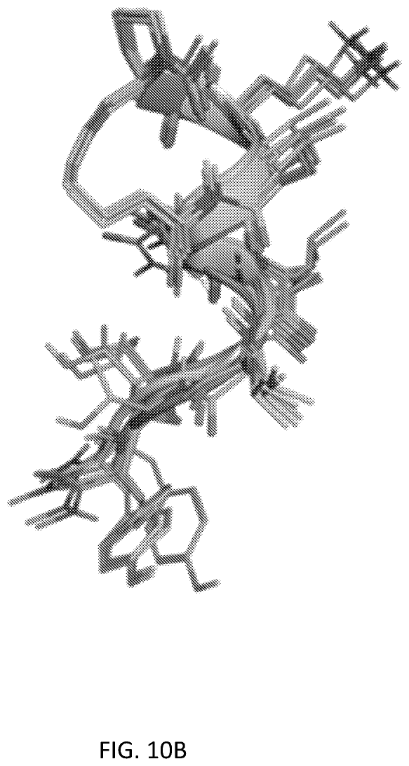

For PLL4-5, the solution NMR analysis shown in FIG. 10A indicated that the isomer 2 product was cis configured, implying that isomer 1 product is trans configured. Both the PL3-S4 and PL3-S5 staple combinations yielded a significantly higher percent conversion of the isomer 2 product (FIG. 9C). FIG. 9C shows the percent product conversion and isomer distribution of three peptides with S3, S4 and S5 stapling amino acids at position 4, at two RCM temperatures, and two catalyst concentrations. The optimal stapling condition based on superior isomer 2 product conversion was with 25% Grubbs-I catalyst at 37° C. for 240 minutes. Due to low percent conversion, stapled products from R-stereoisomers in combination with PL3 were omitted from subsequent experiments.

The secondary structure of all five stapled products from the S-configured i+3 stapling amino acids were analyzed by circular dichroism (CD) spectroscopy, which is an analytical technique that can be applied to study the secondary structure of proteins and peptides. Briefly, CD spectra of ProLock™ stapled peptides were obtained using Jasco J-810 spectropolarimeter with peptide concentration at 50 μM in 10 mM phosphate buffer at pH=7.4. The CD measurements were taken at a fixed temperature of 20° C. with eight scans for each peptide. CD spectra were plotted with mean residue ellipticity (deg cm²/dmol). As shown in FIG. 9D, CD results indicated that PL3-S5 isomer 2 was the only stapled product that adopted an α-helical conformation, as assessed by the presence of minima at 208 nm and 222 nm, which are characteristic of α-helical folds (Greenfield, N. J., Nat. Protoc. 2006, 1 (6), 2876-2890). The CD spectrum of the PL3-S3 stapled product most closely resembled a 3₁₀ helical conformation, which has a similar CD profile as α-helices but with a significantly deeper minimum at 208 nm compared to 222 nm (Toniolo et al., Journal of the American Chemical Society 1996, 118 (11), 2744-2745). The remainder of the stapled products did not have CD profiles that resemble well-defined secondary structures.

Based on these results, the PL3-S5 combination was identified as the optimal stapling pair for efficiently forming α-helical peptides in the context of the PLL4-5 sequence, with optimal stapling conditions of 25% Grubbs-I catalyst at 37° C. for 240 minutes. The initial panel of model ER LBD peptides listed in Table 2 above were synthesized with the PL3-S5 staple combination using these optimized stapling conditions. The stapled isomer 2 product was the major product for all ten first-generation sequences. Most of these isomer 2 peptides afforded CD spectra consistent with an α-helical conformation, demonstrating that helical stabilization of the PL3-S5 combination can occur in different sequence backgrounds (see FIG. 9E). Consequently, the peptides discussed in the remainder of this work are the isomer-2 products of PL3-S5 stapled ProLock™ stapled peptides, unless noted otherwise.

Next, NMR studies were conducted to determine the olefin geometry and solution structure of the PLL4-5 isomer 2 peptide in solution. The solubility of PLL4-5 isomer 2 in 20 mM phosphate buffer at pH 7.4 was determined to be 880 μM, enabling NMR solution structure determination in aqueous buffer. All NMR measurements for the solution structure determination of PLL4-5 ProLock™ stapled peptide isomers were performed on Bruker Avance 800 MHz NMR spectrometer equipped with a 5 mm TCI cryoprobe. Samples were analyzed in standard 5 mm NMR tubes, using 850 μM of peptide dissolved in 500 μL of 10% D2O/90% H2O containing 20 mM phosphate buffer at pH 7.4. Homonuclear 1H, 1H COSY, TOCSY, and ROESY experiments at 293 K were used for complete resonance assignment. Standard pulse programs available in the Bruker library were used for all experiments. WATERGATE method was used to suppress intense H2O resonance during the NMR measurements (Liu et al., Journal of Magnetic Resonance 1998, 132 (1), 125-129). NMR spectra were processed in MestReNova NMR software and imported into CcpNMR Analysis v2.4.2 for peak assignments and to generate distance and dihedral constraints (Vranken et al., Proteins: Structure, Function, and Bioinformatics 2005, 59 (4), 687-696). The assigned ¹H proton chemical shifts of PLL4-5 ProLock™ stapled peptide are shown below in Table 3.

TABLE 3
¹H proton chemical shifts of PLL4-5 peptide

| Residue | H_{N} | H_{α} | H_{β} | H_{γ} | H_{δ} | H_{ε} | Other |
|---|---|---|---|---|---|---|---|
| PL3 | | | 2.04 | 2.06 | 3.83, 3.65 | | H_{allylCH2} - 3.08, |
| | | | | | | | H_{allylCH} - 5.34 |
| Lys | 7.75 | 4.53 | | | | 2.7 | |
| Leu | 7.88 | 3.83 | 1.6 | 1.6 | 0.82 | | |
| S5 | 7.22 | | 2.09 | 0.84 | 1.9 | 5.47 | H_{βCH3} - 1.38 |
| Asn | 7.54 | 4 | 1.55 | | | | H_{δNH2} - 6.88, 7.36 |
| Leu | 7.76 | 4.15 | 1.75 | 1.75 | 0.82 | | |
| Leu | 7.85 | 4.23 | 1.68 | 1.68 | 0.82 | | |
| Thr | 7.72 | 4.07 | 3.97 | 0.92 | | | |
| Tyr | 7.87 | 4.42 | 3.02, 2.84 | | 6.74 | 7.07 | |
| NHMe | 7.52 | | | | | | H_{CH3} - 2.63 |

2D NMR data from COSY, TOCSY and ROESY experiments were used to achieve complete assignment of proton peaks in the ¹H spectrum of PLL4-5 peptide. The presence of continuous i, i+1 NOEs between adjacent amide protons starting from lysine at position two to leucine at position seven support an α-helical fold through at least the first 1.5 turns of the peptide. The lack of NOEs between amide protons of threonine and tyrosine with the adjacent amide protons indicate that the peptide loses its helical structure at the very C-terminal end of the peptide.

The NOE data of the PLL4-5 peptide was translated into 90 distance restraints and 5 dihedral restraints, which were loaded onto a PLL4-5 peptide model using MOE software. A LowMode MD simulation followed by energy minimization of the restrained model peptide generated a database of possible structures. As shown in FIG. 10A, the ¹H spectrum of the PLL4-5 peptide showed well resolved peaks in the amide region of the spectrum. After utilizing the WATERGATE method to suppress the water signal at 4.8 ppm, two distinct sets of olefinic proton peaks were seen between 5.3-5.5 ppm. The J-coupling of spin-spin splitting between two olefinic protons was measured to be 10.1 and 11.7 Hz using both sets of peaks. Based on prior stapled peptide literature, these values suggest that ProLock™ stapled isomer-2 product adopts cis-olefinic geometry (Yuen et al., Chemical Science 2019, 10 (26), 6457-6466).

As shown in FIG. 10B, an ensemble of the five lowest energy structures showed tight agreement of α-helical conformation in the first seven residues, with the last two residues unraveled. These NMR data shown in FIG. 10B indicate that, in PLL4-5, a ProLock™ stapled peptide cap adopts the intended helical structure, consistent with the CD data discussed above.

### Example 2

Design and Characterization of Second-Generation ProLock™ Stapled Peptides

Studies were performed to the affinity and passive membrane permeability of the first-generation ER LBD peptides. To address this, the peptides with the best PAMPA permeability and ERβ LBD binding affinity were merged. Additionally, exploratory pooled PAMPA screens of combinatorial ProLock peptide libraries were performed, from which hits were selected for synthesis and evaluation as individually purified compounds. In total, eighteen sequences were synthesized and assessed for α-helicity, passive membrane permeability, binding affinity to the ER LBD, and column-based hydrophobicity using the methods described in Example 1 above.

Table 4 below shows the biophysical and biochemical characterization of second-generation ProLock stapled peptides. PAMPA effective permeability (Pₑ), half maximal effective concentration (EC₅₀) for binding to the ER LBD, and hydrophobicity (CHI Log D) measurements are shown. Two orally bioavailable drugs, Warfarin and Cyclosporine A (CsA), were used as controls on each PAMPA plate to ensure quality of the assay. PAMPA, EC₅₀, and CHI Log D values are mean±S.E.M of at least eight, four, three independent replicates, respectively.

TABLE 4

| | | PAMPA Pₑ | EC₅₀ | CHI LogD |
|---|---|---|---|---|
| Peptide | Sequence | (x10⁻⁶) cm/sec | (μM) | (SEM <0.1) |
| PLL5-1 | Ac-PL3-Leu-Leu-S5-Tyr-Leu-Leu- | ≤0.1 | 3.9 ± 0.2 | 2.4 |
| | Asp-Tyr-NHMe | | | |
| PLL5-2 | Ac-PL3-Leu-Leu-S5-Tyr-Leu-Leu- | 1.6 ± 0.1 | 1.2 ± 0.1 | 1.7 |
| | Asp-Asp-NHMe | | | |
| PLL5-3 | Ac-PL3-Leu-Leu-S5-Tyr-Leu-Leu- | ≤0.1 | 0.5 ± 0.3 | 3.5 |
| | Gln-Tyr-NHMe | | | |
| PLL7-1 | Ac-PL3-Thr-Leu-S5-Tyr-Leu-Leu- | 2.1 ± 0.4 | NB | 1.0 |
| | Asp-Asp-NHMe | | | |
| PLL7-5 | Ac-PL3-Cpa-Leu-S5-Arg-Leu-Leu- | 0.8 ± 0.2 | 4.1 ± 0.3 | 2.0 |
| | Gln-Asp-NHMe | | | |
| PLL7-7 | Ac-PL3-Cpa-Leu-S5-Tyr-Leu-Leu- | 1.0 ± 0.1 | 0.9 ± 0.1 | 2.0 |
| | Gln-Asp-NHMe | | | |
| PLL7-8 | Ac-PL3-Ala-Leu-S5-Tyr-Leu-Leu-Gln- | 1.4 ± 0.3 | NB | 1.7 |
| | Asp-NHMe | | | |
| PLL7-9 | Ac-PL3-Leu-Leu-S5-Arg-Leu-Leu- | 1.1 ± 0.2 | 1.3 ± 0.1 | 2.1 |
| | His-Asp-NHMe | | | |
| PLL7-11 | Ac-PL3-Ala-Leu-S5-Cpa-Leu-Leu- | 0.9 ± 0.2 | NB | 2.6 |
| | Asn-Asn-NHMe | | | |
| PLL5-4 | Ac-PL3-Ile-Leu-S5-Tyr-Leu-Leu-Asp- | 0.3 ± 0.1 | 1.3 ± 0.1 | 2.4 |
| | NHMe | | | |
| PLL5-5 | Ac-PL3-Thr-Leu-S5-Tyr-Leu-Leu- | 1.4 ± 0.2 | NB | 1.4 |
| | Asp-NHMe | | | |
| PLL5-6 | Ac-PL3-Lys-Leu-S5-Tyr-Leu-Leu- | 1.5 ± 0.2 | NB | 1.2 |
| | Asp-NHMe | | | |
| PLL5-8 | Ac-PL3-Leu-Leu-S5-Arg-Leu-Leu- | 0.9 ± 0.2 | 7.9 ± 0.4 | 2.1 |
| | Asp-NHMe | | | |
| PLL5-10 | Ac-PL3-Leu-Leu-S5-Tyr-Leu-Leu- | ≤0.1 | 1.5 ± 0.2 | 3.8 |
| | Thr-NHMe | | | |
| PLL5-11 | Ac-PL3-Leu-Leu-S5-Tyr-Leu-Leu-His- | ≤0.1 | NB | 3.2 |
| | NHMe | | | |
| PLL5-12 | Ac-PL3-Leu-Leu-S5-Tyr-Leu-Leu- | ≤0.1 | 2.0 ± 0.1 | 3.2 |
| | Gln-NHMe | | | |
| PLL7-20 | Ac-PL3-Ala-Leu-S5-His-Leu-Leu- | 1.3 ± 0.1 | NB | 2.1 |
| | Asn-NHMe | | | |
| PLL7-23 | Ac-PL3-Thr-Leu-S5-Cpa-Leu-Leu- | <0.1 | 2.4 ± 0.1 | 3.2 |
| | Thr-NHMe | | | |
| Warfarin | | 8.6 ± 0.3 | | |
| CSA | | 0.3 ± 0.1 | | 4.8 |

Table 5 below shows the summary of the percentage (%) retention of compounds tested in the PAMPA assay.

TABLE 5

| Peptide | % | Number of | Peptide | % | Number of |
|---|---|---|---|---|---|
| Number | Retention | Replicates | Number | Retention | Replicates |
| PLL4-1 | 75 ± 2 | 8 | PLL4-5 variant 1 | 52 ± 8 | 8 |
| PLL4-2 | 43 ± 2 | 8 | PLL4-5 variant 3 | 78 ± 8 | 16 |
| PLL4-3 | 73 ± 4 | 8 | PLL4-5 variant 4 | 79 ± 3 | 12 |
| PLL4-4 | 71 ± 2 | 8 | PLL4-5 variant 5 | 73 ± 4 | 8 |
| PLL4-5 | 74 ± 3 | 20 | PLL4-10 variant 1 | 66 ± 2 | 8 |
| PLL4-6 | 48 ± 3 | 8 | PLL4-10 variant 2 | 92 ± 10 | 24 |
| PLL4-7 | 46 ± 3 | 8 | PLL4-10 variant 3 | 106 ± 2 | 18 |
| PLL4-9 | 43 ± 3 | 8 | PLL4-10 variant 4 | 74 ± 6 | 12 |
| PLL4-10 | 63 ± 4 | 16 | PLL4-10 variant 5 | 70 ± 2 | 8 |
| PLL4-12 | 42 ± 2 | 8 | PLL5-2 variant 1 | 69 ± 2 | 8 |
| PLL5-1 | 36 ± 1 | 8 | PLL5-2 variant 2 | 47 ± 6 | 8 |
| PLL5-2 | 97 ± 2 | 60 | PLL5-2 variant 5 | 90 ± 3 | 8 |
| PLL5-3 | 83 ± 1 | 8 | PLL7-7 variant 1 | 84 ± 2 | 8 |
| PLL5-4 | 100 ± 1 | 8 | PLL7-7 variant 2 | 64 ± 1 | 8 |
| PLL5-5 | 101 ± 5 | 24 | PLL7-7 variant 3 | 75 ± 1 | 12 |
| PLL5-6 | 105 ± 5 | 24 | PLL7-7 variant 4 | 75 ± 1 | 12 |
| PLL5-8 | 104 ± 3 | 24 | PLL7-7 variant 5 | 84 ± 2 | 8 |
| PLL5-10 | 34 ± 0 | 8 | PLL7-9 variant 1 | 86 ± 2 | 8 |
| PLL5-11 | 89 ± 1 | 8 | PLL7-9 variant 2 | 82 ± 2 | 8 |
| PLL5-12 | 97 ± 1 | 8 | PLL7-9 variant 3 | 98 ± 10 | 12 |
| PLL7-1 | 114 ± 3 | 16 | PLL7-9 variant 4 | 101 ± 24 | 12 |
| PLL7-5 | 109 ± 2 | 16 | PLL7-9 variant 5 | 91 ± 2 | 12 |
| PLL7-7 | 108 ± 3 | 20 | PLL7-20 variant 3 | 76 ± 10 | 12 |
| PLL7-8 | 107 ± 3 | 16 | PLL7-20 variant 4 | 74 ± 6 | 12 |
| PLL7-9 | 85 ± 4 | 20 | PLL7-20 variant 5 | 88 ± 4 | 8 |
| PLL7-11 | 103 ± 2 | 16 | Warfarin | 115 ± 4 | 52 |
| PLL7-20 | 104 ± 2 | 56 | Cyclosporine A | 27 ± 1 | 52 |
| PLL7-23 | 92 ± 2 | 16 | | | |

As Table 4 above shows, eight out of the eighteen second-generation ProLock™ stapled peptides showed PAMPA permeability with Pₑ values exceeding 1×10⁻⁶ cm/sec. The highest value was for PLL7-1, with a Pₑ of 2.1×10⁻⁶ cm/sec. These represent the first helically stabilized peptides with passive membrane permeability reported at such high levels. Two of the second-generation peptides exhibited submicromolar EC₅₀ values for binding the ERβ LBD, and three of the eight peptides with Pₑ values exceeding 1×10⁻⁶ cm/sec showed EC₅₀ values in the 0.8 μM-1.3 μM range (PLL5-2, PLL7-7, and PLL7-9) (see Table 4).

Notably, all eight of the peptides shown in Table 4 with Pₑ values exceeding 1×10⁻⁶ cm/sec possessed at least one charged residue. In this particular series of peptides, most of the permeable peptides possessed either one or two negatively charged aspartate residues towards the C-terminus of the peptide. The peptides with Pₑ values exceeding 1×10⁻⁶ cm/sec exhibited net charges ranging between −2 to neutral (Note that PLL4-5 from the first-generation peptides has a net charge of +1, with a Pₑ of 0.7×10⁻⁶ cm/sec). These data indicate that modest levels of passive permeability can be achieved even for peptides containing multiple charged residues. Furthermore, although arginine residues do appear in some sequences with Pₑ values exceeding 1×10⁶ cm/sec, they are always accompanied by a negatively charged residue at an i+3 or i+4 position, suggesting that charge pairing may be required for arginine-containing sequences to passively transit membranes.

With the identification of ProLock™ stapled peptides with both modest membrane permeability and ERβ LBD affinity, the importance of ProLock™ stapled peptide design components for maintaining passive membrane permeability were investigated. For this work, six ProLock™ stapled peptides with diverse membrane permeability were selected: PLL4-5, PLL4-10, PLL5-2, PLL7-7, PLL7-9, and PLL7-20.

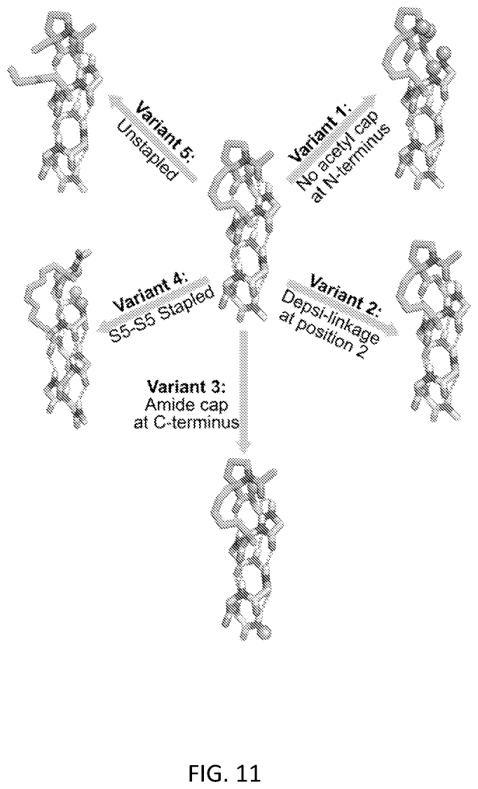

To assess the importance of ProLock™ staple design components, a panel of variants was synthesized for each peptide that systematically removed various features of a ProLock™ stapling system. The features examined as part of this effort were the N-terminal acetyl cap, the remaining solvent-exposed amide proton of the i+1 residue, the C-terminal methyl amide cap, and a ProLock™ staple itself. The corresponding design variants synthesized were: replacement of the N-terminal acetyl cap with no cap (free NH), replacement of the amide linkage at position i+1 with a depsi-linkage (ester bond), replacement of the C-terminal amide cap with no cap (carboxamide C-terminus), incorporation of an S5-S5 staple instead of ProLock™ staple, and omitting olefin metathesis crosslinking to afford an unstapled version of the peptide. FIG. 11 schematically depicts the design variants. The S5-S5 version involved the replacement of the PL3 position with an alanine residue and the incorporation of an S5 residue N-terminal to the alanine (i.e. at the i−1 position). In addition to these modifications, a variant that involved N-methylation of the amide at the i+1 position (to remove the amide proton) was also designed. However, the synthesis and purification of the following peptides were unsuccessful: PL3 could not be coupled to a peptide on resin with an N-methylated terminal residue (even under microwave conditions), and a homogenous product with direct alkylation of fully elongated peptides was unable to be obtained (see White et al., Nature Chemical Biology 2011, 7 (11), 810-817).

Following synthesis and purification, the hydrophobicity (by CHI Log D) of the parent ProLock™ stapled peptides and their variants was examined. Removal of the N-terminal acetyl cap or the C-terminal methyl amide cap reduced Log D, which result was consistent with the introduction of additional polar functionality. Replacement of the amide linkage at position i+1 with a depsi-linkage increased Log D, which result was consistent with the removal of polar functionality. Finally, the use of a longer S5-S5 staple or the omission of olefin metathesis crosslinking to afford an unstapled version increased Log D—these may be due to the introduction of additional hydrocarbon content, although the effects on peptide length, helicity, and the peptide macrodipole complicate this interpretation. The PAMPA results indicated that nearly all of ProLock™ stapled peptide variants possessed inferior passive membrane permeability compared to their original ProLock™ stapled parent peptide. One exception to the above results was the discovery that the PLL7-9 variant with no N-terminal acetyl cap exhibited a surprisingly high CHI Log D compared to the parent ProLock™ stapled peptide (see Table 6 below).

TABLE 6

| | | | | | |
|---|---|---|---|---|---|
| PLL4-5 | PLL4-10 | PLL5-2 | PLL7-7 | PLL7-9 | PLL7-20 |

PAMPA Pₑ (×10⁻⁶) cm/sec

| | | | | | | |
|---|---|---|---|---|---|---|
| Pro-Lock | 0.7 ± 0.2 | 0.6 ± 0.1 | 1.6 ± 0.1 | 1.0 ± 0.1 | 1.1 ± 0.2 | 1.3 ± 0.1 |
| Variant 1 | ≤0.1 | 0.3 ± 0.1 | 0.4 ± 0.1 | 0.4 ± 0.1 | ≤0.1 | |
| Variant 2 | | ≤0.1 | ≤0.1 | ≤0.1 | 0.3 ± 0.1 | |
| Variant 3 | 0.4 ± 0.1 | 0.3 ± 0.1 | | 0.5 ± 0.1 | 0.5 ± 0.1 | 0.5 ± 0.2 |
| Variant 4 | ≤0.1 | ≤0.1 | | ≤0.1 | ≤0.1 | ≤0.1 |
| Variant 5 | ≤0.1 | 0.2 ± 0.1 | 1.7 ± 0.4 | 0.3 ± 0.1 | 0.5 ± 0.1 | 0.3 ± 0.1 |

CHI LogD (SEM <0.1)

| | | | | | | |
|---|---|---|---|---|---|---|
| Pro-Lock | 1.8 | 2.4 | 1.7 | 2.0 | 2.1 | 2.1 |
| Variant 1 | 1.5 | 2.2 | 1.4 | 1.8 | 3.7 | |
| Variant 2 | | 3.0 | 1.9 | 2.6 | 2.4 | |
| Variant 3 | 1.6 | 2.2 | | 1.8 | 1.9 | 2.1 |
| Variant 4 | 2.2 | 2.9 | | 2.4 | 2.7 | 2.6 |
| Variant 5 | 2.3 | 2.8 | 1.8 | 2.3 | 2.5 | 2.7 |

Thus, in one out of the 25 peptides tested, the unstapled version of PLL5-2 possessed a slightly higher PAMPA Pₑ value compared to the parent ProLock™ stapled peptide (1.7×10⁻⁶ cm/sec vs 1.6×10⁻⁶ cm/sec, respectively).

Unexpectedly, as shown in Table 6, the PAMPA permeability for the depsi-linked peptides were consistently lower than those of the original ProLock™ staple version. Given that a depsi linkage removes the one remaining unsatisfied amide proton present at the N-terminus of the helix, this trend was surprising, as the elimination of an amide proton might reasonably be expected to improve passive permeability. While the intention to be limited by theory, it is possible that the conformational preferences of a depsi linkage, which differs significantly from those of amide linkages, disrupt the stability and/or amide proton cloaking properties of a ProLock™ stapling system.

These results indicate that various design features of a ProLock™ stapling system are generally necessary to maintain favorable passive membrane permeability. Without the intention to be limited by theory, this supports the underlying hypothesis for improved N-terminal amide proton cloaking of helices via the introduction of an N-terminal proline cap that is conformationally stabilized by a stapling system, e.g., a hydrocarbon stapling system. The synthesis of N-Fmoc-PL3-OH (rather than N-acetyl-PL3-OH) would enable additional tests of ProLock™ staple designs, in particular the replacement of the acetyl group with alternative capping groups that would not be capable of forming bifurcated hydrogen bonds with the i+2 and i+3 amide protons.

Table 7 provides a summary of the peptide names, theoretical and observed m/z, and charge (z) of the different peptides described in Examples 1 and 2. For depsi-linked peptides, the amino acid at position two is shown as Lec (leucic acid).

TABLE 7

| | | | Theoretical | Observed |
|---|---|---|---|---|
| ProLock variant | Sequence | z | m/z | m/z |
| PL3-S5 unstapled | Ac-PL3-Lys-Leu-S5-Asn-Leu-Leu-Thr- | 1 | 1195.7 | 1195.7 |
| | Tyr-NHMe | | | |
| PL3-S5 stapled | Ac-PL3-Lys-Leu-S5-Asn-Leu-Leu-Thr- | 1 | 1167.7 | 1167.6 |
| | Tyr-NHMe | | | |
| PL3-S4 unstapled | Ac-PL3-Lys-Leu-S4-Asn-Leu-Leu-Thr- | 1 | 1181.7 | 1181.6 |
| | Tyr-NHMe | | | |
| PL3-S4 stapled | Ac-PL3-Lys-Leu-S4-Asn-Leu-Leu-Thr- | 1 | 1153.7 | 1154.6 |
| | Tyr-NHMe | | | |
| PL3-S3 unstapled | Ac-PL3-Lys-Leu-S3-Asn-Leu-Leu-Thr- | 1 | 1167.7 | 1167.6 |
| | Tyr-NHMe | | | |
| PL3-S3 stapled | Ac-PL3-Lys-Leu-S3-Asn-Leu-Leu-Thr- | 1 | 1139.7 | 1139.6 |
| | Tyr-NHMe | | | |
| PL3-R5 unstapled | Ac-PL3-Lys-Leu-R5-Asn-Leu-Leu-Thr- | 1 | 1195.7 | 1195.7 |
| | Tyr-NHMe | | | |
| PL3-R5 stapled | Ac-PL3-Lys-Leu-R5-Asn-Leu-Leu-Thr- | 1 | 1167.7 | 1167.6 |
| | Tyr-NHMe | | | |
| PL3-R4 unstapled | Ac-PL3-Lys-Leu-R4-Asn-Leu-Leu-Thr- | 1 | 1181.7 | 1181.6 |
| | Tyr-NHMe | | | |
| PL3-R4 stapled | Ac-PL3-Lys-Leu-R4-Asn-Leu-Leu-Thr- | 1 | 1153.7 | 1154.6 |
| | Tyr-NHMe | | | |
| PL3-R3 unstapled | Ac-PL3-Lys-Leu-R3-Asn-Leu-Leu-Thr- | 1 | 1167.7 | 1167.6 |
| | Tyr-NHMe | | | |
| PL3-R3 stapled | Ac-PL3-Lys-Leu-R3-Asn-Leu-Leu-Thr- | 1 | 1139.7 | NA |
| | Tyr-NHMe | | | |
| PLL4-1 | Ac-PL3-Ile-Leu-S5-Arg-Leu-Leu-Gln-Tyr- | 2 | 611.4 | 611.5 |
| | NHMe | | | |
| PLL4-2 | Ac-PL3-Leu-Leu-S5-Arg-His-Leu-Leu- | 2 | 615.9 | 616.0 |
| | Tyr-NHMe | | | |
| PLL4-3 | Ac-PL3-Leu-Leu-S5-Arg-Leu-Leu-His- | 2 | 615.9 | 615.9 |
| | Tyr-NHMe | | | |
| PLL4-4 | Ac-PL3-Ala-Leu-S5-Arg-Tyr-Leu-Leu- | 2 | 583.9 | 583.9 |
| | Asp-NHMe | | | |
| PLL4-5 | Ac-PL3-Lys-Leu-S5-Asn-Leu-Leu-Thr- | 1 | 1167.7 | 1167.7 |
| | Tyr-NHMe | | | |
| PLL4-6 | Ac-PL3-Ala-Leu-S5-Leu-Nle-Leu-Ala- | 2 | 540.3 | 540.0 |
| | Tyr-NHMe | | | |
| PLL4-7 | Ac-PL3-Leu-His-S5-Leu-Leu-Gln-Tyr- | 2 | 545.3 | 545.0 |
| | NHMe | | | |
| PLL4-9 | Ac-PL3-Leu-Nle-S5-Leu-Leu-His-Tyr- | 2 | 537.8 | 537.5 |
| | NHMe | | | |
| PLL4-10 | Ac-PL3-Leu-Leu-S5-Tyr-Leu-Leu-Asp- | 1 | 1052.6 | 1052.6 |
| | NHMe | | | |
| PLL4-12 | Ac-PL3-Leu-Arg-S5-Nle-Leu-Ala-Tyr- | 1 | 1051.7 | 1051.6 |
| | NHMe | | | |
| PLL5-1 | Ac-PL3-Leu-Leu-S5-Tyr-Leu-Leu-Asp- | 2 | 608.4 | 608.5 |
| | Tyr-NHMe | | | |
| PLL5-2 | Ac-PL3-Leu-Leu-S5-Tyr-Leu-Leu-Asp- | 1 | 1167.7 | 1167.6 |
| | Asp-NHMe | | | |
| PLL5-3 | Ac-PL3-Leu-Leu-S5-Tyr-Leu-Leu-Gln- | 2 | 614.9 | 615.0 |
| | Tyr-NHMe | | | |
| PLL5-4 | Ac-PL3-Ile-Leu-S5-Tyr-Leu-Leu-Asp- | 2 | 526.8 | 527.0 |
| | NHMe | | | |
| PLL5-5 | Ac-PL3-Thr-Leu-S5-Tyr-Leu-Leu-Asp- | 2 | 520.8 | 521.0 |
| | NHMe | | | |
| PLL5-6 | Ac-PL3-Lys-Leu-S5-Tyr-Leu-Leu-Asp- | 2 | 534.3 | 534.4 |
| | NHMe | | | |
| PLL5-8 | Ac-PL3-Leu-Leu-S5-Arg-Leu-Leu-Asp- | 2 | 523.3 | 523.5 |
| | NHMe | | | |
| PLL5-10 | Ac-PL3-Leu-Leu-S5-Tyr-Leu-Leu-Thr- | 2 | 519.8 | 519.9 |
| | NHMe | | | |
| PLL5-11 | Ac-PL3-Leu-Leu-S5-Tyr-Leu-Leu-His- | 2 | 537.8 | 538.0 |
| | NHMe | | | |
| PLL5-12 | Ac-PL3-Leu-Leu-S5-Tyr-Leu-Leu-Gln- | 1 | 1065.7 | 1065.6 |
| | NHMe | | | |
| PLL7-1 | Ac-PL3-Thr-Leu-S5-Tyr-Leu-Leu-Asp- | 1 | 1155.6 | 1155.6 |
| | Asp-NHMe | | | |
| PLL7-5 | Ac-PL3-Cpa-Leu-S5-Arg-Leu-Leu-Gln- | 2 | 586.4 | 586.4 |
| | Asp-NHMe | | | |
| PLL7-7 | Ac-PL3-Cpa-Leu-S5-Tyr-Leu-Leu-Gln- | 2 | 589.8 | 589.9 |
| | Asp-NHMe | | | |
| PLL7-8 | Ac-PL3-Ala-Leu-S5-Tyr-Leu-Leu-Gln- | 1 | 1138.7 | 1138.6 |
| | Asp-NHMe | | | |
| PLL7-9 | Ac-PL3-Leu-Leu-S5-Arg-Leu-Leu-His- | 2 | 591.9 | 592.0 |
| | Asp-NHMe | | | |
| PLL7-11 | Ac-PL3-Ala-Leu-S5-Cpa-Leu-Leu-Asn- | 1 | 1071.7 | 1071.7 |
| | Asn-NHMe | | | |
| PLL7-20 | Ac-PL3-Ala-Leu-S5-His-Leu-Leu-Asn- | 2 | 492.3 | 492.4 |
| | NHMe | | | |
| PLL7-23 | Ac-PL3-Thr-Leu-S5-Cpa-Leu-Leu-Thr- | 2 | 487.8 | 488 |
| | NHMe | | | |
| PLL4-5 variant-1 | H-PL3-Lys-Leu-S5-Asn-Leu-Leu-Thr-Tyr- | 2 | 563.4 | 563.6 |
| | NHMe | | | |
| PLL4-5 variant-3 | Ac-PL3-Lys-Leu-S5-Asn-Leu-Leu-Thr- | 2 | 577.4 | 577.4 |
| | Tyr-NH2 | | | |
| PLL4-5 variant-4 | Ac-S5-Ala-Lys-Leu-S5-Asn-Leu-Leu-Thr- | 2 | 620.9 | 620.97 |
| | Tyr-NHMe | | | |
| PLL4-5 variant-5 | Ac-PL3-Lys-Leu-S5-Asn-Leu-Leu-Thr- | 2 | 598.4 | 598.5 |
| | Tyr-NHMe | | | |
| PLL4-10 variant- | H-PL3-Leu-Leu-S5-Tyr-Leu-Leu-Asp- | 1 | 1010.6 | 1010.6 |
| 1 | NHMe | | | |
| PLL4-10 variant- | Ac-PL3-Lec-Leu-S5-Tyr-Leu-Leu-Asp- | 1 | 1052.6 | 1052.8 |
| 2 | NHMe | | | |
| PLL4-10 variant- | Ac-PL3-Leu-Leu-S5-Tyr-Leu-Leu-Asp- | 1 | 1038.6 | 1038.8 |
| 3 | NH2 | | | |
| PLL4-10 variant- | Ac-S5-Ala-Leu-Leu-S5-Tyr-Leu-Leu-Asp- | 1 | 1125.7 | 1126.11 |
| 4 | NHMe | | | |
| PLL4-10 variant- | Ac-PL3-Leu-Leu-S5-Tyr-Leu-Leu-Asp- | 1 | 1080.7 | 1080.6 |
| 5 | NHMe | | | |
| PLL5-2 variant-1 | H-PL3-Leu-Leu-S5-Tyr-Leu-Leu-Asp- | 1 | 1125.7 | 1125.6 |
| | Asp-NHMe | | | |
| PLL5-2 variant-2 | Ac-PL3-Lec-Leu-S5-Tyr-Leu-Leu-Asp- | 1 | 1167.6 | 1167.9 |
| | Asp-NHMe | | | |
| PLL5-2 variant-5 | Ac-PL3-Leu-Leu-S5-Tyr-Leu-Leu-Asp- | 1 | 1195.7 | 1195.6 |
| | Asp-NHMe | | | |
| PLL7-7 variant-1 | H-PL3-Cpa-Leu-S5-Tyr-Leu-Leu-Gln- | 2 | 568.8 | 569 |
| | Asp-NHMe | | | |
| PLL7-7 variant-2 | Ac-PL3-Lec-Leu-S5-Tyr-Leu-Leu-Gln- | 2 | 591.3 | 591.6 |
| | Asp-NHMe | | | |
| PLL7-7 variant-3 | Ac-PL3-Cpa-Leu-S5-Tyr-Leu-Leu-Gln- | 2 | 582.8 | 582.92 |
| | Asp-NH2 | | | |
| PLL7-7 variant-4 | Ac-S5-Ala-Cpa-Leu-S5-Tyr-Leu-Leu-Gln- | 2 | 626.4 | 626.82 |
| | Asp-NHMe | | | |
| PLL7-7 variant-5 | Ac-PL3-Cpa-Leu-S5-Tyr-Leu-Leu-Gln- | 2 | 603.9 | 604 |
| | Asp-NHMe | | | |
| PLL7-9 variant-1 | H-PL3-Leu-Leu-S5-Arg-Leu-Leu-His- | 2 | 570.9 | 571 |
| | Asp-NHMe | | | |
| PLL7-9 variant-2 | Ac-PL3-Lec-Leu-S5-Arg-Leu-Leu-His- | 2 | 592.4 | 592.5 |
| | Asp-NHMe | | | |
| PLL7-9 variant-3 | Ac-PL3-Leu-Leu-S5-Arg-Leu-Leu-His- | 2 | 584.9 | 584.91 |
| | Asp-NH2 | | | |
| PLL7-9 variant-4 | Ac-S5-Ala-Leu-Leu-S5-Arg-Leu-Leu-His- | 2 | 628.4 | 628.51 |
| | Asp-NHMe | | | |
| PLL7-9 variant-5 | Ac-PL3-Leu-Leu-S5-Arg-Leu-Leu-His- | 2 | 605.9 | 606 |
| | Asp-NHMe | | | |
| PLL7-20 variant- | Ac-PL3-Ala-Leu-S5-His-Leu-Leu-Asn- | 2 | 485.3 | 485.35 |
| 3 | NH2 | | | |
| PLL7-20 variant- | Ac-S5-Ala-Ala-Leu-S5-His-Leu-Leu-Asn- | 2 | 528.8 | 528.89 |
| 4 | NHMe | | | |
| PLL7-20 variant- | Ac-PL3-Ala-Leu-S5-His-Leu-Leu-Asn- | 1 | 1011.6 | 1011.6 |
| 5 | NHMe | | | |
| FITC-ERL4 | FITC-bAla-His-Pro-Leu-Leu-Nle-Arg- | 1 | 1730.9 | 1730.8 |
| | Leu-Leu-Leu-Ser-Pro-NH2 | | | |

In these Examples 1 and 2, the development of a novel stapling system, ProLock™, that stabilizes peptides in an α-helical conformation while also reducing the number of solvent-exposed amide protons at the peptide N-terminus, was described. Incorporation of a ProLock™ staple into biologically relevant sequences endows them with the ability to passively cross membranes at levels comparable to some orally bioavailable drugs, while retaining the ability to bind their protein target with low- or sub-micromolar affinity.

By intrinsically cloaking nearly all the amide protons in the peptide main chain, a ProLock™ stapling system removes the need to devote significant effort to amide bond cloaking or conversion to isosteres, as is often required when attempting to engineer passive membrane permeability into other peptide structures. This discovery enables further studies to center on the role of the sidechains in passive membrane permeability, target binding, and other physiochemical properties. This system provides a valuable foundation for the development of passively permeable inhibitors of undruggable targets.

### Example 3

This example is directed to a stapled peptide comprising both a ProLock™ staple to cloak the N-terminal amide proton as well as a second staple that holds the C-terminal portion of the peptide in a helical conformation. In some embodiments, the first staple (e.g., a ProLock™ staple) and the second staple are attached to the same amino acid residue. Staples that can be second staples are known (see, e.g., PCT Publication Nos. WO2014/159969 and WO2019/051327, and U.S. Pat. No. 10,487,110).

The various tables in the present disclosure (e.g., Table 7) list the sequences of a number of peptides comprising a single ProLock™ staple. Based on the results from ProLock™ stapled peptides, it is expected that a peptide comprising both a ProLock™ staple and a second staple will have superior qualities in terms of ability to traverse a phosphospholipid-infused membrane (e.g., a PAMPA membrane or a cell membrane) or increased affinity for a target (e.g., the ER LBD target), or both.

As described above, in some peptides, adding only a ProLock™ staple may not dramatically increase the ability of the peptide to traverse a phosphospholipid-infused membrane or may not result in a peptide with dramatically increased affinity for its target.

For example, a native peptide may be able to bind its target with an EC50 of, for example, 750 nM. When a ProLock™ staple is added to the peptide, the EC50 may not dramatically decrease (or may, in fact, increase) as compared to the non-stapled peptide because a ProLock™ staple interferes with the binding of the peptide to its target even though the ability of the stapled peptide to traverse a phosphospholipid-infused membrane increases as compared to the non-stapled peptide. In one example, when both a ProLock™ staple and a second staple are added to the peptide, the EC50 is expected to stay the same as or decrease as compared to the EC50 of the peptide having only a ProLock™ staple, or to stay the same as or decrease below the EC50 of the native (i.e., unstapled) peptide. In another example, when both a ProLock™ staple and a second staple are added to the peptide, the PAMPA permeability value is expected to stay the same as or increase as compared to the PAMPA number of the peptide having only a ProLock™ staple, or to stay the same as or increase as compared to the PAMPA value of the native (i.e., unstapled) peptide. While this would be unexpected to those of skill in the art, because of the analyses described herein to study and optimize a ProLock™ staple in stapled peptides, such an improvement with two staples is predicted.

A similar result is expected in terms of cell permeability (e.g., using a PAMPA assay).

The embodiments of the present disclosure described above are intended to be merely exemplary; numerous variations and modifications will be apparent to those skilled in the art. All such variations and modifications are intended to be within the scope of the present disclosure as defined in any appended claims.

1. A peptide comprising an amino acid residue having the structure of P-I:
—N(R^{a1})CH(R^{a2})—C(O)—, (P-I)
or a salt form thereof, wherein:
R^{a1} and R^{a2} are taken together with their intervening atoms to form Ring A;
Ring A is a substituted 3-10 membered saturated or partially unsaturated ring having 0-3 heteroatoms in addition to the nitrogen to which R^{a1} is attached, wherein at least one substituent of the ring is —K—R^{a3}, or —K—, wherein K is connected to the side chain or backbone carbon of a second amino acid residue optionally through a linker S^{p};
each K is independently a covalent bond, or an optionally substituted C₁₋₂₀ aliphatic or heteroaliphatic chain having 1-6 heteroatoms, wherein one or more methylene unit is optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each R^{a3} is independently an optionally substituted group selected from —CH═CH₂ and —C≡CH;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
S^{p} is —S^{p1}—S^{p2}—S^{p3}—, wherein S^{p1} is bonded to K and S^{p3} is bonded to a side chain or backbone carbon of a second amino acid residue;
each of S^{p1}, S^{p2}, and S^{p3} is independently S^{L};
each S^{L} is independently a bond, a substituted or unsubstituted C₁₋₁₀ alkane, a substituted or unsubstituted C₁₋₁₀ alkylene, or an optionally substituted, bivalent C₁-C₂₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each R′ is independently —R, —C(O)R, —CO₂R, or —SO₂R; and
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

2. The peptide of claim 1, wherein the peptide comprises a residue of formula (P-II): —C(O)—N(R^{a1})CH(R^{a2})—C(O)— or a salt form thereof.

3. The peptide of claim 1, wherein the peptide comprises a residue of formula (P-III): R^{a}—N(R^{a1})CH(R^{a2})—C(O)— or a salt form thereof.

4. The peptide of any one of the preceding claims, wherein Ring A is a substituted 3-10 membered saturated or partially unsaturated ring having 0-3 heteroatoms in addition to the nitrogen to which R^{a1} is attached, wherein at least one substituent of the ring is —K—R^{a3}

5. The peptide of claim 4, wherein R^{a3} is —CH═CH₂.

6. The peptide of claim 4, wherein R^{a3} is —C≡CH.

7. The peptide of any one of claims 1-3, wherein Ring A is a substituted 3-10 membered saturated or partially unsaturated ring having 0-3 heteroatoms in addition to the nitrogen to which R^{a1} is attached, wherein at least one substituent of the ring is —K—, wherein K is connected to the side chain or backbone carbon of a second amino acid residue optionally through a linker S^{p}.

8. A peptide comprising 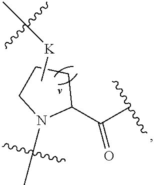
or a salt form thereof, wherein:
v is 1 or 2;
K is a covalent bond, or an substituted or unsubstituted bivalent group selected from a bivalent aliphatic group, alkylene, alkenylene, alkynylene, a bivalent heteroaliphatic group, heteroalkylene, heteroalkenylene, heteroalkynylene, heterocyclene, carbocyclene, arylene, and heteroarylene, and is connected to the side chain or backbone carbon of a second amino acid residue optionally through a linker S^{p};
R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group selected from, or one or more combinations of, substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; and substituted or unsubstituted heteroarylene; or R^{a} is or comprises a peptide moiety;
each instance of R^{b}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
y is 0, 1, 2, or 3;
S^{p} is —S^{p1}—S^{p2}—S^{p3}—, wherein S^{p1} is bonded to K and S^{p3} is bonded to a side chain or backbone carbon of a second amino acid residue;
each of S^{p1}, S^{p2}, and S^{p3} is independently S^{L};
each S^{L} is independently a bond, a substituted or unsubstituted C₁₋₁₀ alkane, a substituted or unsubstituted C₁₋₁₀ alkylene, or an optionally substituted, bivalent C₁-C₂₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

9. A peptide comprising 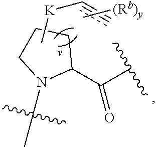
or a salt form thereof, wherein:
v is 1 or 2;
K is a covalent bond, or an substituted or unsubstituted bivalent group selected from a bivalent aliphatic group, alkylene, alkenylene, alkynylene, a bivalent heteroaliphatic group, heteroalkylene, heteroalkenylene, heteroalkynylene, heterocyclene, carbocyclene, arylene, and heteroarylene;
R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group selected from, or one or more combinations of, substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; and substituted or unsubstituted heteroarylene; or R^{a} is or comprises a peptide moiety;
each instance of R^{b}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
y is 0, 1, 2, or 3; and
each instance of [symbol] independently represents a single bond, a double bond or a triple bond.

10. A peptide, comprising an amino acid residue B¹, wherein:
B¹ is B or B′:
B is 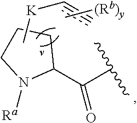
or a salt form thereof,
B′ is 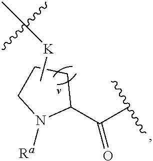
or a salt form thereof,
wherein:
v is 1 or 2;
K is a covalent bond, or an substituted or unsubstituted bivalent group selected from a bivalent aliphatic group, alkylene, alkenylene, alkynylene, a bivalent heteroaliphatic group, heteroalkylene, heteroalkenylene, heteroalkynylene, heterocyclene, carbocyclene, arylene, and heteroarylene, and when B¹ is B′, K is connected to the side chain or backbone carbon of a second amino acid residue optionally through a linker S^{p};
R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group selected from, or one or more combinations of, substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; and substituted or unsubstituted heteroarylene; or R^{a} is or comprises a peptide moiety;
each instance of R^{b}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
y is 0, 1, 2, or 3;
each instance of [symbol] independently represents a single bond, a double bond or a triple bond;
S^{p} is —S^{p1}—S^{p2}—S^{p3}—, wherein S^{p1} is bonded to K and S^{p3} is bonded to a side chain or backbone carbon of a second amino acid residue;
each of S^{p1}, S^{p2}, and S^{p3} is independently S^{L};
each S^{L} is independently a bond, a substituted or unsubstituted C₁₋₁₀ alkane, a substituted or unsubstituted C₁₋₁₀alkylene, or an optionally substituted, bivalent C₁-C₂₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

11. The peptide of claim 1, wherein B¹ is B, and the peptide comprises a second amino acid residue whose side chain comprises an olefin.

12. The peptide of claim 1, wherein B¹ is B, and the peptide comprises a second amino acid residue whose side chain comprises a terminal olefin.

13. The peptide of claim 1, wherein B¹ is B, and the peptide comprises a second amino acid residue J.

14. The peptide of claim 1, wherein B¹ is B′, and B¹ is connected to a second amino acid residue via a staple.

15. The peptide of claim 1, wherein B¹ is B′, and B¹ is connected to a second amino acid J′.

16. The peptide of any one of the preceding claims, wherein there are two amino acid residues between B¹ (at position i) and a second amino acid residue (at position i+3).

17. The peptide of claim 16, where i is 1.

18. A peptide, wherein the peptide is or comprises:
(SEQ ID NO: 1)
B-X²-Z-J-X⁵-X⁶-Z-X⁸-X⁹-X¹⁰-X¹¹-X¹²-X¹³,
or a salt form thereof, wherein:
B is 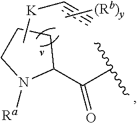
or a salt or a stereoisomeric form thereof, wherein:
v is 1 or 2;
K is a covalent bond, or an substituted or unsubstituted bivalent group selected from a bivalent aliphatic group, alkylene, alkenylene, alkynylene, a bivalent heteroaliphatic group, heteroalkylene, heteroalkenylene, heteroalkynylene, heterocyclene, carbocyclene, arylene, and heteroarylene;
R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group selected from, or one or more combinations of, substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; and substituted or unsubstituted heteroarylene; or R^{a} is or comprises a peptide moiety;
each instance of R^{b}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
y is 0, 1, 2, or 3; and
each instance of [symbol] independently represents a single bond, a double bond or a triple bond;
J is 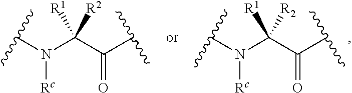
or a salt or a stereoisomeric form thereof, wherein:
each instance of R¹ and R² is independently hydrogen; substituted or unsubstituted aliphatic; substituted or unsubstituted alkylene; substituted or unsubstituted alkynylene; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; or halo; and
each instance of R^{c}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
each instance of Z is independently an amino acid residue which comprises an optionally substituted C₄₋₆ (e.g., C₄, C₅, or C₆) aliphatic side chain, or a leucine amino acid residue or a homolog thereof, such as, for example, a residue selected from the group consisting of a leucine amino acid residue, an isoleucine amino acid residue, a homoleucine amino acid residue, an alloisoleucine amino acid residue, a norleucine amino acid residue, and a tert-leucine amino acid residue, wherein the homolog may be a D stereoisomer or an L stereoisomer;
each of X², X⁵, X⁶, X⁸, X⁹, X¹⁰, X¹¹, X¹², and X¹³ is independently an amino acid residue which may be a D stereoisomer or an L stereoisomer; and
each of X⁹, X¹⁰, X¹¹, X¹², and X¹³ is optionally present.

19. A peptide wherein the peptide is or comprises:
B′-X²-Z-J′-X⁵-X⁶-Z-X⁸-X⁹-X¹⁰-X¹¹-X¹²-X¹³,
or a salt form thereof, wherein:
B′ is 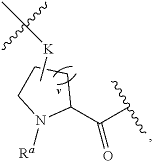
or a salt or a stereoisomeric form thereof, wherein:
v is 1 or 2;
K is a covalent bond, or an substituted or unsubstituted bivalent group selected from a bivalent aliphatic group, alkylene, alkenylene, alkynylene, a bivalent heteroaliphatic group, heteroalkylene, heteroalkenylene, heteroalkynylene, heterocyclene, carbocyclene, arylene, and heteroarylene;
R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group selected from, or one or more combinations of, substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; and substituted or unsubstituted heteroarylene; or R^{a} is or comprises a peptide moiety;
J′ is 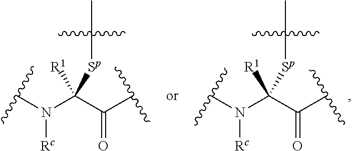
or a salt or a stereoisomeric form thereof;
S^{p} is —S^{p1}—S^{p2}—S^{p3}—, wherein S^{p1} is bonded to K;
each of S^{p1}, S^{p2}, and S^{p3} is independently S^{L};
each S^{L} is independently a bond, a substituted or unsubstituted C₁₋₁₀ alkane, a substituted or unsubstituted C₁₋₁₀ alkylene, or an optionally substituted, bivalent C₁-C₂₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon;
each instance of R¹ is independently hydrogen; substituted or unsubstituted aliphatic; substituted or unsubstituted alkylene; substituted or unsubstituted alkynylene; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; or halo;
each instance of R^{c}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
each instance of Z is independently an amino acid residue which comprises an optionally substituted C₄₋₆ (e.g., C₄, C₅, or C₆) aliphatic side chain, or a leucine amino acid residue or a homolog thereof, such as, for example, a residue selected from the group consisting of a leucine amino acid residue, an isoleucine amino acid residue, a homoleucine amino acid residue, an alloisoleucine amino acid residue, a norleucine amino acid residue, and a tert-leucine amino acid residue, wherein the homolog may be a D stereoisomer or an L stereoisomer;
each of X², X⁵, X⁶, X⁸, X⁹, X¹⁰, X¹¹, X¹², and X¹³ is independently an amino acid residue which may be a D stereoisomer or an L stereoisomer; and
each of X⁹, X¹⁰, X¹¹, X¹², and X¹³ is optionally present.

20. A peptide, wherein the peptide is or comprises:
(SEQ ID NO: 2)
B-Z-X³-J-X⁵-Z-X⁷-X⁸-X⁹-X1⁰-X¹¹-X¹²-X¹³,
or a salt form thereof, wherein:
B is 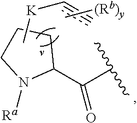
or a salt or a stereoisomeric form thereof, wherein:
v is 1 or 2;
K is a covalent bond, or an substituted or unsubstituted bivalent group selected from a bivalent aliphatic group, alkylene, alkenylene, alkynylene, a bivalent heteroaliphatic group, heteroalkylene, heteroalkenylene, heteroalkynylene, heterocyclene, carbocyclene, arylene, and heteroarylene;
R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group selected from, or one or more combinations of, substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; and substituted or unsubstituted heteroarylene; or R^{a} is or comprises a peptide moiety;
each instance of R^{b}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
y is 0, 1, 2, or 3; and
each instance of [symbol] independently represents a single bond, a double bond or a triple bond;
J is 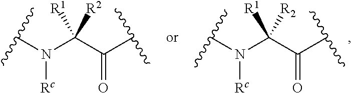
or a salt or a stereoisomeric form thereof, wherein:
each instance of R¹ and R² is independently hydrogen; substituted or unsubstituted aliphatic; substituted or unsubstituted alkylene; substituted or unsubstituted alkynylene; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; or halo; and
each instance of R^{c}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
each instance of Z is independently an amino acid residue which comprises an optionally substituted C₄₋₆ (e.g., C₄, C₅, or C₆) aliphatic side chain, or a leucine amino acid residue or a homolog thereof, such as, for example, a residue selected from the group consisting of a leucine amino acid residue, an isoleucine amino acid residue, a homoleucine amino acid residue, an alloisoleucine amino acid residue, a norleucine amino acid residue, and a tert-leucine amino acid residue, wherein the homolog may be a D stereoisomer or an L stereoisomer;
each of X³, X⁵, X⁷, X⁸, X⁹, X¹⁰, X¹¹, X¹², and X¹³ is independently an amino acid residue which may be a D stereoisomer or an L stereoisomer; and
each of X⁹, X¹⁰, X¹¹, X¹², and X¹³ is optionally present.

21. A peptide, wherein the peptide is or comprises:
(SEQ ID NO: 2)
B′-Z-X³-J′-X⁵-Z-X⁷-X⁸-X⁹-X¹⁰-X¹¹-X¹²-X¹³
or a salt form thereof, wherein:
B′ is 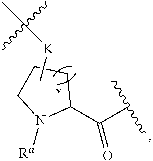
or a salt or a stereoisomeric form thereof, wherein:
v is 1 or 2;
K is a covalent bond, or an substituted or unsubstituted bivalent group selected from a bivalent aliphatic group, alkylene, alkenylene, alkynylene, a bivalent heteroaliphatic group, heteroalkylene, heteroalkenylene, heteroalkynylene, heterocyclene, carbocyclene, arylene, and heteroarylene;
R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group selected from, or one or more combinations of, substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; and substituted or unsubstituted heteroarylene; or R^{a} is or comprises a peptide moiety;
J′ is 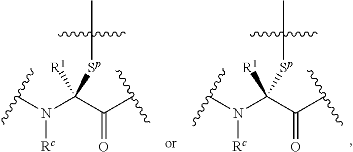
or a salt or a stereoisomeric form thereof;
S^{p} is —S^{p1}—S^{p2}—S^{p3}—, wherein S^{p1} is bonded to K;
each of S^{p1}, S^{p2}, and S^{p3} is independently S^{L};
each S^{L} is independently a bond, a substituted or unsubstituted C₁₋₁₀ alkane, a substituted or unsubstituted C₁₋₁₀ alkylene, or an optionally substituted, bivalent C₁-C₂₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon;
each instance of R¹ is independently hydrogen; substituted or unsubstituted aliphatic; substituted or unsubstituted alkylene; substituted or unsubstituted alkynylene; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; or halo;
each instance of R^{c}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
each instance of Z is independently an amino acid residue which comprises an optionally substituted C₄₋₆ (e.g., C₄, C₅, or C₆) aliphatic side chain, or a leucine amino acid residue or a homolog thereof, such as, for example, a residue selected from the group consisting of a leucine amino acid residue, an isoleucine amino acid residue, a homoleucine amino acid residue, an alloisoleucine amino acid residue, a norleucine amino acid residue, and a tert-leucine amino acid residue, wherein the homolog may be a D stereoisomer or an L stereoisomer;
each of X³, X⁵, X⁷, X⁸, X⁹, X¹⁰, X¹¹, X¹², and X¹³ is independently an amino acid residue which may be a D stereoisomer or an L stereoisomer; and
each of X⁹, X¹⁰, X¹¹, X¹², and X¹³ is optionally present.

22. A peptide, wherein the peptide is or comprises:
B-X²-X³-J-X⁵-X⁶-X⁷-O-X⁹-X¹⁰-X¹¹-X¹²-X¹³,
or a salt thereof, wherein:
B is 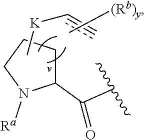
or a salt or a stereoisomeric form thereof, wherein:
v is 1 or 2;
K is a covalent bond, or an substituted or unsubstituted bivalent group selected from a bivalent aliphatic group, alkylene, alkenylene, alkynylene, a bivalent heteroaliphatic group, heteroalkylene, heteroalkenylene, heteroalkynylene, heterocyclene, carbocyclene, arylene, and heteroarylene;
R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; or substituted or unsubstituted heteroarylene; or R^{a} is or comprises a peptide moiety;
each instance of R^{b}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
y is 0, 1, 2, or 3; and
each instance of [symbol] independently represents a single bond, a double bond or a triple bond;
J is 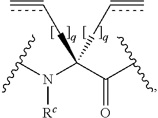
or a salt or a stereoisomeric form thereof, wherein:
each instance of q is independently 1, 2, or 3; and
each instance of R^{c}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
each instance of [symbol] independently represents a single bond, a double bond or a triple bond; and
O is of formula 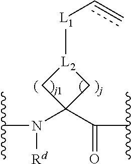
or a salt or stereoisomeric form thereof; wherein:
R^{d} is hydrogen; acyl; substituted or unsubstituted C₁₋₆ alkyl; or an amino protecting group;
each instance of [symbol] independently represents a single bond, a double bond or a triple bond;
L₁ is independently, a bond, a substituted or unsubstituted bivalent C₁₋₁₀ aliphatic or heteroaliphatic, a substituted or unsubstituted C₁₋₁₀ alkylene, —C(O)O—, or —C(═O)OR³—;
L₂ is independently a bond, N, optionally substituted CH, or C(R⁴);
R⁵ is, independently, hydrogen; acyl; substituted or unsubstituted C₁₋₆ alkyl; or an amino protecting group;
each of R³ and R⁴ is independently hydrogen, halogen, —NO₂, —OH, —CN, or C₁₋₆ alkyl;
each of j and j1 is independently 0, 1, 2, 3, 4, 5, 6, 7, 8, 9, or 10; and
each of X², X³, X⁵, X⁶, X⁷, X⁹, X¹⁰, X¹¹, X¹², and X¹³ is independently an amino acid residue which may be a D stereoisomer or an L stereoisomer; and
each of X⁹, X¹⁰, X¹¹, X¹², and X¹³ is optionally present.

23. A peptide, wherein the peptide is or comprises:
B′-X²-X³-J″-X⁵-X⁶-X⁷-O′-X⁹-X¹⁰-X¹¹-X¹²-X¹³,
or a salt thereof, wherein:
B′ is 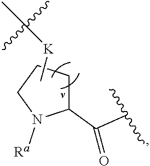
or a salt or a stereoisomeric form thereof, wherein:
v is 1 or 2;
K is a covalent bond, or an substituted or unsubstituted bivalent group selected from a bivalent aliphatic group, alkylene, alkenylene, alkynylene, a bivalent heteroaliphatic group, heteroalkylene, heteroalkenylene, heteroalkynylene, heterocyclene, carbocyclene, arylene, and heteroarylene;
R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group selected from, or one or more combinations of, substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; and substituted or unsubstituted heteroarylene; or R^{a} is or comprises a peptide moiety;
J″ is 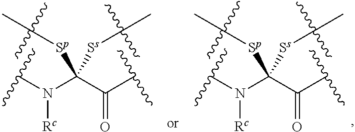
or a salt or a stereoisomeric form thereof, wherein:
S^{p} is —S^{p1}—S^{p2}—S^{p3}—, wherein S^{p1} is bonded to K;
S^{s} is —S^{s1}—S^{s2}—S^{s3}—, wherein S^{s3} is bonded to L₁;
each of S^{p1}, S^{p2} S^{p3}, S^{s1}, S^{s2}, and S3 is independently S^{L};
each S^{L} is independently a bond, a substituted or unsubstituted C₁₋₁₀ alkane, a substituted or unsubstituted C₁₋₁₀alkylene, or an optionally substituted, bivalent C₁-C₂₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon;
O′ is of formula: 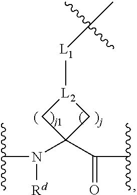
or a salt or stereoisomeric form thereof; wherein:
R^{d} is hydrogen; acyl; substituted or unsubstituted C₁₋₆ alkyl; or an amino protecting group;
L₁ is independently, a bond, a substituted or unsubstituted bivalent C₁₋₁₀ aliphatic or heteroaliphatic, a substituted or unsubstituted C₁₋₁₀ alkylene, —C(O)O—, or —C(═O)OR³—;
L₂ is independently a bond, N, optionally substituted CH, or C(R⁴);
R⁵ is, independently, hydrogen; acyl; substituted or unsubstituted C₁₋₆ alkyl; or an amino protecting group;
each of R³ and R⁴ is independently hydrogen, halogen, —NO₂, —OH, —CN, or C₁₋₆ alkyl;
each of j and j1 is independently 0, 1, 2, 3, 4, 5, 6, 7, 8, 9, or 10; and
each of X², X³, X⁵, X⁶, X⁷, X⁹, X¹⁰, X¹¹, X¹², and X¹³ is independently an amino acid residue which may be a D stereoisomer or an L stereoisomer; and
each of X⁹, X¹⁰, X¹¹, X¹², and X¹³ is optionally present.

24. A peptide wherein the peptide is or comprises:
B-X²-X³-J-X⁵-X⁶-X⁷-X⁸-X⁹-X¹⁰-O-X¹²-X¹³-X¹⁴,
or a salt thereof, wherein:
B is 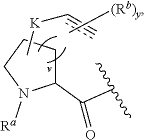
or a salt or a stereoisomeric form thereof, wherein:
v is 1 or 2;
K is a covalent bond, or an substituted or unsubstituted bivalent group selected from a bivalent aliphatic group, alkylene, alkenylene, alkynylene, a bivalent heteroaliphatic group, heteroalkylene, heteroalkenylene, heteroalkynylene, heterocyclene, carbocyclene, arylene, and heteroarylene;
R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group selected from, or one or more combinations of, substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; and substituted or unsubstituted heteroarylene; or R^{a} is or comprises a peptide moiety;
each instance of R^{b}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro;
y is 0, 1, 2, or 3; and
J is 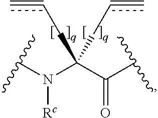
or a salt or a stereoisomeric form thereof, wherein:
each instance of q is independently 1, 2, or 3;
each instance of R^{c}, is, independently, hydrogen; substituted or unsubstituted aliphatic; substituted or unsubstituted heteroaliphatic; substituted or unsubstituted aryl; substituted or unsubstituted heteroaryl; substituted or unsubstituted acyl; substituted or unsubstituted hydroxyl; substituted or unsubstituted thiol; substituted or unsubstituted amino; cyano; isocyano; halo; or nitro; and
each instance of [symbol] independently represents a single bond, a double bond or a triple bond; and
O is of formula 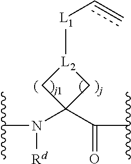
or a salt or stereoisomeric form thereof; wherein:
R^{d} is hydrogen; acyl; substituted or unsubstituted C₁₋₆ alkyl; or an amino protecting group;
each instance of [symbol] independently represents a single bond, a double bond or a triple bond;
L₁ is independently, a bond, a substituted or unsubstituted bivalent C₁₋₁₀ aliphatic or heteroaliphatic, a substituted or unsubstituted C₁₋₁₀ alkylene, —C(O)O—, or —C(═O)OR³—;
L₂ is independently a bond, N, optionally substituted CH, or C(R⁴);
R⁵ is, independently, hydrogen; acyl; substituted or unsubstituted C₁₋₆ alkyl; or an amino protecting group;
each of R³ and R⁴ is independently hydrogen, halogen, —NO₂, —OH, —CN, or C₁₋₆ alkyl;
each of j and j1 is independently 0, 1, 2, 3, 4, 5, 6, 7, 8, 9, or 10; and
each of X², X³, X⁵, X⁶, X⁸, X⁹, X¹⁰, X¹², X¹³, and X¹⁴ is independently an amino acid residue which may be a D stereoisomer or an L stereoisomer; and
each of X⁸, X⁹, X¹⁰, X¹², X¹³, and X¹⁴ is optionally present.

25. A peptide wherein the peptide is or comprises:
B′-X²-X³-J″-X⁵-X⁶-X⁷-X⁸-X⁹-X¹⁰-O′-X¹²-X¹³-X¹⁴,
or a salt thereof, wherein:
B′ is 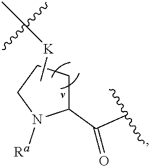
or a salt or a stereoisomeric form thereof, wherein:
v is 1 or 2;
K is a covalent bond, or an substituted or unsubstituted bivalent group selected from a bivalent aliphatic group, alkylene, alkenylene, alkynylene, a bivalent heteroaliphatic group, heteroalkylene, heteroalkenylene, heteroalkynylene, heterocyclene, carbocyclene, arylene, and heteroarylene;
R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group selected from, or one or more combinations of, substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; and substituted or unsubstituted heteroarylene; or R^{a} is or comprises a peptide moiety;
J″ is 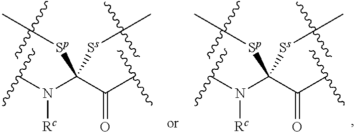
or a salt or a stereoisomeric form thereof, wherein:
S^{p} is —S^{p1}—S^{p2}—S^{p3}—, wherein S^{p1} is bonded to K;
S^{s} is —S^{s1}—S^{s2}—S^{s3}—, wherein S^{s3} is bonded to L₁;
each of S^{p1}, S^{p2} S^{p3}, S^{s1}, S^{s2}, and S^{s3} is independently S^{L};
each S^{L} is independently a bond, a substituted or unsubstituted C₁₋₁₀alkane, a substituted or unsubstituted C₁₋₁₀alkylene, or an optionally substituted, bivalent C₁-C₂₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon;
O′ is of formula: 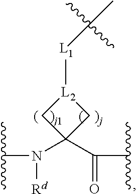
or a salt or stereoisomeric form thereof; wherein:
R^{d} is hydrogen; acyl; substituted or unsubstituted C₁₋₆ alkyl; or an amino protecting group;
L₁ is independently, a bond, a substituted or unsubstituted bivalent C₁₋₁₀ aliphatic or heteroaliphatic, a substituted or unsubstituted C₁₋₁₀ alkylene, —C(O)O—, or —C(═O)OR³—;
L₂ is independently a bond, N, optionally substituted CH, or C(R⁴);
R⁵ is, independently, hydrogen; acyl; substituted or unsubstituted C₁₋₆ alkyl; or an amino protecting group;
each of R³ and R⁴ is independently hydrogen, halogen, —NO₂, —OH, —CN, or C₁₋₆ alkyl;
each of j and j1 is independently 0, 1, 2, 3, 4, 5, 6, 7, 8, 9, or 10; and
each of X², X³, X⁵, X⁶, X⁸, X⁹, X¹⁰, X¹², X¹³, and X¹⁴ is independently an amino acid residue which may be a D stereoisomer or an L stereoisomer; and
each of X⁸, X⁹, X¹⁰, X¹², X¹³, and X¹⁴ is optionally present.

26. A peptide having the structure of: 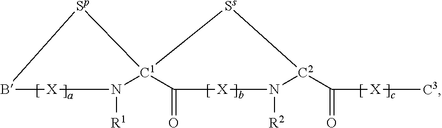
or a salt or a stereoisomer thereof, wherein:
B′ is 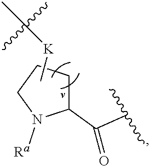
or a salt or stereoisomeric form thereof; wherein:
v is 1 or 2;
K is a hydrogen; a substituted or unsubstituted aliphatic; a substituted or unsubstituted alkylene; a substituted or unsubstituted alkynylene; a substituted or unsubstituted heteroaliphatic; a substituted or unsubstituted aryl; a substituted or unsubstituted heteroaryl; a substituted or unsubstituted acyl; a substituted or unsubstituted hydroxyl; a substituted or unsubstituted thiol; a substituted or unsubstituted amino; or a halo;
R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; or substituted or unsubstituted heteroarylene; or R^{a} is or comprises a peptide moiety;
each of R¹ and R² is independently R′;
C³ is R′, —OR′ or —N(R′)₂;
each X is independently an amino acid residue which may be a D stereoisomer or an L stereoisomer;
each of a, b, and c is independently 1, 2, 3, 4, 5, 6, 7, 8, 9 10, 11, 12, 13, 14, 15, 16, 17, 18, 19, or 20;
C¹ is a carbon atom;
C² is of the formula: 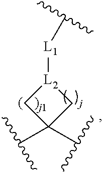
or a salt or stereoisomeric form thereof, wherein:
L₁ is independently, a bond, a substituted or unsubstituted bivalent C₁₋₁₀ aliphatic or heteroaliphatic, a substituted or unsubstituted C₁₋₁₀ alkylene, —C(O)O—, or —C(═O)OR³—;
L₂ is independently a bond, N, optionally substituted CH, or C(R⁴);
R⁵ is, independently, hydrogen; acyl; substituted or unsubstituted C₁₋₆ alkyl; or an amino protecting group;
each of R³ and R⁴ is independently hydrogen, halogen, —NO₂, —OH, —CN, or C₁₋₆ alkyl;
each of j and j1 is independently 0, 1, 2, 3, 4, 5, 6, 7, 8, 9, or 10;
S^{p} is —S^{p1}—S^{p2}—S^{p3}—, wherein S^{p1} is bonded to K and S^{p3} is bonded to C¹;
S^{s} is —S^{s1}—S^{s2}—S^{s3}—, wherein S^{s1} is bonded to C¹ and S^{s3} is bonded to L₁;
each of S^{p1}, S^{p2}, S^{p3}, S^{s1}, S^{s2}, and S^{s3} is independently S^{L};
each S^{L} is independently a bond, a substituted or unsubstituted C₁₋₁₀ alkane, a substituted or unsubstituted C₁₋₁₀ alkylene, or an optionally substituted, bivalent C₁-C₂₀ aliphatic group wherein one or more methylene units of the aliphatic group are optionally and independently replaced with —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)O—, —S(O)—, —S(O)₂—, —S(O)₂N(R′)—, —C(O)S—, or —C(O)O—;
each -Cy- is independently an optionally substituted bivalent group selected from a C₃₋₂₀ cycloaliphatic ring, a C₆₋₂₀ aryl ring, a 5-20 membered heteroaryl ring having 1-10 heteroatoms independently selected from oxygen, nitrogen, sulfur, phosphorus and silicon, and a 3-20 membered heterocyclyl ring having 1-10 heteroatoms independently selected from oxygen, nitrogen, sulfur, phosphorus and silicon;
each R′ is independently —R, —C(O)R, —CO₂R, or —SO₂R;
each R is independently —H, or an optionally substituted group selected from C₁₋₃₀ aliphatic, C₁₋₃₀ heteroaliphatic having 1-10 heteroatoms independently selected from oxygen, nitrogen, sulfur, phosphorus and silicon, C₆₋₃₀ aryl, C₆₋₃₀ arylaliphatic, C₆₋₃₀ arylheteroaliphatic having 1-10 heteroatoms independently selected from oxygen, nitrogen, sulfur, phosphorus and silicon, 5-30 membered heteroaryl having 1-10 heteroatoms independently selected from oxygen, nitrogen, sulfur, phosphorus and silicon, and 3-30 membered heterocyclyl having 1-10 heteroatoms independently selected from oxygen, nitrogen, sulfur, phosphorus and silicon, or
two R groups are optionally and independently taken together to form a covalent bond, or:
two or more R groups on the same atom are optionally and independently taken together with the atom to form an optionally substituted, 3-30 membered, monocyclic, bicyclic or polycyclic ring having, in addition to the atom, 0-10 heteroatoms independently selected from oxygen, nitrogen, sulfur, phosphorus and silicon; or
two or more R groups on two or more atoms are optionally and independently taken together with their intervening atoms to form an optionally substituted, 3-30 membered, monocyclic, bicyclic or polycyclic ring having, in addition to the intervening atoms, 0-10 heteroatoms independently selected from oxygen, nitrogen, sulfur, phosphorus and silicon.

27. The peptide of claim 26, wherein b is 6.

28. The peptide of claim 26, wherein b is 2.

29. The peptide of claim 26, wherein b is 3.

30. The peptide of any one of claims 26-29, wherein a is 2.

31. The peptide of any one of the preceding claims, wherein B is selected from the group consisting of: 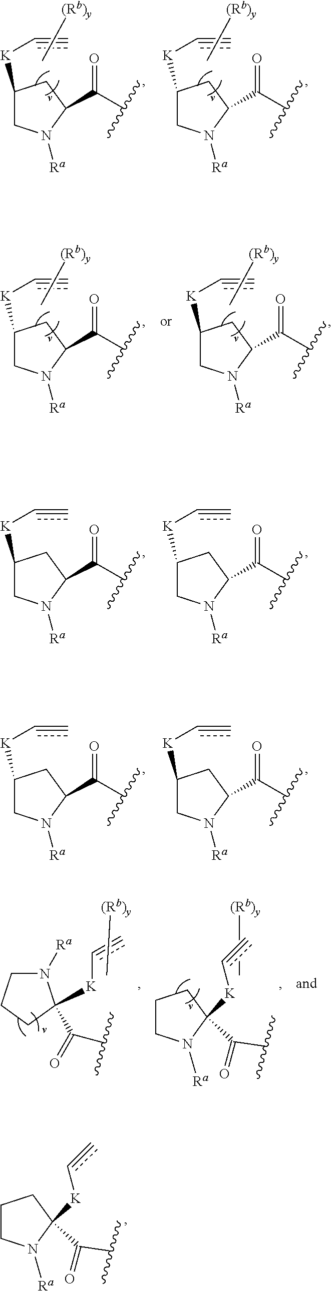
or a salt or stereoisomer thereof; and B′ is selected from 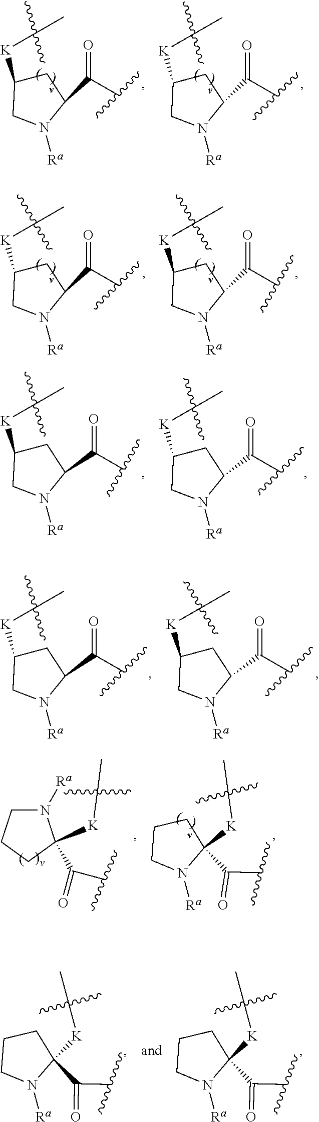
or a salt or a stereoisomeric form thereof.

32. The peptide of any one of the preceding claims, wherein K is optionally substituted C₁₋₁₀alkylene.

33. The peptide of any one of the preceding claims, wherein K is —CH₂—.

34. The peptide of any one of the preceding claims, wherein v is 1.

35. The peptide of any one of the preceding claims, wherein R^{a} is hydrogen, substituted or unsubstituted aliphatic; substituted or unsubstituted heteroaliphatic; substituted or unsubstituted aryl; substituted or unsubstituted heteroaryl; substituted or unsubstituted acyl; a resin; an amino protecting group; or a label optionally joined by a linker, wherein the linker is a group consisting of one or more combinations of substituted or unsubstituted alkylene; substituted or unsubstituted alkenylene; substituted or unsubstituted alkynylene; substituted or unsubstituted heteroalkylene; substituted or unsubstituted heteroalkenylene; substituted or unsubstituted carbocyclene; substituted or unsubstituted heterocyclene; substituted or unsubstituted arylene; or substituted or unsubstituted heteroarylene.

36. The peptide of any one of the preceding claims, wherein R^{a} is substituted or unsubstituted acyl.

37. The peptide of any one of the preceding claims, wherein R^{a} is —C(O)R.

38. The peptide of any one of the preceding claims, wherein R^{a} is —C(O)CH₃.

39. The peptide of any one of the preceding claims, wherein B is 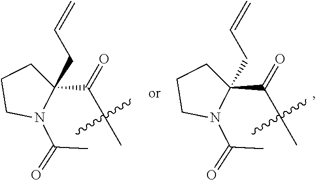 [SMILES: *C(=O)[C@@]1(CC=C)CCCN1C(C)=O ; *C(=O)[C@]1(CC=C)CCCN1C(C)=O]
or B′ is

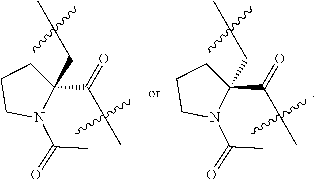 [SMILES: *C[C@]1(C(*)=O)CCCN1C(C)=O ; *C[C@@]1(C(*)=O)CCCN1C(C)=O]

40. The peptide of any one of the preceding claims, wherein B is 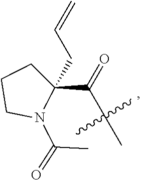 [SMILES: *C(=O)[C@]1(CC=C)CCCN1C(C)=O]
or B′ is

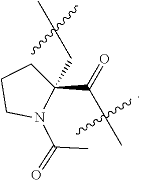 [SMILES: *C[C@@]1(C(*)=O)CCCN1C(C)=O]

41. The peptide of any one of the preceding claims, wherein S^{p} is —CH═CH—(CH₂)n-, wherein n is 1-10.

42. The peptide of any one of the preceding claims, wherein S^{s} is —(CH₂)mCH═CH—(CH₂)n-, wherein each of m and n is independently 1-10.

43. The peptide of any one of claims 41-42, wherein n is 3.

44. The peptide of any one of the preceding claims, wherein J is 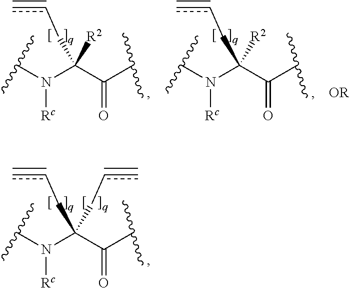
or a salt or stereoisomeric form thereof, wherein:
each instance of q is independently 1, 2, or 3; and
each instance of [symbol] independently represents a single bond, a double bond or a triple bond.

45. The peptide of any one of the preceding claims, wherein X⁶ is Z.

46. The peptide of any one of the preceding claims, wherein X⁵ is Z.

47. The peptide of any one of the preceding claims, wherein Z is a leucine residue.

48. The peptide of any one of the preceding claims, wherein the peptide binds to an estrogen receptor with a half maximal effective concentration (EC50) of less than about 3.0 uM.

49. The peptide of any one of the preceding claims, wherein the peptide binds to the estrogen receptor with an EC50 of less than about 1.0 uM.

50. The peptide of any one of the receding claims, wherein J is of the formula: 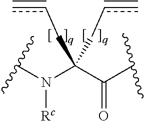
or a salt or stereoisomeric thereof, and
X⁸ is of the formula: 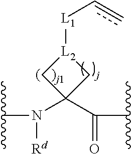
or a salt or stereoisomeric form thereof, wherein:
R^{d} is hydrogen; acyl; substituted or unsubstituted C₁₋₆ alkyl; or an amino protecting group;
L₁ is independently, a bond, a substituted or unsubstituted bivalent C₁₋₁₀ aliphatic or heteroaliphatic, a substituted or unsubstituted C₁₋₁₀ alkylene, —C(O)O—, or —C(═O)OR³—;
L₂ is independently a bond, N, optionally substituted CH, or C(R⁴);
R⁵ is, independently, hydrogen; acyl; substituted or unsubstituted C₁₋₆ alkyl; or an amino protecting group;
each of R³ and R⁴ is independently hydrogen, halogen, —NO₂, —OH, —CN, or C₁₋₆ alkyl;
each of j and j1 is independently 0, 1, 2, 3, 4, 5, 6, 7, 8, 9, or 10; and
each instance of [symbol] independently represents a single bond, a double bond or a triple bond.

51. The peptide of any one of the preceding claims, wherein:
J″ is 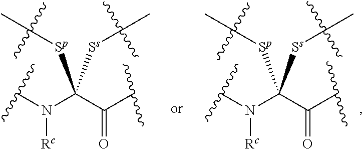
or a salt or a stereoisomeric form thereof, and
X⁸ is of the formula: 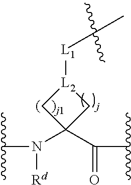
or a salt or stereoisomeric form thereof; wherein:
R^{d} is hydrogen; acyl; substituted or unsubstituted C₁₋₆ alkyl; or an amino protecting group;
L₁ is independently, a bond, a substituted or unsubstituted bivalent C₁₋₁₀ aliphatic or heteroaliphatic, a substituted or unsubstituted C₁₋₁₀ alkylene, —C(O)O—, or —C(═O)OR³—;
L₂ is independently a bond, N, optionally substituted CH, or C(R⁴);
R⁵ is, independently, hydrogen; acyl; substituted or unsubstituted C₁₋₆ alkyl; or an amino protecting group;
each of R³ and R⁴ is independently hydrogen, halogen, —NO₂, —OH, —CN, or C₁₋₆ alkyl;
each of j and j1 is independently 0, 1, 2, 3, 4, 5, 6, 7, 8, 9, or 10.

52. The peptide of any one of the preceding claims, wherein J is of the formula: 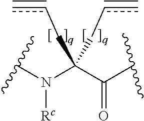
or a salt or stereoisomeric form thereof;
each of X⁹, X¹⁰, and X¹ is present; and
X¹¹ is: 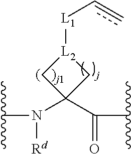
or a salt or stereoisomeric form thereof, wherein:
R^{d} is hydrogen; acyl; substituted or unsubstituted C₁₋₆ alkyl; or an amino protecting group;
L₁ is independently, a bond, a substituted or unsubstituted bivalent C₁₋₁₀ aliphatic or heteroaliphatic, a substituted or unsubstituted C₁₋₁₀ alkylene, —C(O)O—, or —C(═O)OR³—;
L₂ is independently a bond, N, optionally substituted CH, or C(R⁴);
R⁵ is, independently, hydrogen; acyl; substituted or unsubstituted C₁₋₆ alkyl; or an amino protecting group;
each of R³ and R⁴ is independently hydrogen, halogen, —NO₂, —OH, —CN, or C₁₋₆ alkyl;
each of j and j1 is independently 0, 1, 2, 3, 4, 5, 6, 7, 8, 9, or 10; and
each instance of [symbol] independently represents a single bond, a double bond or a triple bond.

53. The peptide of any one of the preceding claims, wherein:
J″ is 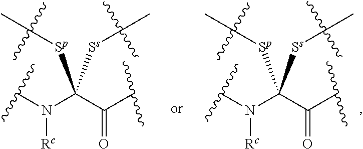
or a salt or a stereoisomeric form thereof, and
each of X⁹, X¹⁰, and X¹¹ is present; and
X¹¹ is: 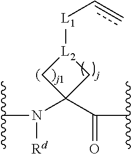
or a salt or stereoisomeric form thereof, wherein: 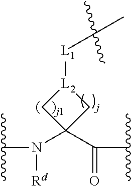
or a salt or stereoisomeric form thereof; wherein:
R^{d} is hydrogen; acyl; substituted or unsubstituted C₁₋₆ alkyl; or an amino protecting group;
L₁ is independently, a bond, a substituted or unsubstituted bivalent C₁₋₁₀ aliphatic or heteroaliphatic, a substituted or unsubstituted C₁₋₁₀ alkylene, —C(O)O—, or —C(═O)OR³—;
L₂ is independently a bond, N, optionally substituted CH, or C(R⁴);
R⁵ is, independently, hydrogen; acyl; substituted or unsubstituted C₁₋₆ alkyl; or an amino protecting group;
each of R³ and R⁴ is independently hydrogen, halogen, —NO₂, —OH, —CN, or C₁₋₆ alkyl;
each of j and j1 is independently 0, 1, 2, 3, 4, 5, 6, 7, 8, 9, or 10.

54. The peptide of any one of the preceding claims, wherein the peptide can form a helix structure.

55. The peptide of any one of the preceding claims, wherein the peptide comprises no more than 1 or 2 unmasked amide NH.

56. The peptide of any one of the preceding claims, wherein the peptide comprises no more than 1 unmasked amide NH.

57. A pharmaceutical composition which comprises or delivers a peptide of any of the preceding claims, or a pharmaceutically acceptable salt thereof, and a pharmaceutically acceptable carrier and/or a pharmaceutically acceptable excipient.

58. A method of treating a disease in a subject comprising administering to the subject a therapeutically effective amount of a peptide or composition of any one of the preceding claims.

59. The method of claim 58, wherein the subject is a human.

60. The method of claim 58 or 59, wherein the disease is cancer.

61. The peptide of any one of the preceding claims, wherein X² is an amino acid residue or a homolog thereof selected from a leucine amino acid residue, an isoleucine amino acid residue, an alanine amino acid residue, a cyclopropyl alanine amino acid residue, a lysine amino acid residue, and a threonine amino acid residue, wherein the homolog may be a D stereoisomer or an L stereoisomer.

62. The peptide of claim 51, wherein X² is a leucine amino acid residue.

63. The peptide of claim 51, wherein X² is an alanine amino acid residue.

64. The peptide of any one of the preceding claims, wherein X³ is an amino acid residue or a homolog thereof selected from a histidine amino acid residue, a norleucine amino acid residue, a leucine amino acid residue, and an arginine amino acid residue, wherein the homolog may be a D stereoisomer or an L stereoisomer.

65. The peptide of claim 54, wherein X³ is a leucine amino acid residue.

66. The peptide of any one of the preceding claims, wherein X⁵ is an amino acid residue or a homolog thereof selected from an arginine amino acid residue, an asparagine amino acid residue, a leucine amino acid residue, a tyrosine amino acid residue, a norleucine amino acid residue, a cyclopropyl alanine amino acid residue, and a histidine amino acid residue, wherein the homolog may be a D stereoisomer or an L stereoisomer.

67. The peptide of claim 56, wherein X⁵ is an arginine amino acid residue.

68. The peptide of claim 56, wherein X⁵ is a tyrosine amino acid residue.

69. The peptide of any one of the preceding claims, wherein X⁶ is an amino acid residue or a homolog thereof selected from a leucine amino acid residue, a histidine amino acid residue, a tyrosine amino acid residue, and a norleucine amino acid residue, wherein the homolog may be a D stereoisomer or an L stereoisomer.

70. The peptide of claim 59, wherein X⁶ is a leucine amino acid residue.

71. The peptide of any one of the preceding claims, wherein X⁷ is an amino acid residue or a homolog thereof selected from a leucine amino acid residue, a glutamine amino acid residue, a histidine amino acid residue, and an alanine amino acid residue, wherein the homolog may be a D stereoisomer or an L stereoisomer.

72. The peptide of claim 61, wherein X⁷ is a leucine amino acid.

73. The peptide of any one of the preceding claims, wherein X⁸ is an amino acid residue or a homolog thereof selected from an glutamine amino acid residue, a leucine amino acid residue, a histidine amino acid residue, a threonine amino acid residue, an alanine amino acid residue, a tyrosine amino acid residue, an aspartic acid amino acid residue, and an asparagine amino acid residue, wherein the homolog may be a D stereoisomer or an L stereoisomer.

74. The peptide of claim 63, wherein X⁸ is a glutamine amino acid residue.

75. The peptide of claim 63, wherein X⁸ is an aspartic acid amino acid residue.

76. The peptide of claim 63, wherein X⁸ is a tyrosine amino acid residue.

77. The peptide of any one of the preceding claims, wherein X⁹ is an amino acid residue or a homolog thereof selected from a tyrosine amino acid residue, an aspartic acid amino acid residue, and an asparagine amino acid residue, wherein the homolog may be a D stereoisomer or an L stereoisomer.

78. The peptide of claim 67, wherein X⁹ is an aspartic acid amino acid residue.

79. The peptide of claim 67, wherein In some embodiments, X⁹ is a tyrosine amino acid residue.
